SEQUENCE LISTING (44 sequences)

SEQ ID NO: 1 - DNA, 20 bp, Homo sapiens
cgttcatgtgccttgtgtgt

SEQ ID NO: 2 - DNA, 20 bp, Homo sapiens
tcattgcagtgcttcctgtt

SEQ ID NO: 3 - DNA, 21 bp, Homo sapiens
tctcttgacttctggcagagc

SEQ ID NO: 4 - DNA, 20 bp, Homo sapiens
gtgtgagccgaaagaaaagg

SEQ ID NO: 5 - DNA, 20 bp, Homo sapiens
ttaaagcgcctctgtgattg

SEQ ID NO: 6 - DNA, 18 bp, Homo sapiens
cgcgaacggctgcttatt

SEQ ID NO: 7 - DNA, 20 bp, Homo sapiens
tttgtgaaggaggacactgg

SEQ ID NO: 8 - DNA, 19 bp, Homo sapiens
ctgagcgctgagaagcaag

SEQ ID NO: 9 - DNA, 20 bp, Homo sapiens
aaatcatccgaccgttctca

SEQ ID NO: 10 - DNA, 20 bp, Homo sapiens
aggatctcccggttaggaaa

SEQ ID NO: 11 - DNA, 18 bp, Homo sapiens
gtcgtccccctcctccat

SEQ ID NO: 12 - DNA, 18 bp, Homo sapiens
actgcagcctggccttgt

SEQ ID NO: 13 - DNA, 20 bp, Homo sapiens
gggaaagcaagcagagacca

SEQ ID NO: 14 - DNA, 20 bp, Homo sapiens
ttgcctccgaaccagctttc

SEQ ID NO: 15 - DNA, 21 bp, Homo sapiens
ttcagagcccagtgtaagagc

SEQ ID NO: 16 - DNA, 27 bp, Homo sapiens
tgtctatcaatgtcttgagaagtctac

SEQ ID NO: 17 - DNA, 20 bp, Homo sapiens
cattttgcatctgggctaca

SEQ ID NO: 18 - DNA, 20 bp, Homo sapiens
ggatgccagtcaaggaaaat

SEQ ID NO: 19 - DNA, 20 bp, Homo sapiens
cgaaccccagcaagaaatag

SEQ ID NO: 20 - DNA, 20 bp, Homo sapiens
acaaagcgaccacagctttt

SEQ ID NO: 21 - DNA, 20 bp, Homo sapiens
ccccctgctctctctgtatg

SEQ ID NO: 22 - DNA, 20 bp, Homo sapiens
atgaacacatttgccacagc

SEQ ID NO: 23 - DNA, 20 bp, Homo sapiens
accttttcaccccagctttc

SEQ ID NO: 24 - DNA, 20 bp, Homo sapiens
attccccttcctgtgttgag

SEQ ID NO: 25 - DNA, 21 bp, Homo sapiens
ctaaagcgcatgctccagact

SEQ ID NO: 26 - DNA, 20 bp, Homo sapiens
tgccctttccttaaaagtgg

SEQ ID NO: 27 - DNA, 20 bp, Homo sapiens
gcctactccgacacccaata

SEQ ID NO: 28 - DNA, 23 bp, Homo sapiens
aaggtagagtgatgaaagttgtt

SEQ ID NO: 29 - DNA, 21 bp, Homo sapiens
caccatgtcctctgtctattc

SEQ ID NO: 30 - DNA, 24 bp, Homo sapiens
tacactattgaatcacagggactt

SEQ ID NO: 31 - DNA, 24 bp, Homo sapiens
aggagatgagagccaaggtcattg

SEQ ID NO: 32 - DNA, 24 bp, Homo sapiens
ttttcctcctgtggtggattcttg

SEQ ID NO: 33 - DNA, 20 bp, Homo sapiens
tgcctcttgacatttggaca

SEQ ID NO: 34 - DNA, 20 bp, Homo sapiens
gtttctgccggtaccagtgt

SEQ ID NO: 35 - DNA, 56 bp, Homo sapiens
cagcagcagacccgagacgtagtattcccaccacacgccaccttcataaacgtgcg

SEQ ID NO: 36 - DNA, 56 bp, Homo sapiens
cagcagcagacccgagacgtagtaggaaacaaacacgccaccttcataaacgtgcg

SEQ ID NO: 37 - DNA, 3047 bp, Homo sapiens
cccgccagctcgcggagccgctctgccccgcgccctagcccgcgcctgcagcccgcccaggcggagtcagcccgcgctccgcccgccgcgatccgagctcggaggttcggactccgggctcgccgccccccgggccggctccgcgccccgcactcccggcgcccagcgccccgcgccccggcgggcggagcgcaccatgccgcagctggactccggcgggggcggcgcgggcggcggcgacgacctcggcgcgccggacgagctgctggccttccaggatgaaggcgaggagcaggacgacaagagccgcgacagcgccgccggtcccgagcgcgacctggccgagctcaagtcgtcgctcgtgaacgagtccgagggcgcggccggcggcgcagggatcccgggggtcccgggggccggcgccggggcccgcggcgaggccgaggctctcgggcgggaacacgctgcgcagagactcttcccggacaaacttccagagcccctggaggacggcctgaaggccccggagtgcaccagcggcatgtacaaagagaccgtctactccgccttcaatctgctcatgcattacccacccccctcgggagcagggcagcacccccagccgcagcccccgctgcacaaggccaatcagcccccccacggtgtcccccaactctctctctacgaacatttcaacagcccacatcccacccctgcacctgcggacatcagccagaagcaagttcacaggcctctgcagacccctgacctctctggcttctactccctgacctcaggcagcatggggcagctcccccacactgtgagctggttcacccacccatccttgatgctaggttctggtgtacctggtcacccagcagccatcccccacccggccattgtgcccccctcagggaagcaggagctgcagcccttcgaccgcaacctgaagacacaagcagagtccaaggcagagaaggaggccaagaagccaaccatcaagaagcccctcaatgccttcatgctgtacatgaaggagatgagagccaaggtcattgcagagtgcacacttaaggagagcgctgccatcaaccagatcctgggccgcaggtggcacgcgctgtcgcgagaagagcaggccaagtactatgagctggcccgcaaggagaggcagctgcacatgcagctatacccaggctggtcagcgcgggacaactacgggaagaagaagaggcggtcgagggaaaagcaccaagaatccaccacaggaggaaaaagaaatgcattcggtacttacccggagaaggccgctgccccagccccgttccttccgatgacagtgctctaggctgccccgggtccccagctccccaggactcaccctcataccatctgctgccccgcttccccacagaactgcttactagccctgcggagccggcacctacatccccaggtctctccactgctctcagcctcccaaccccagggcccccacaggccccccgcagcaccctgcagagcacacaggtacagcaacaggaatctcagagacaggtggcctagcaggcacaggacacctggccgcctccaggagcctaccccctgaaagtgacagagacccagatctcatggaaactggccaggggtcctgttaacgtcatctcagggtccagaccctgaagatttcagaggctgcagaacttctgcctgaacctggggtcatcgattcaaactgctccaagtggtgggaatcagatctgtcttgatgtgtcatctaattaagggaatcccttgtacctatggctgcctgcatctattctttgtaccatctgtcttgccagccagaagcctctgcctccctagcttttctgctataggtcagagatgggctgaactgagcctagctaccttctctacccatctcccccatcccccactgccacaccctccccattcagacacttcatggaccaagaatgagctggtttgtcaaacaacatgtgagcatggtcacaagcacaaagctcaagatgacagctcttctaaggaaatggagaagctctgtttataaaaacaaaaacaaaaccagctgctactcataagttggaccagaggaagccccttactatgatctcaggagcttgcaagaagcaggaaggggaatggaataggttaagtttaggcctatcaacctaagcaacagaaataatctgacactaccttatcaggcaaattggggaggggagggtgtatctagctctagttcaaattatttgaaagtgttccctgagaaacccaccagcctaagaagctctggccccaggcttgtcactagcagctgcagtcaacagttcaaagaagtcatggcccaaatccagtgtgcacccctccccattcacagagcctttttcacaattccatttccagttcatctatggcagtccagccagctcctgggcagcttgagagggcaaacccaaaacctcatgacagccagagcctgtctttcagcattcagtccgcctggccggctccagtttccccatggggctgcgggacagaggaccattacaactagatcaaggagcccagaaaacctccagtagtggacaacaggttttcaccatagcctacgttaacccatttttgagccaagcttcaaccctcagccttgaaaaacaagtctttaatttaatttttgttttttgcctaaatccaaagaaaaagggctgtcgggccaggcgcggtggctcacgcctgtaatcccagcactttggcaggccgaggcaggtggatcacctgacgtcagtagtttgagaccagcctggccaacatggtgaaaccctgtatctactaaaaatacaaaaattagccggacgtggtggtgcgcgcatgtaatcccagctactcgggaggctgaggcggaagaatcccttgaacccgggaggcggaggttccagtgagccgaggtggcgctattgcactccagtctgggtaacagggagactgcatctcaaaaaaaaaaaaaaaaaaaaaaaaaa

SEQ ID NO: 38 - protein, 384 aa, Homo sapiens
MPQLDSGGGGAGGGDDLGAPDELLAFQDEGEEQDDKSRDSAAGPERDLAELKSSLVNESEGAAGGAGIPGVPGAGAGARGEAEALGREHAAQRLFPDKLPEPLEDGLKAPECTSGMYKETVYSAFNLLMHYPPPSGAGQHPQPQPPLHKANQPPHGVPQLSLYEHFNSPHPTPAPADISQKQVHRPLQTPDLSGFYSLTSGSMGQLPHTVSWFTHPSLMLGSGVPGHPAAIPHPAIVPPSGKQELQPFDRNLKTQAESKAEKEAKKPTIKKPLNAFMLYMKEMRAKVIAECTLKESAAINQILGRRWHALSREEQAKYYELARKERQLHMQLYPGWSARDNYGKKKRRSREKHQESTTGGKRNAFGTYPEKAAAPAPFLPMTVL

SEQ ID NO: 39 - DNA, 4078 bp, Homo sapiens
ggtttccaggcctgaggtgcccgccctggccccaggagaatgaaccagccgcagaggatggcgcctgtgggcacagacaaggagctcagtgacctcctggacttcagcatgatgttcccgctgcctgtcaccaacgggaagggccggcccgcctccctggccggggcgcagttcggaggttcaggtcttgaggaccggcccagctcaggctcctggggcagcggcgaccagagcagctcctcctttgaccccagccggaccttcagcgagggcacccacttcactgagtcgcacagcagcctctcttcatccacattcctgggaccgggactcggaggcaagagcggtgagcggggcgcctatgcctccttcgggagagacgcaggcgtgggcggcctgactcaggctggcttcctgtcaggcgagctggccctcaacagccccgggcccctgtccccttcgggcatgaaggggacctcccagtactacccctcctactccggcagctcccggcggagagcggcagacggcagcctagacacgcagcccaagaaggtccggaaggtcccgccgggtcttccatcctcggtgtacccacccagctcaggtgaggactacggcagggatgccaccgcctacccgtccgccaagacccccagcagcacctatcccgcccccttctacgtggcagatggcagcctgcacccctcagccgagctctggagtcccccgggccaggcgggcttcgggcccatgctgggtgggggctcatccccgctgcccctcccgcccggtagcggcccggtgggcagcagtggaagcagcagcacgtttggtggcctgcaccagcacgagcgtatgggctaccagctgcatggagcagaggtgaacggtgggctcccatctgcatcctccttctcctcagcccccggagccacgtacggcggcgtctccagccacacgccgcctgtcagcggggccgacagcctcctgggctcccgagggaccacagctggcagctccggggatgccctcggcaaagcactggcctcgatctactccccggatcactcaagcaataacttctcgtccagcccttctacccccgtgggctccccccagggcctggcaggaacgtcacagtggcctcgagcaggagcccccggtgccttatcgcccagctacgacgggggtctccacggcctgcagagtaagatagaagaccacctggacgaggccatccacgtgctccgcagccacgccgtgggcacagccggcgacatgcacacgctgctgcctggccacggggcgctggcctcaggtttcaccggccccatgtcactgggcgggcggcacgcaggcctggttggaggcagccaccccgaggacggcctcgcaggcagcaccagcctcatgcacaaccacgcggccctccccagccagccaggcaccctccctgacctgtctcggcctcccgactcctacagtgggctagggcgagcaggtgccacggcggccgccagcgagatcaagcgggaggagaaggaggacgaggagaacacgtcagcggctgaccactcggaggaggagaagaaggagctgaaggccccccgggcccggaccagcagtacggacgaggtgctgtccctggaggagaaagacctgagggaccgggagaggcgcatggccaataacgcgcgggagcgggtgcgcgtgcgggatattaacgaggccttccgggagctggggcgcatgtgccagatgcacctcaagtcggacaaagcgcagaccaagctgctcatcctgcagcaggccgtgcaggtcatcctggggctggagcagcaggtgcgagagcggaacctgaatcccaaagcagcctgtttgaaacggcgagaagaggaaaaggtgtcaggtgtggttggagacccccagatggtgctttcagctccccacccaggcctgagcgaagcccacaaccccgccgggcacatgtgaaagtaaacaaaacctgaaagcaagcaacaaaacatacactttgtcagagaagaaaaaaatgccttaactataaaaagcggagaaatggaaacatatcactcaagggggatgctgtggaaacctggcttattcttctaaagccaccagcaaattgtgcctaagcgaaatattttttttaaggaaaataaaaacattagttacaagattttttttttcttaatgtagatgaaaattagcaaggatgctgcctttggtctctggtttttttaagctttttttgcatatgttttgtaagcaacaaatttttttgtataaaagtcccgtgtctctcgctatttctgctgctgttcctagactgagcattgcatttcttgatcaaccagatgattaaacgttgtattaaaaagaccccgtgtaaacctgagcccccccgtccccccccccccccggaagccactgcacacagacagaacggggacaggcggcgggtcttttgtttttttgatgttgggggttctcttggttttgtcatgtggaaagtgatgcgtgggcgttccctgatgaaggcaccttggggcttccctgccgcatcctctcccctcaggaaggggactgacctgggcttgggggaagggacgtcagcaaggtggctctgaccctcccaggtgactctgccaagcagctgtggcccccagggctaccctacacaacgccctccccaggcccccctaagctgctctcccttggaacctgcacagctctctgaaatggggcattttgttgggaccagtgacccctggcatggggaccacaccctggagcccggtgctggggacctcctggacaccctgtccttcactcctttgccccagggacccaggctcatgctctgaactctggctgagaggatgctgctcaggagccagcacaggacaccccccaccccaccccaccatgtccccattacaccagagggccatcgtgacgtagacaggatgccaggggcctggccagcctcccccaatgctggggagcatccctgggcctggggccacacctgctgccctccctctgtgtggtccaagggcaagagtggctggagccgggggactgtgctggtctgagccccacgaaggccttgggctgtgcgtccgaccctgctgcagaaccagcagggtgtcccctcgggcccatctgtgtcccatgtcccagcacccaggcctctctccaggtctccttttctggtcttttgccatgagggtaaccagctcttcccagctggctggggactgtcttgggtttaaaactgcaagtctcctaccctgggatcccatccagttccacacgaactagggcagtggtcactgtggcacccaggtgtgggcctggctagctgggggccttcatgtgcccttcatgcccctccctgcattgaggccttgtggacccctgggctggctgtgttcatccccgctgcaggtcgggcgtctccccccgtgccactcctgagactcccaccgttacccccaggagatcctggactgcctgactcccctccccagactggcttgggagcctgggccccatggtagatgcaagggaaacctcaaggccagctcaatgcctggtatctgcccccagtccaggccaggcggaggggaggggctgtccggctgcctctcccttctcggtggcttcccctacgccctgggagtttgatctcttaagggaacttgcctctccctcttgttttgctcctggccctgcccctaggtctgggtgggcagtggccccatagcctctggaactgtgcgttctgcatagaattcaaacgagattcacccagcgcgaggaggaagaaacagcagttcctgggaaccacaattatggggggtggggggtgtgatctgagtgcctcaagatggttttcaaaaaaatttttttaaagaaaataattgtatacgtgtcaacacagctggctggatgattgggactttaaaacgaccctctttcaggtggattcagagacctgtcctgtatataacagcactgtagcaataaacgtgacattttataacgatgc

SEQ ID NO: 40 - protein, 651 aa, Homo sapiens
MNQPQRMAPVGTDKELSDLLDFSMMFPLPVTNGKGRPASLAGAQFGGSGLEDRPSSGSWGSGDQSSSSFDPSRTFSEGTHFTESHSSLSSSTFLGPGLGGKSGERGAYASFGRDAGVGGLTQAGFLSGELALNSPGPLSPSGMKGTSQYYPSYSGSSRRRAADGSLDTQPKKVRKVPPGLPSSVYPPSSGEDYGRDATAYPSAKTPSSTYPAPFYVADGSLHPSAELWSPPGQAGFGPMLGGGSSPLPLPPGSGPVGSSGSSSTFGGLHQHERMGYQLHGAEVNGGLPSASSFSSAPGATYGGVSSHTPPVSGADSLLGSRGTTAGSSGDALGKALASIYSPDHSSNNFSSSPSTPVGSPQGLAGTSQWPRAGAPGALSPSYDGGLHGLQSKIEDHLDEAIHVLRSHAVGTAGDMHTLLPGHGALASGFTGPMSLGGRHAGLVGGSHPEDGLAGSTSLMHNHAALPSQPGTLPDLSRPPDSYSGLGRAGATAAASEIKREEKEDEENTSAADHSEEEKKELKAPRARTSSTDEVLSLEEKDLRDRERRMANNARERVRVRDINEAFRELGRMCQMHLKSDKAQTKLLILQQAVQVILGLEQQVRERNLNPKAACLKRREEEKVSGVVGDPQMVLSAPHPGLSEAHNPAGHM

SEQ ID NO: 41 - DNA, 8332 bp, Homo sapiens
gtgtgtggatgtgtgagtgagagggaacgagagtaagagaaagaaagaagtgaggggatgtaaactcgaataaatttcaaagtgcctccgagggatgcaacgggcaaaaactgaactgttcaggcttcagattgtaactgacgatctgaggaaaaatgaggtgctcgatgaattttcgtttgtattttttggcgaggcgggggaggtgttgagattttttttttttcccctcggggtgggtgcgagggggatgcatcctagcctgcccgacccggagcaagtcgcgtctccccgccggagcccccccacccatttctttgctgaacttgcaattccgtgcgcctcggcgtgtttccccctccccccttccctccgtcccctcccctccccggagaagagagttggtgttaagagtcagggatcttggctgtgtgtctgcggatctgtagtggcggcggcggcggcggcggcggggaggcagcaggcgcgggagcgggcgcaggagcaggcggcggcggtggcggcggcggttagacatgaacgccgcctcggcgccggcggtgcacggagagccccttctcgcgcgcgggcggtttgtgtgattttgctaaaatgcatcaccaacagcgaatggctgccttagggacggacaaagagctgagtgatttactggatttcagtgcgatgttttcacctcctgtgagcagtgggaaaaatggaccaacttctttggcaagtggacattttactggctcaaatgtagaagacagaagtagctcagggtcctgggggaatggaggacatccaagcccgtccaggaactatggagatgggactccctatgaccacatgaccagcagggaccttgggtcacatgacaatctctctccaccttttgtcaattccagaatacaaagtaaaacagaaaggggctcatactcatcttatgggagagaatcaaacttacagggttgccaccagcagagtctccttggaggtgacatggatatgggcaacccaggaaccctttcgcccaccaaacctggttcccagtactatcagtattctagcaataatccccgaaggaggcctcttcacagtagtgccatggaggtacagacaaagaaagttcgaaaagttcctccaggtttgccatcttcagtctatgctccatcagcaagcactgccgactacaatagggactcgccaggctatccttcctccaaaccagcaaccagcactttccctagctccttcttcatgcaagatggccatcacagcagtgacccttggagctcctccagtgggatgaatcagcctggctatgcaggaatgttgggcaactcttctcatattccacagtccagcagctactgtagcctgcatccacatgaacgtttgagctatccatcacactcctcagcagacatcaattccagtcttcctccgatgtccactttccatcgtagtggtacaaaccattacagcacctcttcctgtacgcctcctgccaacgggacagacagtataatggcaaatagaggaagcggggcagccggcagctcccagactggagatgctctggggaaagcacttgcttcgatctattctccagatcacactaacaacagcttttcatcaaacccttcaactcctgttggctctcctccatctctctcagcaggcacagctgtttggtctagaaatggaggacaggcctcatcgtctcctaattatgaaggacccttacactctttgcaaagccgaattgaagatcgtttagaaagactggatgatgctattcatgttctccggaaccatgcagtgggcccatccacagctatgcctggtggtcatggggacatgcatggaatcattggaccttctcataatggagccatgggtggtctgggctcagggtatggaaccggccttctttcagccaacagacattcactcatggtggggacccatcgtgaagatggcgtggccctgagaggcagccattctcttctgccaaaccaggttccggttccacagcttcctgtccagtctgcgacttcccctgacctgaacccaccccaggacccttacagaggcatgccaccaggactacaggggcagagtgtctcctctggcagctctgagatcaaatccgatgacgagggtgatgagaacctgcaagacacgaaatcttcggaggacaagaaattagatgacgacaagaaggatatcaaatcaattactaggtcaagatctagcaataatgacgatgaggacctgacaccagagcagaaggcagagcgtgagaaggagcggaggatggccaacaatgcccgagagcgtctgcgggtccgtgacatcaacgaggctttcaaagagctcggccgcatggtgcagctccacctcaagagtgacaagccccagaccaagctcctgatcctccaccaggcggtggccgtcatcctcagtctggagcagcaagtccgagaaaggaatctgaatccgaaagctgcgtgtctgaaaagaagggaggaagagaaggtgtcctcagagcctccccctctctccttggccggcccacaccctggaatgggagacgcatcgaatcacatgggacagatgtaaaagggtccaagttgccacattgcttcattaaaacaagagaccacttccttaacagctgtattatcttaaacccacataaacacttctccttaacccccatttttgtaatataagacaagtctgagtagttatgaatcgcagacgcaagaggtttcagcattcccaattatcaaaaaacagaaaaacaaaaaaaagaaagaaaaaagtgcaacttgagggacgactttctttaacatatcattcagaatgtgcaaagcagtatgtacaggctgagacacagcccagagactgaacggcaatctttccacactgtggaacaatgcatttgtgcctaaacttcttttggaaaaaaaaaatataattaatttgtaagtctgaaaaaaaaatatttaatttaaaaaaaattgtaaacttgcaataatgaaaaagtgtacttctgaagaaaactacatgaacgtttttgttggtattcaagtcagctagtgtttataattactggatattgaattaggggaagctcggctgccctagtaacaaaaccagcaaacgtcctgatgacaacgaagtgatgacattagccattccttagggtaggaggaacagatggatcttatagacctatgacaaatatatatataaatatatatataaatatatattaaaaatttagtgactatggtaagcttttgttcatttgtttcagacttttttctcctgtaaaaaaatagtactgattaacttttttaaaagaaagattttactgtaaatatggatttttttttttttggtcttatttctgtccctttccctggtttgttatcgtaacctgtagtgccaactctgcttccagaggggtagtgcaggatgaaatgctgaccctgatgttgcttctcattcataaataagtagaaagttgtttctccagtcttttgggaacacaggacttaaaagtcacatcatgtgtagatattacaagcagcattaccaagacatggcaaaaagagtttgtctgaattgtaatgttgcgtttgtgaacctattctgggattttcagaggtacaaggttagaatgctacaatgttaccactgtgccttccaatgtttatatcatcggaaacataacataatcaaagtggctgtgatttaacaaaatgattaaagtgttacctacctgtgtagccgaagtagtgtgcagtgaggcgtttctgaatacatggtcagatttttggaaaaaaacaaaaacaaaaaaaacaagtaaagttcaaaaaccgtcaaatgagaaaattgcaagtagtgtgacagagctgattgattttgttgctttcttgattttttttttcaaaatgggtttactaaaatgtagatgacttaactgcctcctccttcgtctgaaaaatgccaatattcaatcatcatgcagcattataacaagccttataagtcctaaagcattaagttgcacttttttgaggaggggtagtgcagtatttctctggccagtatgaatgaagtttatacttaccatatttgatagaaacatagatcaagctatggcacagcgactcatcagatagctagctttgacgtctgggcacaattgaaccaacttccatcgtgaatctttataatgattgactttggtgtatagtgcagtaaacaaatagtgctcctagttaagtatttgtcagcatccttttgtctctaacttgtttctatttttacagccacacaattcttggcatgtattaagaaaaaaaaaaatccctgttcaagtagtttttccacctatcagcactgagtaaatgccataaatccattgaaatggtctaaatgttccatctgttctcctgttttgccagttatatagtaatgaaatacatttgtaaattttatgcaacaaatggcaaacgtatcattattttgaaattgtgtatgtaaaagttatatttttacatgtagactcttgttattatgtgttttaatacattgtatcagtttttgtttttttttaaaaactgtggtttaaaaagaagtctcatttaaatgaaatagctacaagaatcagaattttatgttcatttctgaaaatgtaagaacaaataagatagttaccacgtggtcatcttttacaaacccataaacattttgattagctgtgtgtgtgttgaaaaactgtaaatatgttcagtagcgataaaactaaaataactttgatttgttgataagttcctaaaatgtggaggtggattaaaaccttaggagaatagcagaaatcaaacttcatgaaaagttattttggggctttcctgtgaaatgtatgaacaaagaggctcagagaaggacatggaagacaataatgtatactctctcctcctccctgaataatgaaaaccatgtgtatttgttccctccgtatgttaaagatttccttttagtggtacattctgcactcattttgtatagtctaccaaggcgggtatccctaggaacaatattatataggaagcaggtatactctgatcacattcaggataagtgtacagaagaaaatacggtgtttactctttagggaactggaaacactccctgcattgatgtacattttaagaatggcacttttgatacatgttatcataaaggtgcttaatagagctgaattaaagtttttcaaatctgtaaacaaagcaaaaaagtaaattgtagtcatttgattattttttaaattggtgctttatattttgttctcactcagagtaaaagctgcaatttattgttcaccagctttgatgtattcattactcagtaatgcaatacctctattgttgaattccctttggaaataagtgaaaattctaacggccactgaaagctgctcgctaggttttgcttggtggagaaacataatctgcacctatccatattaattgggttgtatccccattaaaaaagaaaaaaagggaatgtggcctttttagtgtgttttttattgttgttgttttgtaattatcaaacccaggtaagatattggtatcctgcactggattttcaaatgaagttcagcagaagacagttaagattaaagtactatacaaaaatttcaaaagggtccatactacgctatctgtatgacgacacttaggctggggatctctttcagaaactcggactttaaaagcaacttggagcagttgatccacctccacattcaagtaatttatgaatatgcagaatagggatctgttcatctagaaatttttaccatttgtcttctgtgtagctgcaaggaacactaatgtttatacaactgtcagtccacccagtggtgcaactggttctgattcagtcttccgattcctttttatttttcactttttcctatttctgaatttttttttttatttgtgatcttgattttgatgaggggttggggagtggggagggagtcgaaccaagacttggagttaagaggattttcatcttttgcatccaacaggcagaatatgatctgtgtccaaaagtgaacttgagtcaggaatgaatcaatttcagcataaacaagcacaaaaatttagtctgctggctgactggaagcaaaaaagtcaagatggaatatgatgaattccaacacaatggggcaccaaggcctttaggcctctctttttattttgctttggttttgtttgtttttctttagagacatgctctttctcatgggacttgaagtggactcatctttgtgcagtgctggttttgccatactcatttcaagtattatagacatatgtaatggtgaaaatatatgaactgtggcctttttcattcttgttacttgtgatgcaattaagtgaagataagaaaaaaaaaaaaaaagcagagatttaccatgtatcagtgcctggctttttgttataaagctttgtttgtctagtgctcttttgctataaaatagactgtagtacaccctagtaggaaaaaaaaaaaactaaatttaaaaataaaaaatatatttggcttatttttcgcaggagcaatccttttataccatgaatattacaaaaaaattgtcagattctgaatatttcttctttgtagatttttggaatcattatgagtaaaagtttgttactttattttactatttaaaagatgttattttaccatgtgttaccaagatgaaactgtatgggtagcttttttgtttgttttttgttttgtttttgtttttgtttttgtttttagttgtaggtcgcagcggggaaattttttgcgactgtacacatagctgcagcattaaaaacttaaaaaaattgttaaaaaaaaaaaaagggaaaacatttcaaaaaaaaaaaaaaagataaacagttacaccttgttttcaatgtgtggctgagtgcctcgattttttcatgtttttggtgtatttctgatttgtagaagtgtccaaacaggttgtgtgctggagttccttcaagacaaaaacaaacccagcttggtcaaggccattacctgtttcccatctgtagttattcgatgaagtcatgtacatgaccgttctgtagcaataaatgtgccatttttataaactgtttctgacacttgtttcatttcattttgcattgtccatatagctatgattctcttctgtaagtaaaacgcatctatatttcattttccaagtgttggaggtattgacagcttaacaaacaaaacatacaaaaaaaatcacaaaaacaaattgaaaagcaaagcacatgattgatcaaggaagagatgcccttaatgaaaatggaacgggatgcatgcaaaacaaaaagaaaactgtctagaggattaactaattgaaggaatataattaatgtgtgtgtaacactgaagctatgcatttgaagagctctgaactgcaccagtgttttcggttgtgctgcaggttgctaagtcaagtcagccttaaccttttgcaccagttggtcggctgtttggcagaacattctcagatcttttcagtcaaaaatctaagatgatttattttgtatcactttgttaaaagctgaatattgttaactacagttaatattaacactgtatttatactttctcaaactacatccgccccaccacttctggttgcctctgttgactattaatccagatgtaaacaaccagatgtttttttctaacttgtacaaactgacgtgtgtcaactatcatggaaggaaaaaaatgtacagattaaaattattcagtgttatgtactgtaagttaatatttttgtagaatggacatcaatctactttgcaaaatttggaggctatttcaacattgcactgtagaaatgtaaagtaatgtatgcaatgtaaaggaaagcccgcggtagctgagcgcttcataacagaatgttctaatcaagtacgtggtatttggggatgtctccaatattgctcttgtattctttctaattgggtttagtgactagttgaaggaaaatgttataacgccatttggttcacatgtgaagtgccctccatagccaaatgttgggatttttttttttttcgtttttggttggactgtttgcagatatttaaattttatgaaatttccaaagattttggttgataacccccttttaccttctaaatgatttgagatgttcttatgttcttactgtgtgttttaaatatatataaaagagccacaagcattt

SEQ ID NO: 42 - protein, 671 aa, Homo sapiens
MHHQQRMAALGTDKELSDLLDFSAMFSPPVSSGKNGPTSLASGHFTGSNVEDRSSSGSWGNGGHPSPSRNYGDGTPYDHMTSRDLGSHDNLSPPFVNSRIQSKTERGSYSSYGRESNLQGCHQQSLLGGDMDMGNPGTLSPTKPGSQYYQYSSNNPRRRPLHSSAMEVQTKKVRKVPPGLPSSVYAPSASTADYNRDSPGYPSSKPATSTFPSSFFMQDGHHSSDPWSSSSGMNQPGYAGMLGNSSHIPQSSSYCSLHPHERLSYPSHSSADINSSLPPMSTFHRSGTNHYSTSSCTPPANGTDSIMANRGSGAAGSSQTGDALGKALASIYSPDHTNNSFSSNPSTPVGSPPSLSAGTAVWSRNGGQASSSPNYEGPLHSLQSRIEDRLERLDDAIHVLRNHAVGPSTAMPGGHGDMHGIIGPSHNGAMGGLGSGYGTGLLSANRHSLMVGTHREDGVALRGSHSLLPNQVPVPQLPVQSATSPDLNPPQDPYRGMPPGLQGQSVSSGSSEIKSDDEGDENLQDTKSSEDKKLDDDKKDIKSITRSRSSNNDDEDLTPEQKAEREKERRMANNARERLRVRDINEAFKELGRMVQLHLKSDKPQTKLLILHQAVAVILSLEQQVRERNLNPKAACLKRREEEKVSSEPPPLSLAGPHPGMGDASNHMGQM

SEQ ID NO: 43 - DNA, 3536 bp, Homo sapiens
agcgccggcgaggcgcgggaggaggagaagcagtggggaggcgcagccgctcacctgcggggcagggcgcggaggagggacccgggctgcgcgctctcgggccgaggaaccaggacgcgcccggagcctcgcacgcggccaagctcggggcgtcccctcccctcggccgggcgaactcaaggggcgcagctctttgctttgacagagctggccggcggaggcgtgcagagcggcgagccggcgagccaggctgagaaactcgagccgggaacaaagaggggtcggactgagtgtgtgtgtcggctcgagctccgggcagaggcatttgggcccgaggcccccgctgtgactccccgagactccgcagtgccctccactgcggagtccccgcgcttgccggcaaaaactttattcttggcaaacttctctttctcttcccctcctcctcggcccccatcttctgctcctcctccttctctagcagattaaatgagcctcgagaagaaaaaccgaagcgaaagggaagaaaataagaagatctaaaacggacatctccagcgtgggtggctcctttttctttttctttttttcccacccttcaggaagtggacgtttcgttatcttctgatccttgcaccttcttttggggcaaacggggcccttctgcccagatcccctctcttttctcggaaaacaaactactaagtcggcatccggggtaactacagtggagagggtttccgcggagacgcgccgccggaccctcctctgcactttggggaggcgtgctccctccagaaccggcgttctccgcgcgcaaatcccggcgacgcggggtcgcggggtggccgccggggcagcctcgtctagcgcgcgccgcgcagacgcccccggagtcgccagctaccgcagccctcgccgcccagtgcccttcggcctcgggggcgggcgcctgcgtcggtctccgcgaagcgggaaagcgcggcggccgccgggattcgggcgccgcggcagctgctccggctgccggccggcggccccgcgctcgcccgccccgcttccgcccgctgtcctgctgcacgaacccttccaactctcctttcctcccccacccttgagttacccctctgtctttcctgctgttgcgcgggtgctcccacagcggagcggagattacagagccgccgggatgccccaactctccggaggaggtggcggcggcgggggggacccggaactctgcgccacggacgagatgatccccttcaaggacgagggcgatcctcagaaggaaaagatcttcgccgagatcagtcatcccgaagaggaaggcgatttagctgacatcaagtcttccttggtgaacgagtctgaaatcatcccggccagcaacggacacgaggtggccagacaagcacaaacctctcaggagccctaccacgacaaggccagagaacaccccgatgacggaaagcatccagatggaggcctctacaacaagggaccctcctactcgagttattccgggtacataatgatgccaaatatgaataacgacccatacatgtcaaatggatctctttctccacccatcccgagaacatcaaataaagtgcccgtggtgcagccatcccatgcggtccatcctctcacccccctcatcacttacagtgacgagcacttttctccaggatcacacccgtcacacatcccatcagatgtcaactccaaacaaggcatgtccagacatcctccagctcctgatatccctactttttatcccttgtctccgggtggtgttggacagatcaccccacctcttggctggttttcccatcatatgattcccggtcctcctggtccccacacaactggcatccctcatccagctattgtaacacctcaggtcaaacaggaacatccccacactgacagtgacctaatgcacgtgaagcctcagcatgaacagagaaaggagcaggagccaaaaagacctcacattaagaagcctctgaatgcttttatgttatacatgaaagaaatgagagcgaatgtcgttgctgagtgtactctaaaagaaagtgcagctatcaaccagattcttggcagaaggtggcatgccctctcccgtgaagagcaggctaaatattatgaattagcacggaaagaaagacagctacatatgcagctttatccaggctggtctgcaagagacaattatggtaagaaaaagaagaggaagagagagaaactacaggaatctgcatcaggtacaggtccaagaatgacagctgcctacatctgaaacatggtggaaaacgaagctcattcccaacgtgcaaagccaaggcagcgaccccaggacctcttctggagatggaagcttgttgaaaacccagactgtctccacggcctgcccagtcgaccccaaaggaacactgacatcaattttaccctgaggtcactgctagagacgctgatccataaagacaatcactgccaacccctctttcgtctactgcaagagccaagttccaaaataaagcataaaaaggttttttaaaaggaaatgtaaaagcacatgagaatgctagcaggctgtggggcagctgagcagcttttctcccctcatatctgcgtgcacttcccagagcatcttgcatccaaacctgtaacctttcggcaaggacggtaacttggctgcatttgcctgtcatgcgcaactggagccagcaaccagcacatccatcagcaccccagtggaggagttcatggaagagttccctctttgtttctgcttcatttttctttcttttcttttctcctaaagcttttatttaacagtgcaaaaggatcgtttttttttgcttttttaaacttgaatttttttaatttacactttttagttttaattttcttgtatattttgctagctatgagcttttaaataaaattgaaagttctggaaaagtttgaaataatgacataaaaagaagccttctttttctgagacagcttgtctggtaagtggcttctctgtgaattgcctgtaacacatagtggcttctccgcccttgtaaggtgttcagtagagctaaataaatgtaatagccaaacccactctgttggtagcaattggcagccctatttcagtttattttttcttctgttttcttcttttctttttttaaacagtaaaccttaacagatgcgttcagcagactggtttgcagtgaattttcatttctttccttatcacccccttgttgtaaaaagcccagcacttgaattgttattactttaaatgttctgtatttgtatctgtttttattagccaattagtgggattttatgccagttgttaaaatgagcattgatgtacccattttttaaaaaagcaaggcacagcctttgcccaaaactgtcatcctaacgtttgtcattccagtttgagttaatgtgctgagcatttttttaaaagaagctttgtaataaaacatttttaaaaattgtcatttaaaaaaaaaaaaaaaaaa

SEQ ID NO: 44 - protein, 371 aa, Homo sapiens
MPQLSGGGGGGGGDPELCATDEMIPFKDEGDPQKEKIFAEISHPEEEGDLADIKSSLVNESEIIPASNGHEVARQAQTSQEPYHDKAREHPDDGKHPDGGLYNKGPSYSSYSGYIMMPNMNNDPYMSNGSLSPPIPRTSNKVPVVQPSHAVHPLTPLITYSDEHFSPGSHPSHIPSDVNSKQGMSRHPPAPDIPTFYPLSPGGVGQITPPLGWFSHHMIPGPPGPHTTGIPHPAIVTPQVKQEHPHTDSDLMHVKPQHEQRKEQEPKRPHIKKPLNAFMLYMKEMRANVVAECTLKESAAINQILGRRWHALSREEQAKYYELARKERQLHMQLYPGWSARDNYGKKKKRKREKLQESASGTGPRMTAAYI

(19) United States
(12) Patent Application Publication
(10) Pub. No.: US 2014/0037599 A1
(43) Pub. Date: Feb. 6, 2014

(54) Compositions and Methods of Treating T Cell Deficiency

(71) Applicant: The Trustees Of The University Of Pennsylvania, PHILADELPHIA, PA (US)

(72) Inventors: AVINASH BHANDOOLA, PHILADELPHIA, PA (US); ANTHONY W. S. CHI, PHILADELPHIA, PA (US); BRITTANY WEBER, PHILADELPHIA, PA (US)

(73) Assignee: THE TRUSTEES OF THE UNIVERSITY OF PENNSYLVANIA, PHILADELPHIA, PA (US)

(21) Appl. No.: 13/958,164

(22) Filed: Aug. 2, 2013

Related U.S. Application Data
(60) Provisional application No. 61/679,296, filed on Aug. 3, 2012.

(51) Int. Cl. C12N 5/0783

(52) U.S. Cl. CPC C12N 5/0636
USPC 424/93.21; 435/325; 506/9

(57) ABSTRACT

The invention provides compositions and methods for genetically modifying T cell progenitor cells (TCPC) to express TCF-1 to differentiate the TCPC, or its progeny, into a T cell. The invention also provides methods of using a T cell derived from a TCPC to treat a subject having a disease or disorder involving T cell deficiency.

### CROSS-REFERENCE TO RELATED APPLICATIONS

The present application claims priority to U.S. Provisional Application No. 61/679,296, filed Aug. 3, 2012, which is hereby incorporated by reference in its entirety herein.

### STATEMENT REGARDING FEDERALLY SPONSORED RESEARCH OR DEVELOPMENT

This invention was made with government support under Grant No. AI059621 awarded by the National Institutes of Health (NIH). The government has certain rights in the invention.

### BACKGROUND OF THE INVENTION

T cells develop within the thymus, and are essential for immune responses against many pathogens. There are many conditions in which T cell numbers diminish, including infection, advanced age, and following bone marrow transplantation. Thus, there is interest in achieving an understanding of the molecules regulating T cell commitment, specification, differentiation, and development, which allows for opportunities to modulate this process for therapeutic gain.

It was previously known that Notch signals within the thymic environment are involved in initiating T cell development. Within the thymus, Notch1 signals drive development through sequential steps during which alternative lineage potentials are lost and T-lineage-specific gene expression (specification) occurs (Schwarz et al., 2007, J. Immunol., 178: 2008-2017; Spangrude et al., 1990, J. Immunol., 145: 3661-3668; Doulatov et al., 2010, Nature Immunol., 11: 585-593; Rothenberg et al., 2010, Immunol. Rev., 238: 150-168). Although Notch is known to be necessary for early T-cell development, its downstream effectors have remained unclear (Pui et al., 1999, Immunity, 11: 299-308; Radtke et al., 1999, Immunity, 10: 547-558; Sambandam et al., 2005, Nature Immunol., 6: 663-670). Moreover, aberrant Notch signals have been shown to cause T cell leukemia, thereby limiting the use of Notch in gene therapy approaches to improve T cell commitment, specification, differentiation, and development.

Thus, there remains a need in the art for the compositions and methods to modulate T cell commitment, specification, differentiation, development, and reconstitution. The present invention satisfies these unmet needs.

### SUMMARY OF THE INVENTION

The invention relates to compositions and methods for genetically modifying a T cell progenitor cell (TCPC) to express at least one of TCF-1, TCF-3, TCF-4 or TCF-10 (a.k.a. LEF-1) to differentiate the TCPC, or its progeny, into a T cell. In one embodiment, the invention is a genetically modified T cell progenitor cell (TCPC) comprising a vector comprising a nucleic acid encoding at least one selected from the group consisting of T Cell Factor (TCF)-1, TCF-3, TCF-4 and TCF-10. In various embodiments, the genetically modified TCPC is at least one cell selected from the group consisting of an embryonic stem cell (ESC), an induced pluripotent stem cell (iPSC), a hematopoietic stem cell (HSC), a hematopoietic progenitor cell (HPC), a common lymphoid progenitor cell (CLP), an early lymphoid progenitor cell (ELP), an early thymic progenitor cell (ETP), a lymphoid-primed multipotent progenitor cell (LMPP) and a lineage marker-negative cell (LSK). In some embodiments, the genetically modified TCPC is stably transfected. In some embodiments where the genetically modified TCPC is stably transfected, the vector is at least one vector selected from the group consisting of a retroviral vector and a lentiviral vector. In other embodiments, the genetically modified TCPC is transiently transfected. In some embodiments where the genetically modified TCPC is transiently transfected, the vector is selected from the group consisting of a mRNA and a plasmid.

In one embodiment, the invention is a progeny cell derived from a genetically modified TCPC. In another embodiment, the invention is a T cell derived from a genetically modified TCPC. In some embodiments, the T cell derived from a genetically modified TCPC expresses at least one cell surface marker selected from the group consisting of CD2, CD3, CD25, CD4 and CD8.

In one embodiment, the invention is a method of deriving a T cell from a TCPC including the steps of contacting a TCPC with a vector comprising a nucleic acid encoding a polypeptide selected from the group consisting of T Cell Factor (TCF)-1, TCF-3, TCF-4 and TCF-10, allowing the vector comprising the nucleic acid encoding the polypeptide to enter the nucleus of the TCPC, allowing the nucleic acid encoding the polypeptide to be expressed in the TCPC, culturing the TCPC, isolating a progeny cell from the culture, detecting a T cell specific cell surface marker on the progeny cell, thereby deriving a T cell from a TCPC. In some embodiments, the nucleic acid encoding the polypeptide encodes TCF-1, where TCF-1 comprises the nucleic acid sequence of SEQ ID NO:37, or a modification thereof. In various embodiments, the TCPC is at least one cell selected from the group consisting of an embryonic stem cell (ESC), an induced pluripotent stem cell (iPSC), a hematopoietic stem cell (HSC), a hematopoietic progenitor cell (HPC), a common lymphoid progenitor cell (CLP), an early lymphoid progenitor cell (ELP), an early thymic progenitor cell (ETP), a lymphoid-primed multipotent progenitor cell (LMPP) and a lineage marker-negative cell (LSK). In some embodiments, the genetically modified TCPC is stably transfected. In some embodiments where the genetically modified TCPC is stably transfected, the vector is at least one vector selected from the group consisting of a retroviral vector and a lentiviral vector. In other embodiments, the genetically modified TCPC is transiently transfected. In some embodiments where the genetically modified TCPC is transiently transfected, the vector is selected from the group consisting of a mRNA and a plasmid. In one embodiment, the invention is a progeny cell derived from a genetically modified TCPC. In another embodiment, the invention is a T cell derived from a genetically modified TCPC. In some embodiments, the T cell derived from a genetically modified TCPC expresses at least one cell surface marker selected from the group consisting of CD2, CD3, CD25, CD4 and CD8.

In another embodiment, the invention is a method of treating a subject with a disease or disorder, including the step of administering to the subject at least one T cell derived from a genetically modified TCPC, where the genetically modified TCPC comprises a nucleic acid encoding at least one polypeptide selected from the group consisting of T Cell Factor (TCF)-1, TCF-3, TCF-4 and TCF-10. In one embodiment, the nucleic acid encoding the polypeptide encodes TCF-1 and where TCF-1 comprises the nucleic acid sequence of SEQ ID NO:37, or a modification thereof. In various embodiments, the genetically modified TCPC is at least one cell selected from the group consisting of an embryonic stem cell (ESC), an induced pluripotent stem cell (iPSC), a hematopoietic stem cell (HSC), a hematopoietic progenitor cell (HPC), a common lymphoid progenitor cell (CLP), an early lymphoid progenitor cell (ELP), an early thymic progenitor cell (ETP), a lymphoid-primed multipotent progenitor cell (LMPP) and a lineage marker-negative cell (LSK). In some embodiments, the genetically modified TCPC is stably transfected. In some embodiments where the genetically modified TCPC is stably transfected, the vector is at least one vector selected from the group consisting of a retroviral vector and a lentiviral vector. In other embodiments, the genetically modified TCPC is transiently transfected. In some embodiments where the genetically modified TCPC is transiently transfected, the vector is selected from the group consisting of a mRNA and a plasmid. In some embodiments, the T cell expresses at least one cell surface marker selected from the group consisting of CD2, CD3, CD25, CD4 and CD8. In some embodiments, the disease or disorder is a T cell deficiency. In some embodiments, the T cell deficiency is at least one selected from the group consisting of T cell deficiency following bone marrow ablation, T cell deficiency following bone marrow transplant, T cell deficiency following chemotherapy, and T cell deficiency following corticosteroid therapy.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIG. 1A through FIG. 1G, depicts the results of experiments demonstrating that TCF-1 is necessary for early T-lineage development and specification. FIG. 1A is a graph illustrating TCF-1 gene expression in bone marrow (BM), thymic progenitors and T-cells. Expression is shown relative to 18S RNA and lymphoid-primed multipotent progenitors (LMPP). CLP, common lymphoid progenitor. FIG. 1B is a set of graphs showing that mixed BM chimaeras were generated using TCF-1^{-/-} BM and wild-type BM. FIG. 1C is a graph illustrating the chimerism of TCF-1^{-/-} cells, normalized to hematopoietic stem cells (HSC) (four mice per group; three independent experiments), **P<0.005. FIG. 1D illustrates the results of experiments where TCF-1^{+/-} and TCF-1^{-/-} lineage marker-negative Sca1⁺ Kit⁺ (LSK) progenitors were seeded onto OP9 and OP9-DL1 stroma and analyzed for myeloid (Mac1⁺Gr1⁺) and T development (Thy1⁺CD25⁺). FIG. 1E is a set of graphs illustrating the cellularity of day 6 cultures, including B (CD19⁺). FIG. 1F illustrates the results of experiments where TCF-1^{-/-} and TCF-1^{+/+} LMPPs were seeded onto OP9-DL4 and lineage-negative cells from TCF-1^{+/+} and TCF-1^{-/-} cultures were harvested at day 4 for gene expression. The right side of panel corresponds to T lineage cells made from normal progenitors at day 4 in culture. Lineage-negative cells from these early cultures retain progenitor activity (Taghon et al., 2005, Genes Dev., 19:965-978). Heat map shows a selection of T-lineage genes with expression increased more than twofold compared with TCF-1^{+/+} lineage-negative cells and represents the log₂ value of normalized signal level. Rows represent two independent samples for each population. FIG. 1G is a set of graphs illustrating the QRT-PCR validation of selected genes. All error bars are means±s.e.m.

FIG. 2, comprising FIG. 2A through FIG. 2G, depicts the results of experiments demonstrating that ectopic expression of TCF-1 elicits T-lineage cells in vitro. FIG. 2A illustrates the results of experiments where wild-type LMPPs were transduced with control murine stem cell virus (MSCV) containing Violet-Excited GFP (MSCV-VEX) or MSCV containing human TCF-1 (MSCV-TCF-1-VEX). Transduced cells were isolated by cell sorting, and seeded onto OP9 or OP9-DL4. Plots are gated on VEX⁺CD45.2⁺ Mac1⁻Gr1⁻ cells, shown on day 12. FIG. 2B illustrates the results of experiments illustrating that, on OP9 stroma, TCF-1-expressing progenitors gave rise to myeloid cells (Mac1⁺Gr1⁺), shown on day 3, but TCF-1 inhibited the development of CD19⁺ B cells, shown on day 12. FIG. 2C illustrates the results of experiments where TCF-1-expressing Thy1⁺CD25⁺ cells were isolated from OP9 cultures after 8 days and injected intrathymically into congenic recipients. Shown is 19 days post injection. FIG. 2D illustrates the results of experiments where β-catenin^{f/f}MxCre⁺ and control mice were induced with poly(I:C) and LMPPs were isolated, transduced with MSCV-TCF-1-VEX, and 2,000 transduced cells were seeded per well on OP9 stroma and analysed at day 7, 10 and 12. Plots are gated on VEX⁺CD45.2⁺ Mac1⁻Gr1⁻ cells, shown on day 10. FIG. 2E is a graph illustrating the relative cellularity of day 7 cultures. Results represent triplicates±s.d. FIG. 2F illustrates the results of experiments where Notch1^{f/f}MxCre⁺Rosa^{YFP/+} mice were induced with poly(I:C) and YFP⁺ LSK progenitors were isolated and transduced with TCF-1 or vector control and injected intrathymically into sublethally irradiated recipients. Shown is day 10 analysis, two independent experiments, 4-6 mice per experiment. Frequency of donor-derived TCF-1-expressing Thy1⁺CD25⁺ cells compared to control, P=0.03. FIG. 2G illustrates the results of a limiting dilution analysis performed on TCF-1-expressing LMPPs grown on OP9 stroma; this was compared to LMPPs grown on OP9-DL4 stroma. Frequencies of lineage-competent cells were similar. (TCF-1-expressing Thy1⁺CD25⁺ lineage on OP9: 1 in 17 (95% confidence interval 1 in 12-26), control Thy1⁺CD25⁺ lineage on OP9-DL4: 1 in 23 (95% confidence interval 1 in 15-34).)

FIG. 3, comprising FIG. 3A through FIG. 3E, depicts the results of experiments demonstrating that TCF-1 upregulates expression of T-lineage specific genes. FIG. 3A illustrates the results of a microarray-based analysis of gene expression in TCF-1-expressing Thy1⁺CD25⁺ T cells on OP9, control Thy1⁺CD25⁺ on OP9-DL4, and LMPPs. Shown are selected T-lineage genes upregulated greater than twofold in TCF-1-expressing T-lineage cells. Scale represents the log₂ value of normalized signal level. TCF-1 1 and TCF-1 2 represent biological replicates. FIG. 3B are a set of graphs depicting the QRT-PCR validation of selected genes normalizing to GAPDH and LMPP. U.D., undetectable. FIG. 3C illustrates the results of experiments where chromatin immunoprecipitation (ChIP) was performed on double-negative (DN) thymocytes using TCF-1 or IgG antibodies. QRT-PCR was performed with primers flanking putative TCF-1-binding sites. Axin2 is a positive control and CD3ε negative control refers to region lacking TCF-1 binding sites. FIG. 3D depicts an illustration of ChIP as described. FIG. 3E is a graph illustrating that TCF-1 enhances TCF-1 promoter activity. 293T cells were cotransfected with the pGL3 vector containing the TCF-1 promoter and −1.3 kb TCF-1 binding site or a mutated TCF-1 binding site, and with either empty vector or MSCV-TCF-1. Luciferase activity is shown relative to Renilla and normalized to empty vector. All error bars are means±s.e.m. of triplicate samples. *P<0.05, **P<0.005.

FIG. 4, comprising FIG. 4A through FIG. 4C, depicts the results of experiments illustrating that TCF-1 is expressed in the earliest T cell progenitors and is downstream of Notch1. FIG. 4A is a graph illustrating the results of experiments where LMPPs from wild-type BM were seeded onto OP9-DL4 and Mac1⁻Gr1⁻ cells were harvested over a 5-day period. Relative gene expression of TCF-1 and the Notch target genes Ptcra and Deltex is shown after normalizing to 18S RNA and LMPP. FIG. 4B illustrates the TCF-1 locus with conserved putative CSL (for CBF1, Suppressor of Hairless, and Lag-1) binding sites. Further, FIG. 4B illustrates the results of ChIP on DN thymocytes using Notch1 or control IgG antibodies. QRT-PCR was performed with primers flanking putative CSL-binding sites. Shown is the relative percentage of input DNA. FIG. 4C illustrates the results of experiments where Scid.adh cells were treated with 1 μM gamma-secretase inhibitor (GSI, a pan-Notch inhibitor) or DMSO for 6 h in culture. Cells were subjected to ChIP analysis as in FIG. 4B. Shown is the relative percentage of input DNA in GSI- or DMSO-treated cultures. All error bars are means±s.e.m. of triplicate samples, *P<0.05, **P<0.005.

FIG. 5, comprising FIG. 5A through FIG. 5C, depicts the results of experiments illustrating that thymic development is aberrant in TCF-1 deficient mice. FIG. 5A is a graph depicting total thymic cellularity, comparing wild-type littermate control mice to TCF-1−/− mice. Mice were 4-6 weeks of age. FIG. 5B depicts representative flow plots and absolute numbers of early thymic progenitors (ETPs) (Lin-Kit+CD25−), DN2 cells (Lin-Kit+CD25+) and DN3 cells (Lin-Kit-CD25+). Results represent 4 or more mice/group, +/−s.e.m.*p<0.05, **p<0.005. FIG. 5C illustrates that TCF-1−/− thymocytes exhibit a partial block at the immature single positive (ISP) CD8+CD4−CD3ε− stage of development.

FIG. 6, comprising FIG. 6A through FIG. 6D, depicts the results of experiments illustrating the in vivo development of TCF-1 deficient progenitors. FIG. 6A illustrates the results of experiments where TCF-1+/+ and TCF-1−/− Lin-Sca-1+Kit+(LSK) cells were isolated and intrathymically injected into sublethally irradiated recipients and analyzed 10 days later. Shown is a representative example of the thymus of mice that received TCF-1+/+ or TCF-1−/− progenitors, analyzed for DN3 (Lin-Kit−CD25+) and myeloid (Mac1+Gr1+) cells. FIG. 6B is graph illustrating the absolute cell number of DN3 cells, result represents 3-4 mice per group+/−s.e.m., *p<0.05. FIG. 6C is a graph illustrating the relative expression of TCF-1 related family member, LEF-1 throughout T cell development. Results represent the relative gene expression compared to LMPP after normalizing to 18S RNA. Error bars are s.e.m. FIG. 6D is a graph illustrating the relative gene expression of LEF-1 and Notch1 in TCF-1+/+ and TCF-1−/− DN3 cells. Results represent averages of 2-4 mice per group, normalized to TCF-1+/+ DN3 cells. Error bars are s.e.m., *p<0.05.

FIG. 7, comprising FIG. 7A and FIG. 7B, depicts the results of experiments demonstrating that TCF-1, but not Bcl-xL, restores T cell development from TCF-1−/− progenitors in vitro. FIG. 7A illustrates the results of experiments where LSK progenitors from TCF-1−/− or TCF-1+/− mice were transduced with MCSV-Bcl-xL (GFP) or empty vector MCSV-GFP (MigR1) virus. Cells were seeded on OP9-DL4 in equal cell number. In this experiment, transduced cells were not isolated by a second round of cell sorting. Bcl-xL-expressing TCF-1−/− progenitors failed to undertake T-lineage development, shown on day 10. Plots at right are gated to be Mac1-negative and Gr1-negative. FIG. 7B illustrates the results of experiments where LSK progenitors were isolated from TCF-1−/− or TCF-1+/− mice, transduced with MCSV-VEX-control virus or MSCV-TCF-1-VEX Transduced cells were isolated by a second round of cell sorting, and then seeded onto OP9-DL4 for 10 days to assess T cell development. Shown is the gating strategy whereby first GFP+CD45+ hematopoietic cells are gated and myeloid lineage cells are excluded. T-lineage development is shown by Thy1 versus CD25 expression. Results were consistent at earlier and later time points. Data are representative of at least 3 independent experiments.

FIG. 8 depicts the results of experiments demonstrating that TCF-1−/− progenitors upregulate Notch1 gene targets but fail to upregulate T cell-specific genes. TCF-1−/− and TCF-1+/+ LMPPs were isolated by cell sorting and seeded onto OP9-DL4 stroma. After five days of culture, lineage negative cells (Mac1−Gr1−CD25−) and Thy1+CD25+ T lineage cells from TCF-1+/+ cells were harvested for RNA and cDNA synthesis. QRT-PCR analysis was performed on Notch1 targets, Deltex1 and Hes1, and T cell genes, Gata3 and CD3e. Shown is the relative expression compared to LMPP after normalizing to 18S RNA. Error bars are s.e.m.

FIG. 9, comprising FIG. 9A through FIG. 9E, depicts the results of experiments demonstrating that TCF-1 and Notch1 signals are additive in vitro and in vivo. FIG. 9A illustrates the results of experiments where TCF-1 or control expressing LSK progenitors were intrathymically injected into sublethally irradiated mice. Mice were analyzed on day 14 for thymic reconstitution (5 mice/group). FIG. 9B is a graph illustrating the absolute numbers of door derived thymocytes, **p<0.005. FIG. 9C illustrates the results of experiments where wild-type LSK progenitors were similarly isolated and transduced with MSCV-TCF-1-VEX or control vector. Transduced cells were isolated by cell sorting and equal numbers were seeded on OP9-DL4 and OP9. Development of Thy1+CD25+ cells is shown on day 12. FIG. 9D is a graph depicting the relative cellularity of cultures from day 12 analysis (4 wells/group), *p<0.05,**p<0.005. FIG. 9E is a set of graphs depicting the expression of T-lineage genes, from Thy1+CD25+ cells isolated from day 10 cultures. DN3 thymocytes are shown on the right for comparison. Results are relative to LSK after normalizing to GAPDH. Error bars are s.e.m.

FIG. 10, comprising FIG. 10A through FIG. 10E, depicts the results of experiments characterizing TCF-1-expressing Thy1+CD25+ cells. FIG. 10A illustrates the results of experiments where wild-type LMPPs were isolated and transduced with control MSCV-VEX or MSCV-TCF-1-VEX. Transduced cells were isolated by cell sorting, and seeded onto OP9 stroma. Plots are gated on VEX+CD45.2+Mac1−Gr1− cells. No Thy1+CD25+ cells were observed from control-expressing cells at all timepoints examined. FIG. 10B is graph depicting the relative cellularity of TCF-1-expressing Thy1+CD25+ cells cultured on OP9 stroma. FIG. 10C illustrates the characterization of cell surface markers on TCF-1-expressing Thy1+CD25+ cells after two weeks in culture. FIG. 10D illustrates the results of experiments where wild-type LSK progenitors were transduced with TCF-1 in the GFP (MigR1) or VEX retroviral constructs. Shown is the relative expression of human and mouse TCF-1 48 hours later, normalized to LSK progenitors transduced with empty vector control. Below, transduced progenitors were also seeded onto OP9 and shown is a day 7 analysis. FIG. 10E illustrates the results of experiments where TCF-1-expressing or control LSK progenitors were seeded in equal number in triplicate on OP9-DL4 or OP9 in the presence of 0.5 μm GSI or DMSO as control. Plots gated as described in FIG. 2A. Shown is day 12 analysis. Error bars are s.e.m.

FIG. 11, comprising FIG. 11A through FIG. 11D, depicts the results of experiments demonstrating that the development of TCF-1 expressing Thy1+CD25+ cells is independent of β-catenin. FIG. 11A illustrates the confirmation of β-catenin deletion by PCR of genomic DNA from β-cateninf/fMxCre+ and β-cateninf/fMxCre− Thy1+CD25+ cells isolated by cell sorting from day 10 cultures, performed as previously described (Brault et al., 2001, Development, 128: 1253-1264). FIG. 11B illustrates the results of experiments where wild-type LMPP progenitors were isolated and transduced with both MSCV-ICAT (GFP) and MSCV-TCF-1-VEX or MSCV-TCF-1-VEX alone. ICAT is a small molecule inhibitor of β-catenin that disrupts the ability of β-catenin to interact with TCF-1 (Hossain et al., 2008, Int. Immunol., 20: 925; Tago et al., 2000, Genes Dev., 14: 1741-1749). Transduced cells were isolated by a second round of cell sorting and seeded on OP9 stroma. Shown is a representative example of day 12 cultures. FIG. 11C illustrates that ICAT was functionally able to inhibit the β-catenin/TCF-1 mediated activation of the TCF-1 reporter, TOPFLASH, which contains a series of multimerized TCF-1/LEFT binding sites (van de Wetering, 1991, EMBO J., 10: 123-132). 293T cells were cotransfected with the TOPFLASH reporter, β-catenin^{ΔGSK}, and TCF-1 and with either empty vector or MSCV-ICAT. Luciferase activity is shown relative to Renilla and normalized to an empty vector control. Bars represent mean of triplicates+/−SD, *p=0.0003. Results are representative of 3 independent experiments. FIG. 11D depicts a schematic representation of ICAT-mediated inhibition of β-catenin-TCF-1 interactions.

FIG. 12, comprising FIG. 12A and FIG. 12B, depicts the results of experiments demonstrating that ectopic expression of TCF-1 is sufficient to give rise to T-lineage cells from CD150+ HSCs but not from myelo-erythroid progenitors. FIG. 12A illustrates the results of experiments where CD150+Lin-Sca1+Kit+Flt3− fetal liver HSCs were transduced with MSCV-TCF-1-VEX. VEX+ cells were isolated by cell sorting, then seeded on OP9 stromal cells. Shown is the development of Thy1+CD25+ T-lineage cells from day 14 cultures. Plots are gated on VEX+CD45+ hematopoietic cells. FIG. 12B illustrates the results of experiments where Lin-Sca1-Kit+(LK) myeloid progenitors or LSK progenitors from wild-type bone marrow were transduced with MSCV-TCF-1-VEX and seeded on OP9 for 10 days to assess the development of Thy1+CD25+ T-lineage cells. Plots are gated on VEX+CD45.2+ cells in culture. Thy1+CD25+ T-lineage cells are observed from LSK cultures whereas ectopic expression of TCF-1 in myeloid progenitors failed to upregulate surface expression of Thy1 and CD25.

FIG. 13 depicts the results of experiments demonstrating that ectopic expression of TCF-1 in progenitors in vivo does not cause T-cell leukemia. To determine whether ectopic expression of TCF-1 results in T-cell acute lymphoblastic leukemia (T-ALL) as observed with ectopic expression of intracellular Notch1 (ICN1), TCF-1 or ICN1-expressing LSK progenitors were intravenously transferred into lethally irradiated recipients. Mice were analyzed at various timepoints for the presence of T-ALL. A representative example at 8 weeks in spleen is shown. Plots are gated on donor derived CD45.2+VEX+(TCF-1) or CD45.2+GFP+(ICN1) splenocytes.

FIG. 14, comprising FIG. 14A and FIG. 14B, depicts the results of experiments demonstrating that B-cells expand in Notch1^{f/f}MxCre⁺Rosa^{YFP} control-expressing cells in the thymus. FIG. 14A illustrate the results of experiments where B-cell development was analyzed in the thymus after intrathymic injection of TCF-1 or control-expressing Notch1^{f/f}MxCre⁺Rosa^{YFP} progenitors. Notch1 deletion results in the expansion of B-cells in the thymus from control-expressing progenitors (Wilson et al., 2001, J. Exp. Med., 194: 1003). Ectopic expression of TCF-1 inhibited the development of B-cells, and only the TCF-1 negative (VEX negative) donor cells developed into B-cells. These data are consistent with the in vitro data (FIG. 2B) and suggest that TCF-1 is able to inhibit B-cell development in vivo and in vitro. FIG. 14B illustrates the confirmation of Notch1 deletion in Notch1^{f/f}MxCre⁺Rosa^{YFP} progenitors. Deletion of Notch1 was first confirmed via genomic PCR (Liu et al., 2011, J. Clin. Invest., 121: 800-808). For further confirmation, Notch1^{f/f}MxCre⁺Rosa^{YFP} TCF-1 and VEX-expressing progenitors were seeded on OP9-DL1 stroma, which signals progenitors through Notch2 in addition through Notch1. Prior work has shown that Notch2 signaling is sufficient to induce T-lineage commitment from Notch1−/− progenitors in vitro on OP9-DL1 (Besseyrias et al., 2007, J. Exp. Med., 204: 331). However, Notch2 does not drive T cell development in the thymus, likely because the relevant Notch ligands are not present (Koch et al., 2008, J. Exp. Med., 205: 2515). This approach allowed us to obtain Thy1+CD25+ cells from both Notch1^{f/f}MxCre⁺Rosa^{YFP} TCF-1 and control-expressing cells which were analyzed for Notch1 expression. Samples were normalized to GAPDH. Error bars are s.e.m.

FIG. 15 depicts the results of experiments demonstrating that TCF-1 expressing T-lineage cells from fetal liver progenitors express potential TCF-1 gene targets at comparable levels to DN3 thymocytes. CCR9+Lin-Sca1+Kit+Flt3+ lymphoid progenitors from fetal liver were retrovirally transduced with MCSV-TCF-1-VEX for 48 hours in a cytokine cocktail containing IL3, IL6, and SCF. VEX-expressing cells were obtained by cell sorting and seeded onto OP9 stromal cells. TCF-1-expressing Thy1+CD25+ T-lineage cells were harvested from day 10 cultures. QRT-PCR analysis was performed on T-lineage genes shown in FIG. 3B. Shown is the relative expression compared to LSK progenitors. Error bars are s.e.m.

FIG. 16 is a set of graphs depicting the results of experiments demonstrating that TCF-1 gene targets are induced within 48 hours of retroviral transduction. LSK progenitors from wild-type BM were transduced with MSCV-TCF-1-VEX or control virus, MSCV-VEX for 48 hours. Cells were harvested and cell sorted for VEX+ cells and RNA was made from equal numbers of control and TCF-1-expressing progenitors. QRT-PCR analysis was performed on potential TCF-1 gene targets. Shown is the relative expression of TCF-1 gene targets compared to control LSK after normalizing to 18S RNA. Error bars are s.e.m.

FIG. 17 depicts the results of experiments demonstrating that the −31 kb CSL binding site upstream of TCF-1 is ICN1 responsive in a reporter assay. The −28 kb and −31 kb CSL binding sites were cloned separately upstream into a pGL3 vector containing the SV40 promoter to determine if these CSL sites are responsive to activation by MSCV-ICN1. Consistent with the absence of Notch1 localization shown in the CHIP assays in FIG. 4B, activation of the −28 kb construct was not detected and therefore subsequent experiments focused on mutagenesis and analysis of the −31 kb CSL binding site. Genomic coordinates represent the entire sequence cloned into the vector and below is shown the mutagenesis of the CSL binding site. 293T cells were transiently cotransfected with the pGL3 SV40 promoter vector (200 ng/well) containing the wild-type −31 kb CSL binding site or a vector in which the CSL binding site was mutated and MSCV-ICN1 (100 ng/well). Data were analyzed by comparing Luciferase activity to Renilla activity and adjusted to the fold increase over empty vector. Error bars are s.e.m., *p<0.0001.

FIG. 18 depicts the results of experiments validating an inducible TCF-1-ER system. FIG. 18A depicts results showing that MCSV-TCF-1-ER activates a TCF-1 reporter in a dose dependent response to 4-hydroxytamoxifen (4-OHT). 293T cells were transfected with a TCF-1 reporter containing multimerized TCF-1/LEFT binding sites (TOPFLASH) and MSCV-TCF-1-ER in the presence of increasing doses of 4-OHT. Luciferase activity is shown relative to renilla and normalized to empty vector. Bars are means+/−s.e.m of triplicate samples. FIG. 18B depicts results showing MCSV-TCF-1-ER activates an integrated TCF-1 reporter (293T-OT) and this activity is reversed upon removal of 4-OHT. 293-OT cells containing an integrated series of TCF/LEF multimerized binding sites were transfected with a MSCV-TCF-1 ER or MSCV-TCF-1-GFP (MigR1, constitutively active) in the presence or absence of 4-OHT (Sum). 4-OHT was removed by washing triplicate wells at six or thirty hours prior to cell harvest. Luciferase activity is shown relative to renilla and normalized to empty vector. Bars are means+/−s.e.m of triplicate samples. Data demonstrate that TCF-1-ER activity is reversed within one day of removal of 4-OHT.

FIG. 19 depicts the results of experiments demonstrating that MSCV-TCF-1-ER rescues T cell development from TCF-1-deficient progenitors in the presence of 4-OHT. TCF-1-deficient Lin-Sca+Kit+(LSK) progenitors were isolated by cell sorting and transduced with MCSV-TCF-1 ER. Transduced cells were isolated by a second round of cell sorting, and seeded onto OP9-DL4 stroma in the presence or absence of 4-OHT (5 μm) and cytokines IL-7 (1 ng/ml) and Flt3-L (5 ng/ml). Cultures were analyzed thirteen days later. Plots are gated on GFP+CD45.2+Mac1− cells.

FIG. 20 depicts the results of experiments demonstrating that the loss of TCF-1 in DN2 and DN3 progenitors diverts progenitors to the myeloid fate in the presence of Notch1 signals. FIG. 20A depicts results showing that TCF-1-deficient LSKs were transduced with TCF-1-ER-GFP and seeded on OP9-DL1 in the presence of 5 um 4-OHT for two weeks. DN2 (CD44+CD25+) and DN3 (CD44-CD25+) progenitors were isolated by cell-sorting and replated back on OP9-DL1 stroma in the presence or absence of 4-OHT. Cultures were analyzed at day eight for T (Thy1+CD25+) and myeloid (Mac1+Gr1+) development. FIG. 20B depicts results showing cellularity of DN2 cultures, demonstrating the increase in myeloid cells as a function of dose. Results represent triplicates for each cell dose, error bars are S.D FIG. 20C depicts results showing DN3 cellularity. Only cell dose (1000) was performed, results represent duplicate wells, error bars are S.D.

FIG. 21 depicts the results of experiments showing that loss of TCF-1 results in presence of myeloid-lineage cells in vivo. FIG. 21A is a schematic of experimental protocol. TCF-1-deficient LSKs were transduced with TCF-1 ER-GFP or TCF-1-VEX and seeded on OP9-DL1 stroma in the presence of 5 μm 4-OHT. After three weeks, cultures were assessed and all hematopoietic cells consisted of Thy1+CD25+ T-lineage cells. Cells were isolated off from the OP9-DL1 stroma and intrathymically injected into sublethally irradiated congenic recipients. Due to tamoxifen toxicity in vivo, TCF-1-VEX− expressing Thy1+CD25+ T-lineage cells were utilized as a positive control and no mice received tamoxifen. Mice were analyzed at day eight (FIG. 21B). Shown is the donor reconstitution from both TCF-1-VEX and TCF-1-ER-GFP recipient mice. Plots on right were gated for CD4−CD8− donor cells. Only TCF-1-ER-GFP donor cells gave rise to a Mac1+ myeloid population suggesting that loss of TCF-1 diverts T cell progenitors both in vitro and in vivo.

FIG. 22 depicts the results of experiments consistent with the explanation that LEF-1 compensates when TCF-1 is withdrawn in vitro. TCF-1-deficient LSKs were transduced with TCF-1-ER-GFP and seeded on OP9-DL1 stroma in the presence of 5 μm 4-OHT for two weeks (FIG. 22A). Total cultures were passaged onto fresh OP9-DL1 stroma in the presence or absence of 4-OHT. Shown is day five cultures, plots are gated on CD45.2+GFP+ cells. Cultures were also analyzed at day 12, shown is the CD44 by CD25 profiles to distinguish DN2 and DN3. CD25 expression is also shown as a histogram the right (FIG. 22B). DN2 and DN3 progenitors were isolated by cell-sorting from cultures shown in (FIG. 22B) and RNA was extracted for subsequent cDNA synthesis. Results are normalized to Gapdh and DN2 cells from +4-OHT cultures (−ΔΔCT) (FIG. 22C). Error bars are S.D of triplicate wells.

FIG. 23 depicts the results of experiments showing enhanced lineage diversion when TCF-1 is withdrawn in the absence of LEF-1. TCF-1^{-/-}LEF-1^{F/F} VavCre⁺ (DKO) and TCF-1^{-/-}LEF1^{+/+} LSKs were isolated and transduced with TCF-1-ER and transduced cells were seeded on OP9-DL1 stroma in the presence of 5 μm 4-OHT for two weeks. DN2 (CD44+CD25+) and DN3 (CD44-CD25+) progenitors were isolated by cell-sorting and replated back on OP9-DL1 stroma in the presence or absence of 4-OHT (FIG. 23A). Analysis of DN2 cultures, shown at day five (FIG. 23B). Analysis of DN3 cultures, shown on day five (FIG. 23C). At the timepoint examined, only progenitors deficient for both LEF-1 and TCF-1 exhibited Mac1 upregulation which suggests that loss of LEF-1 enhances lineage diversion when TCF-1 is withdrawn from T cell progenitors.

### DETAILED DESCRIPTION

The invention relates to the discovery that the expression of T cell Factor-1 (TCF-1) promotes the differentiation of T cell progenitor cells (TCPC) into T cells that express T cell markers. Thus, the invention includes compositions and methods for genetically modifying a TCPC to express at least one of TCF-1, TCF-3, TCF-4 or TCF-10 to differentiate the TCPC, or its progeny, into a T cell. The TCPC useful in the compositions and methods of the invention include any totipotent, pluripotent, or multipotent cell type having the potential to differentiate into a T cell, including but not limited to, embryonic stem cells (ESC), induced pluripotent stem cells (iPSC), hematopoietic stem cells (HSC), hematopoietic progenitor cells (HPC), common lymphoid progenitor cells (CLP), early lymphoid progenitor cells (ELP), early thymic progenitor cells (ETP), lymphoid-primed multipotent progenitor cells (LMPP) and lineage marker-negative Sca1⁺ Kit⁺ cells (LSK).

It is an advantage of the present invention that the expression of at least one of TCF-1, TCF-3, TCF-4 or TCF-10 in TCPC leads to T cell differentiation without malignant transformation. In preferred embodiments, the TCPC is a human TCPC. In one embodiment, the invention includes a method of making a T cell derived from a TCPC through the expression of at least one of TCF-1, TCF-3, TCF-4 or TCF-10 (a.k.a. LEF-1). In other embodiments, the invention includes in vitro and ex vivo culture systems for deriving a T cell from a TCPC. In various embodiments, the invention includes methods of using a T cell, derived from a TCPC through the expression of at least one of TCF-1, TCF-3, TCF-4 or TCF-10 (a.k.a. LEF-1), to treat a subject having a disease or disorder, such as a disease or disorder involving T cell deficiency. In various embodiments, the diseases or disorders treatable by the methods of the invention include, but are not limited to, T cell deficiency following bone marrow ablation, T cell deficiency following bone marrow transplant, T cell deficiency following chemotherapy, and T cell deficiency following corticosteroid therapy.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

As used herein, “autologous” refers to a biological material derived from the same individual into whom the material will later be re-introduced.

As used herein, “allogeneic” refers to a biological material derived from a genetically different individual of the same species as the individual into whom the material will be introduced.

As used herein, the term “basal medium” refers to a solution of amino acids, vitamins, salts, and nutrients that is effective to support the growth of cells in culture, although normally these compounds will not support cell growth unless supplemented with additional compounds. The nutrients include a carbon source (e.g., a sugar such as glucose) that can be metabolized by the cells, as well as other compounds necessary for the cell's survival. These are compounds that the cells themselves cannot synthesize, due to the absence of one or more of the gene(s) that encode the protein(s) necessary to synthesize the compound (e.g., essential amino acids) or, with respect to compounds which the cells can synthesize, because of their particular developmental state the gene(s) encoding the necessary biosynthetic proteins are not being expressed as sufficient levels. A number of base media are known in the art of mammalian cell culture, such as Dulbecco's Modified Eagle Media (DMEM), Knockout-DMEM (KO-DMEM), and DMEM/F12, although any base medium that supports the growth of primate embryonic stem cells in a substantially undifferentiated state can be employed.

The terms “cells” and “population of cells” are used interchangeably and refer to a plurality of cells, i.e., more than one cell. The population may be a pure population comprising one cell type. Alternatively, the population may comprise more than one cell type. In the present invention, there is no limit on the number of cell types that a cell population may comprise.

The term “cell medium” as used herein, refers to a medium useful for culturing cells. An example of a cell medium is a medium comprising DMEM/F 12 Ham's, 10% fetal bovine serum, 100 U penicillin/100 μg streptomycin/0.25 μg Fungizone. Typically, the cell medium comprises a base medium, serum and an antibiotic/antimycotic. However, cells can be cultured with stromal cell medium without an antibiotic/antimycotic and supplemented with at least one growth factor. Preferably the growth factor is human epidermal growth factor (hEGF). The preferred concentration of hEGF is about 1-50 ng/ml, more preferably the concentration is about 5 ng/ml. The preferred base medium is DMEM/F12 (1:1). The preferred serum is fetal bovine serum (FBS) but other sera may be used including horse serum or human serum. Preferably up to 20% FBS will be added to the above media in order to support the growth of stromal cells. However, a defined medium could be used if the necessary growth factors, cytokines, and hormones in FBS for cell growth are identified and provided at appropriate concentrations in the growth medium. It is further recognized that additional components may be added to the culture medium. Such components include but are not limited to antibiotics, antimycotics, albumin, growth factors, amino acids, interleukin-2 (IL-2), insulin, IFN-γ, IL-4, IL-7, GM-CSF, IL-10, IL-12, IL-15, TGFβ, SCF, Flt3-L and TNF-α and other components known to the art for the culture of cells. Antibiotics which can be added into the medium include, but are not limited to, penicillin and streptomycin. The concentration of penicillin in the culture medium is about 10 to about 200 units per ml. The concentration of streptomycin in the culture medium is about 10 to about 200 μg/ml. However, the invention should in no way be construed to be limited to any one medium for culturing cells. Rather, any media capable of supporting cells in tissue culture may be used.

The term “differentiated cell” refers to a cell of a more specialized cell type derived from a cell of a less specialized cell type (e.g., a TCPC) in a cellular differentiation process.

“Differentiation medium” is used herein to refer to a cell growth medium comprising an additive or a lack of an additive such that a TCPC that is not fully differentiated, develops into a cell with some or all of the characteristics of a differentiated cell when incubated in the medium.

A “donor” is a subject used as a source of a biological material containing TCPC, such as for example, bone marrow, peripheral blood, and umbilical cord blood. A “recipient” is a subject which accepts a biological material, such as, by way of examples, TCPC, genetically modified TCPC, or differentiated progeny of TCPC. In autologous transfers, the donor and recipient are one and the same, i.e., syngeneic.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

As used herein, a “cell culture” refers to the maintenance or growth of one or more cells in vitro or ex vivo. Thus, for example, TCPC culture is one or more cells having the potential to differentiate into a T cell in a growth medium of some kind A “culture medium” or “growth medium” are used interchangeably herein to mean any substance or preparation used for sustaining or maintaining cells.

An “effective amount” or “therapeutically effective amount” of a composition is that amount of compound which is sufficient to provide a beneficial effect to the subject to which the composition is administered.

An “isolated cell” refers to a cell which has been separated from other components and/or cells which naturally accompany the isolated cell in a tissue or mammal.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

A “polynucleotide” means a single strand or parallel and anti-parallel strands of a nucleic acid. Thus, a polynucleotide may be either a single-stranded or a double-stranded nucleic acid. The term “nucleic acid” typically refers to large polynucleotides. The terms “nucleic acid” and “polynucleotide” and the like refer to at least two or more ribo- or deoxy-ribonucleic acid base pairs (nucleotides) that are linked through a phosphoester bond or equivalent. Nucleic acids include polynucleotides and polynucleotides. Nucleic acids include single, double or triplex, circular or linear, molecules. Exemplary nucleic acids include RNA, DNA, cDNA, genomic nucleic acid, naturally occurring and non naturally occurring nucleic acid, e.g., synthetic nucleic acid.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A “recombinant polypeptide” is one which is produced upon expression of a recombinant polynucleotide.

The term “transfected” when use in reference to a cell (e.g. a TCPC), means a genetic change in a cell following incorporation of an exogenous molecule, for example, a nucleic acid (e.g., a transgene) or protein into the cell. Thus, a “transfected” cell is a cell (or a progeny thereof) into which an exogenous molecule has been introduced by the hand of man.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof. Synthetic polypeptides can be synthesized, for example, using an automated polypeptide synthesizer.

The term “protein” typically refers to large polypeptides.

The term “peptide” typically refers to short polypeptides.

As used herein, the term “transgene” means an exogenous nucleic acid sequence which exogenous nucleic acid is encoded by a transgenic cell or mammal.

A “recombinant cell” is a cell that comprises a transgene. Such a cell may be a eukaryotic cell or a prokaryotic cell.

By the term “exogenous nucleic acid” is meant that the nucleic acid has been introduced into a cell or an animal using technology which has been developed for the purpose of facilitating the introduction of a nucleic acid into a cell or an animal.

As used herein, a “substantially purified” cell is a cell that is essentially free of other cell types. Thus, a substantially purified cell refers to a cell which has been purified from other cell types with which it is normally associated in its naturally-occurring state.

A “therapeutic” treatment is a treatment administered to a subject who exhibits a sign or symptom of disease or disorder, for the purpose of diminishing or eliminating the sign or symptom.

As used herein, “treating a disease or disorder” means reducing the frequency or severity with which a sign or symptom of the disease or disorder is experienced by a patient.

The phrase “therapeutically effective amount,” as used herein, refers to an amount that is sufficient or effective to prevent or treat (delay or prevent the onset of, prevent the progression of, inhibit, decrease or reverse) a disease or disorder.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Description

The invention relates to the discovery that a TCPC can be differentiated into a T cell exhibiting at least one T cell marker through the expression of at least one of TCF-1, TCF-3, TCF-4 or TCF-10 (a.k.a. LEF-1). In various embodiments, the derived T cell exhibits at least one T cell marker selected from the group consisting of CD2, CD3, CD25, CD4 and CD8. Thus, the invention relates to compositions and methods for genetically modifying a TCPC to express at least one of TCF-1, TCF-3, TCF-4 or TCF-10 (a.k.a. LEF-1), as well as to culture systems for deriving T cells from a genetically modified TCPC. The invention also relates to methods of using T cells derived from a genetically modified TCPC to treat a subject having a disease or disorder involving T cell deficiency, including, but not limited to, T cell deficiency following bone marrow ablation, T cell deficiency following bone marrow transplant, T cell deficiency following chemotherapy, and T cell deficiency following corticosteroid therapy.

The invention provides, among other things, TCPC genetically modified to express at least one of TCF-1, TCF-3, TCF-4 or TCF-10 (a.k.a. LEF-1), and the differentiated progeny of the genetically modified TCPC. Such TCPC are characterized by various features, including, for example, the presence or absence of various phenotypic markers, the ability to undergo cell division within a given time period in a suitable growth medium, the ability to produce certain proteins, and a characteristic morphology. Non-limiting exemplary cell medium are a liquid medium such as DMEM or RPMI. Other suitable medium for TCPC cell maintenance, growth and proliferation would be known to the skilled artisan. Such media can include one or more of supplements, such as albumin, essential amino acids, non-essential amino acids, L-glutamine, a hormone, vitamins, interleukin-2 (IL-2), insulin, IFN-γ, IL-4, IL-7, GM-CSF, IL-10, IL-12, IL-15, TGFβ, SCF, Flt3-L and TNF-α, etc.

The invention therefore also provides cells differentiated with respect to the genetically modified TCPC, wherein the cells are the progeny of a genetically modified TCPC. A “progeny” of a genetically modified TCPC refers to any and all cells derived from a genetically modified TCPC as a result of clonal proliferation or differentiation. A “developmental intermediate” cell refers to any cell that is more differentiated then the genetically modified TCPC, but less differentiated that the fully differentiated T cell.

In a population or plurality of TCPC, or in a culture of TCPC, a majority of cells, but not all cells present, may or may not express a particular phenotypic marker indicative of a TCPC. In various embodiments, the TCPC population or culture of TCPC include cells in which greater than about 50%, 60%, 70%, 80%, 90%-95% or more (e.g., 96%, 97%, 98%, etc. . . . 100%) of the cells express a particular phenotypic marker. In particular aspects, 75%, 80%, 85%, 90%, 95% or more of the TCPC population or culture of TCPC express a particular phenotypic marker. In various embodiments, an TCPC population or culture of TCPC include cells in which less than about 25%, 20%, 15%, 10%, 5% or less (e.g., 4%, 4%, 2%, 1%) of the cells express a particular phenotypic marker. In various aspects, in a population of TCPC or a culture of TCPC, 25%, 20%, 15%, 10%, 5% or less (e.g., 4%, 3%, 2%, 1%) of the cells express a particular phenotypic marker.

Genetically modified TCPC cells of the invention (or progeny thereof) include co-cultures and mixed populations. Such co-cultures and mixed cell populations of cells include a first mammalian (e.g., a human TCPC) cell, and a second cell distinct from the first cell. A second cell can comprise a population of cells. Non-limiting examples of exemplary cells distinct from mammalian (e.g., a human TCPC) cell include a B cell, T cell, dendritic cell, NK cell, monocyte, macrophage or PBMCs. Additional non-limiting examples of exemplary cells distinct from mammalian (e.g., a human TCPC) cell include different adult or embryonic stem cells; totipotent, pluripotent or multipotent stem cell or progenitor or precursor cells; cord blood stem cells; placental stem cells; bone marrow stem cells; amniotic fluid stem cells; circulating peripheral blood stem cells; mesenchymal stem cells; germinal stem cells; reprogrammed stem cells; induced pluripotent stem cells; and differentiated cells.

The presence or absence of a given phenotypic marker can be determined using the methods disclosed elsewhere herein. Thus, the presence or absence of a given phenotypic marker can be determined by an antibody that binds to the marker. Accordingly, marker expression can be determined by an antibody that binds to each of the respective markers, in order to indicate which or how many TCPC are present in a given population or culture of TCPC express the marker. Additional methods of detecting these and other phenotypic markers are known to one of skill in the art.

Cell cultures of TCPC can take on a variety of formats. For instance, an “adherent culture” refers to a culture in which cells in contact with a suitable growth medium are present, and can be viable or proliferate while adhered to a substrate. Likewise, a “continuous flow culture” refers to the cultivation of cells in a continuous flow of fresh medium to maintain cell viability, e.g. growth.

In one embodiment, the invention includes a culture system comprising at least one T cell derived from a genetically modified TCPC expressing at least one of TCF-1, TCF-3, TCF-4 or TCF-10 (a.k.a. LEF-1). The culture system of the invention can include any kind of substrate, surface, scaffold or container known in the art useful for culturing cells. Non-limiting examples of such containers include cell culture plates, dishes and flasks. Other suitable substrates, surfaces and containers are described in Culture of Animal Cells: a manual of basic techniques (3rd edition), 1994, R. I. Freshney (ed.), Wiley-Liss, Inc.; Cells: a laboratory manual (vol. 1), 1998, D. L. Spector, R.D. Goldman, L. A. Leinwand (eds.), Cold Spring Harbor Laboratory Press; Embryonic Stem Cells, 2007, J. R. Masters, B. O. Palsson and J. A. Thomson (eds.), Springer; Stem Cell Culture, 2008, J. P. Mather (ed.) Elsevier; and Animal Cells: culture and media, 1994, D. C. Darling, S. J. Morgan John Wiley and Sons, Ltd. In some embodiments, the culture system comprises a two-dimensional scaffold. In other embodiments, the culture system comprises a three-dimensional scaffold. In one particular embodiment, the culture comprises a thymic organ culture, such as those described in Schmitt and Zúñiga-Pflücker, 2006, Immunol Rev. 209:95-102. By way of one example, a two dimensional OP9/OP9-DL co-culture system has become a widely used and invaluable tool in early T cell differentiation. The OP9 cell line is derived from the op/op mouse, which carries a mutation in the macrophage colony-stimulating factor (M-SCF) gene. The presence of M-CSF inhibits differentiation of blood lineages, other than macrophages. Thus, the absence of this factor on the stromal support system allows the study of erythroid, myeloid, and lymphoid differentiation. Under normal conditions (without ectopic expression of TCF-1), OP9 stromal cells support the development of B-lymphoid and myeloid lineage cells, but not T-cells. However, prior studies have demonstrated that OP9 stromal cell that ectopically express the Notch ligand, DL-1 or DL-4, promote T cell differentiation (Zúñiga-Pflücker, 2007, Curr Opin Immunol 19:163-168). Therefore, the OP9 stroma cell culture system is a powerful in vitro tool that allows an investigator to expand TCF-1 expressing T-cells prior to therapeutic use.

Genetically modified TCPC expressing at least one of TCF-1, TCF-3, TCF-4 or TCF-10 and their progeny include individual cells, and populations of cells, that are isolated or purified. As used herein, the terms “isolated” or “purified” refers to made or altered “by the hand of man” from the natural state (i.e., when it has been removed or separated from one or more components of the original natural in vivo environment.) An isolated composition can but need not be substantially separated from other biological components of the organism in which the composition naturally occurs. An example of an isolated cell would be a TCPC obtained from a subject such as a human. “Isolated” also refers to a composition, for example, a TCPC separated from one or more contaminants (i.e., materials and substances that differ from the cell). A population or culture of genetically modified TCPC (or their progeny) is typically substantially free of cells and materials with which it is be associated in nature. The term “purified” refers to a composition free of many, most or all of the materials with which it typically associates with in nature. Thus, a TCPC or its progeny is considered to be substantially purified when separated from other tissue components. Purified therefore does not require absolute purity. Furthermore, a “purified” composition can be combined with one or more other molecules. Thus, the term “purified” does not exclude combinations of compositions. Purified can be at least about 50%, 60% or more by numbers or by mass. Purity can also be about 70% or 80% or more, and can be greater, for example, 90% or more. Purity can be less, for example, in a pharmaceutical carrier the amount of a cells or molecule by weight % can be less than 50% or 60% of the mass by weight, but the relative proportion of the cells or molecule compared to other components with which it is normally associated with in nature will be greater. Purity of a population or composition of cells can be assessed by appropriate methods that would be known to the skilled artisan.

A primary isolate of a TCPC useful in the compositions and methods of the invention can originate from or be derived from, by way of non-limiting examples, peripheral blood, bone marrow and umbilical cord blood. Progeny of primary isolate TCPC, which include all descendants of the first, second, third and any and all subsequent generations and cells taken or obtained from a primary isolate, can be obtained from a primary isolate or subsequent expansion of a primary isolate. Subsequent expansion results in progeny of TCPC that can in turn comprise the populations or pluralities of TCPC, the cultures of TCPC, progeny of TCPC, co-cultures, etc. Thus, the genetically modified TCPC of the invention refers to a cell from a primary isolate, and any progeny cell therefrom. Accordingly, the genetically modified TCPC are not limited to those from a primary isolate, but can be any subsequent progeny thereof provided that the cell has the desired phenotypic markers, doubling time, or any other characteristic feature set forth herein.

### Genetic Modification

In some embodiments, nucleic acid encoding at least one of TCF-1, TCF-3, TCF-4 or TCF-10 is delivered into a TCPC using a retroviral or lentiviral vector. Retroviral and lentiviral vectors can be delivered into different types of eukaryotic cells as well as into tissues and whole organisms using transduced cells as carriers or cell-free local or systemic delivery of encapsulated, bound or naked vectors. The method used can be for any purpose where stable expression is required or sufficient. In other embodiments, the nucleic acid encoding at least one of TCF-1, TCF-3, TCF-4 or TCF-10 is delivered into TCPC using in vitro transcribed mRNA. In vitro transcribed mRNA can be delivered into different types of eukaryotic cells as well as into tissues and whole organisms using transfected cells as carriers or cell-free local or systemic delivery of encapsulated, bound or naked mRNA. The method used can be for any purpose where transient expression is required or sufficient.

In the context of gene therapy, the genetically modified TCPC expressing at least one of TCF-1, TCF-3, TCF-4 or TCF-10 (a.k.a. LEF-1), and their progeny, can be genetically modified to stably or transiently express at least a fragment of at least one of TCF-1, TCF-3, TCF-4 or TCF-10 (a.k.a. LEF-1). Accordingly, the invention provides the use of genetically modified TCPC and their progeny that have been cultured according to the methods of the invention. In one embodiment, the genetic modification results in the expression of a transgene or in a change of expression of an endogenous gene. Genetic modification may also include at least a second transgene. A second transgene may encode, for instance, a selectable antibiotic-resistance gene, a suicide gene, or another selectable marker.

In some embodiments, the genetically modified TCPC (and their progeny) include those transfected with a nucleic acid encoding at least one of TCF-1, TCF-3, TCF-4 or TCF-10 (a.k.a. LEF-1). The cells of the invention may be genetically modified using any method known to the skilled artisan. See, for instance, Sambrook et al. (2012, Molecular Cloning: A Laboratory Manual, Cold Spring Harbor Laboratory Press, Cold Spring Harbor, N.Y.), and in Ausubel et al., Eds, (1997, Current Protocols in Molecular Biology, John Wiley & Sons, New York, N.Y.). For example, a cell may be exposed to an expression vector comprising a nucleic acid including a transgene, such that the nucleic acid is introduced into the cell under conditions appropriate for the transgene to be expressed within the cell. The transgene generally is an expression cassette, including a polynucleotide operably linked to a suitable promoter.

Nucleic acids can be produced using various standard cloning and chemical synthesis techniques. Techniques include, but are not limited to nucleic acid amplification, e.g., polymerase chain reaction (PCR), with genomic DNA or cDNA targets using primers (e.g., a degenerate primer mixture) capable of annealing to antibody encoding sequence. Nucleic acids can also be produced by chemical synthesis (e.g., solid phase phosphoramidite synthesis) or transcription from a gene. The sequences produced can then be translated in vitro, or cloned into a plasmid and propagated and then expressed in a cell (e.g., a host cell such as yeast or bacteria, a eukaryote such as an animal or mammalian cell or in a plant).

Nucleic acids can be included within vectors as cell transfection typically employs a vector. The term “vector,” refers to, e.g., a plasmid, virus, such as a viral vector, or other vehicle known in the art that can be manipulated by insertion or incorporation of a polynucleotide, for genetic manipulation (i.e., “cloning vectors”), or can be used to transcribe or translate the inserted polynucleotide (i.e., “expression vectors”). Such vectors are useful for introducing polynucleotides in operable linkage with a nucleic acid, and expressing the transcribed encoded protein in cells in vitro, ex vivo or in vivo.

In various embodiments, the vector contains control elements, including expression control elements, to facilitate transcription and translation. The term “control element” is intended to include, at a minimum, one or more components whose presence can influence expression, and can include components other than or in addition to promoters or enhancers, for example, leader sequences and fusion partner sequences, internal ribosome binding sites (IRES) elements for the creation of multigene, or polycistronic, messages, splicing signal for introns, maintenance of the correct reading frame of the gene to permit in-frame translation of mRNA, polyadenylation signal to provide proper polyadenylation of the transcript of a gene of interest, stop codons, among others.

The present invention includes retroviral and lentiviral vectors comprising a nucleic acid encoding at least one of TCF-1, TCF-3, TCF-4 or TCF-10 that can be directly transduced into a TCPC. The present invention also includes an RNA construct encoding at least one of TCF-1, TCF-3, TCF-4 or TCF-10 that can be directly transfected into a TCPC. A method for generating RNA for use in transfection involves in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the TCF-1 gene to be expressed, and a polyA tail, typically 50-2000 bases in length.

The present invention includes vectors in which a nucleic acid encoding at least one of TCF-1, TCF-3, TCF-4 or TCF-10 is inserted. Vectors derived from retroviruses, such as the lentivirus, are suitable tools to achieve stable, long-term gene transfer since they allow long-term, stable integration of a transgene and its propagation into progeny cells. Lentiviral vectors have the added advantage over vectors derived from onco-retroviruses such as murine leukemia viruses in that they can transduce non-proliferating cells.

Vectors included are those based on viral vectors, such as retroviral (lentivirus for infecting dividing as well as non-dividing cells), foamy viruses (U.S. Pat. Nos. 5,624,820, 5,693,508, 5,665,577, 6,013,516 and 5,674,703; WO92/05266 and WO92/14829), adenovirus (U.S. Pat. Nos. 5,700,470, 5,731,172 and 5,928,944), adeno-associated virus (AAV) (U.S. Pat. No. 5,604,090), herpes simplex virus vectors (U.S. Pat. No. 5,501,979), cytomegalovirus (CMV) based vectors (U.S. Pat. No. 5,561,063), reovirus, rotavirus genomes, simian virus 40 (SV40) or papilloma virus (Cone et al., Proc. Natl. Acad. Sci. USA 81:6349 (1984); Eukaryotic Viral Vectors, Cold Spring Harbor Laboratory, Gluzman ed., 1982; Sarver et al., Mol. Cell. Biol. 1:486 (1981); U.S. Pat. No. 5,719,054). Adenovirus efficiently infects slowly replicating and/or terminally differentiated cells and can be used to target slowly replicating and/or terminally differentiated cells. Simian virus 40 (SV40) and bovine papilloma virus (BPV) have the ability to replicate as extra-chromosomal elements (Eukaryotic Viral Vectors, Cold Spring Harbor Laboratory, Gluzman ed., 1982; Sarver et al., Mol. Cell. Biol. 1:486 (1981)). Additional viral vectors useful for expression include reovirus, parvovirus, Norwalk virus, coronaviruses, paramyxo- and rhabdoviruses, togavirus (e.g., sindbis virus and semliki forest virus) and vesicular stomatitis virus (VSV) for introducing and directing expression of a polynucleotide or transgene in TCPC or progeny thereof (e.g., differentiated cells).

Vectors including a nucleic acid can be expressed when the nucleic acid is operably linked to an expression control element. As used herein, the term “operably linked” refers to a physical or a functional relationship between the elements referred to that permit them to operate in their intended fashion. Thus, an expression control element “operably linked” to a nucleic acid means that the control element modulates nucleic acid transcription and as appropriate, translation of the transcript.

The term “expression control element” refers to nucleic acid that influences expression of an operably linked nucleic acid. Promoters and enhancers are particular non-limiting examples of expression control elements. A “promoter sequence” is a DNA regulatory region capable of initiating transcription of a downstream (3′ direction) sequence. The promoter sequence includes nucleotides that facilitate transcription initiation. Enhancers also regulate gene expression, but can function at a distance from the transcription start site of the gene to which it is operably linked. Enhancers function at either 5′ or 3′ ends of the gene, as well as within the gene (e.g., in introns or coding sequences). Additional expression control elements include leader sequences and fusion partner sequences, internal ribosome binding sites (IRES) elements for the creation of multigene, or polycistronic, messages, splicing signal for introns, maintenance of the correct reading frame of the gene to permit in-frame translation of mRNA, polyadenylation signal to provide proper polyadenylation of the transcript of interest, and stop codons.

Expression control elements include “constitutive” elements in which transcription of an operably linked nucleic acid occurs without the presence of a signal or stimuli. For expression in mammalian cells, constitutive promoters of viral or other origins may be used. For example, SV40, or viral long terminal repeats (LTRs) and the like, or inducible promoters derived from the genome of mammalian cells (e.g., metallothionein HA promoter; heat shock promoter, steroid/thyroid hormone/retinoic acid response elements) or from mammalian viruses (e.g., the adenovirus late promoter; mouse mammary tumor virus LTR) are used.

Expression control elements that confer expression, or activity, in response to a signal or stimuli, which either increase or decrease expression, or activity, of operably linked nucleic acid or its expression product (i.e., mRNA, polypeptide), are “regulatable.” A regulatable element that increases expression, or activity, of an operably linked nucleic acid, or its expression product (i.e., mRNA, polypeptide), in response to a signal or stimuli is referred to as an “inducible element.” A regulatable element that decreases expression, or activity, of the operably linked nucleic acid, or its expression product (i.e., mRNA, polypeptide), in response to a signal or stimuli is referred to as a “repressible element” (i.e., the signal decreases expression; when the signal is removed or absent, expression is increased). In a particular exemplary embodiment, the regulatable element is estrogen receptor (ER) that coupled to at least one of TCF-1, TCF-3, TCF-4 or TCF-10 (a.k.a. LEF-1). In the presence of tamoxifen, or 4-OHT, the ER coupled to at least one of TCF-1, TCF-3, TCF-4 or TCF-10 translocates to the nucleus where the at least one of TCF-1, TCF-3, TCF-4 or TCF-10 is active. In the absence of tamoxifen, or 4-OHT, the ER coupled to at least one of TCF-1, TCF-3, TCF-4 or TCF-10 remains in the cytoplasm where the at least one of TCF-1, TCF-3, TCF-4 or TCF-10 is inactive. Such a regulatable system allows for the activation and deactivation the activity of at least one of TCF-1, TCF-3, TCF-4 or TCF-10 (a.k.a. LEF-1). In one non-limiting exemplary embodiment, such a regulatable system permits the activation of at least one of TCF-1, TCF-3, TCF-4 or TCF-10 in genetically modified TCPC while the TCPC are outside the patient, and permits the inactivation of at least one of TCF-1, TCF-3, TCF-4 or TCF-10 in genetically modified TCPC while the TCPC are inside the patient.

In some embodiments, expression control elements include elements active in a particular tissue or cell type, referred to as “tissue-specific expression control elements.” Tissue-specific expression control elements are typically more active in a specific cell or tissue types because they are recognized by transcriptional activator proteins, or other transcription regulators active in the specific cell or tissue type, as compared to other cell or tissue types.

In accordance with the invention, there are provided TCPC and their progeny transiently or stably transfected with a nucleic acid encoding at least one of TCF-1, TCF-3, TCF-4 or TCF-10 (a.k.a. LEF-1), or vector comprising a nucleic acid encoding at least one of TCF-1, TCF-3, TCF-4 or TCF-10 (a.k.a. LEF-1). Such transfected cells include but are not limited to a primary TCPC isolate, populations of TCPC, cell cultures of TCPC (e.g., passaged, established or immortalized cell line), as well as progeny cells thereof (e.g., a progeny of a transfected cell that is clonal with respect to the parent cell, or has acquired a marker or other characteristic of differentiation).

The nucleic acid or protein can be stably or transiently transfected (expressed) in the TCPC and the progeny thereof. The cell(s) can be propagated and the introduced nucleic acid transcribed, and protein expressed. A progeny of a transfected cell may not be identical to the parent cell, because there may be phenotypic changes occurring due to differentiation.

In various embodiments, the viral and non-viral vector systems useful for delivering protein encoding nucleic acid into a TCPC are deployed in in vitro, in vivo or ex vivo methods. The introduction of protein encoding nucleic acid into TCPC target cells can be carried out using a variety of methods known in the art, including osmotic shock (e.g., calcium phosphate), electroporation, microinjection, cell fusion, viral infection, vector transduction, etc. Introduction of nucleic acid in vitro, ex vivo or in vivo can also be accomplished using other techniques. For example, through the use of a polymeric substance, such as polyesters, polyamine acids, hydrogel, polyvinyl pyrrolidone, ethylene-vinylacetate, methylcellulose, carboxymethylcellulose, protamine sulfate, or lactide/glycolide copolymers, polylactide/glycolide copolymers, or ethylenevinylacetate copolymers. A nucleic acid can be entrapped in microcapsules prepared by coacervation techniques or by interfacial polymerization, for example, by the use of hydroxymethylcellulose or gelatin-microcapsules, or poly (methylmethacrolate) microcapsules, respectively, or in a colloid system. Colloidal dispersion systems include macromolecule complexes, nano-capsules, microspheres, beads, and lipid-based systems, including oil-in-water emulsions, micelles, mixed micelles, and liposomes.

Liposomes for introducing various compositions into cells are known in the art and include, for example, phosphatidylcholine, phosphatidylserine, lipofectin and DOTAP (e.g., U.S. Pat. Nos. 4,844,904, 5,000,959, 4,863,740, and 4,975,282; and GIBCO-BRL, Gaithersburg, Md.). piperazine based amphilic cationic lipids useful for gene therapy also are known (see, e.g., U.S. Pat. No. 5,861,397). Cationic lipid systems also are known (see, e.g., U.S. Pat. No. 5,459,127). Polymeric substances, microcapsules and colloidal dispersion systems such as liposomes are collectively referred to herein as “vesicles.”

### Methods

The invention includes methods of producing genetically modified TCPC expressing at least one of TCF-1, TCF-3, TCF-4 or TCF-10 (a.k.a. LEF-1), and their differentiated progeny. In various embodiments, the differentiated progeny express at least one T cell marker selected from the group consisting of CD2, CD3, CD25, CD4 and CD8. The invention also includes methods of administering genetically modified TCPC expressing at least one of TCF-1, TCF-3, TCF-4 or TCF-10 (a.k.a. LEF-1), and/or their differentiated progeny to a subject having a disease or disorder. In various embodiments, the diseases or disorders treatable by the methods of the invention include, but are not limited to, T cell deficiency following bone marrow ablation, T cell deficiency following bone marrow transplant, T cell deficiency following chemotherapy, and T cell deficiency following corticosteroid therapy.

In various embodiments, the methods of deriving a T cell from a TCPC include the steps of: contacting the TCPC with a vector comprising a nucleic acid encoding at least one of TCF-1, TCF-3, TCF-4 or TCF-10 (a.k.a. LEF-1), allowing the vector comprising the nucleic acid encoding at least one of TCF-1, TCF-3, TCF-4 or TCF-10 to enter the nucleus of the TCPC, allowing the nucleic acid encoding at least one of TCF-1, TCF-3, TCF-4 or TCF-10 to be expressed in the TCPC, culturing the TCPC, isolating a progeny cell from the culture, detecting a T cell specific cell surface marker on the progeny cell, thereby deriving a T cell from a TCPC. In some embodiments, the nucleic acid encoding TCF-1 comprises the nucleic acid sequence of SEQ ID NO:37, or a modification thereof. In some embodiments, the nucleic acid encoding TCF-3 comprises the nucleic acid sequence of SEQ ID NO:39, or a modification thereof. In some embodiments, the nucleic acid encoding TCF-4 comprises the nucleic acid sequence of SEQ ID NO:41, or a modification thereof. In some embodiments, the nucleic acid encoding TCF-10 comprises the nucleic acid sequence of SEQ ID NO:43, or a modification thereof. In various embodiments, the TCPC useful in the method of deriving a T cell includes at least one of an ESC, an iPSC, a HSC, a HPC, a CLP, an ELP, an ETP, an LMPP and a lineage marker-negative cell, such as an LSK. In some embodiments, the TCPC is stably transfected with nucleic acid encoding at least one of TCF-1, TCF-3, TCF-4 or TCF-10 (a.k.a. LEF-1), while in other embodiments the TCPC is transiently transfected. In various embodiments, the T cell derived by the methods of the invention expresses at least one of CD2, CD3, CD25, CD4 and CD8.

TCPC of the invention and their progeny can be sterile, and maintained in a sterile environment. Such TCPC (and their progeny) and cultures thereof can also be included in a medium, such as a liquid medium suitable for administration to a subject (e.g., a mammal such as a human).

Methods for producing genetically modified TCPC expressing at least one of TCF-1, TCF-3, TCF-4 or TCF-10 (a.k.a. LEF-1), and their differentiated progeny are provided herein. In one embodiment, the method includes obtaining a tissue or blood sample, isolating one or more cells from the sample, selecting one or more cells based upon morphology or phenotypic marker expression profile, thereby isolating an TCPC.

Methods for producing TCPC and TCPC populations are also provided, including expanding TCPC for a desired number of cell divisions, thereby producing increased numbers or a population of TCPC. Relative proportions or amounts of TCPC within cell cultures include 50%, 60%, 70%, 80%, 90% or more TCPC in a population of cells.

Methods for producing a differentiated progeny cell of a genetically modified TCPC expressing at least one of TCF-1, TCF-3, TCF-4 or TCF-10 (e.g., a progenitor cell, a precursor cell, a developmental intermediate, a differentiated T cell) are also provided.

In one embodiment, the invention includes a T cell derived from a genetically modified TCPC expressing at least one of TCF-1, TCF-3, TCF-4 or TCF-10 (a.k.a. LEF-1). In one embodiment, the invention includes a method of making a T cell derived from genetically modified TCPC expressing at least one of TCF-1, TCF-3, TCF-4 or TCF-10 (a.k.a. LEF-1).

The quality of the T cells derived from the genetically modified TCPCs may be detected morphologically, by the presence of a T cell differentiation related cell surface marker, or by expression of cell differentiation-related transcript detectable by RT-PCR. Other agents may be added to this culture system for the proliferation and viability of the T cells, such as serum (e.g., fetal bovine or human serum), interleukin-2 (IL-2), insulin, IFN-γ, IL-4, IL-7, GM-CSF, IL-10, IL-12, IL-15, TGFβ, SCF, Flt3-L and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells.

The ability of the T cells derived from genetically modified TCPC expressing at least one of TCF-1, TCF-3, TCF-4 or TCF-10 to function in vivo may be studied using animal models or in clinical trials.

Genetically modified TCPC expressing at least one of TCF-1, TCF-3, TCF-4 or TCF-10 and their progeny can be used for various applications in accordance with the methods of the invention including treatment and therapeutic methods. The invention therefore provides in vivo and ex vivo treatment and therapeutic methods that employ genetically modified TCPC, populations of genetically modified TCPC, and progeny of genetically modified TCF-1.

Genetically modified TCPC expressing at least one of TCF-1, TCF-3, TCF-4 or TCF-10 (a.k.a. LEF-1), and their progeny, can be can be administered to a subject, or used as a cell-based therapy, or to provide secreted factors, to provide a benefit to a subject (e.g., by differentiating into T cells in the subject, or to stimulate, increase, induce, promote, enhance or augment activity or function of the endogenous immune system in the subject).

### Therapy

The invention contemplates the use of the cells of the invention in in vitro, in vivo, and ex vivo settings. Thus, the invention provides for use of the cells of the invention for research purposes and for therapeutic or medical/veterinary purposes. In research settings, an enormous number of practical applications exist for the technology.

In accordance with the invention, methods of providing a cellular therapy and methods of treating a subject having a disease or disorder that would benefit from a cellular therapy are provided. In one embodiment, the method includes administering at least one progeny cell (e.g., a T cell) derived from a genetically modified TCPC expressing at least one of TCF-1, TCF-3, TCF-4 or TCF-10 to a subject in an amount sufficient to provide a benefit to the subject. In various embodiments, the subject having a disease or disorder involving T cell deficiency, such as T cell deficiency following bone marrow ablation, T cell deficiency following bone marrow transplant, T cell deficiency following chemotherapy, and T cell deficiency following corticosteroid therapy.

In one embodiment, the invention includes a method of treating a subject having a disease or disorder, including the step of administering to the subject at least one T cell derived from at least one genetically modified TCPC, wherein the genetically modified TCPC comprises a nucleic acid encoding at least one of TCF-1, TCF-3, TCF-4 or TCF-10 (a.k.a. LEF-1). In some embodiments, the nucleic acid encoding TCF-1 comprises the nucleic acid sequence of SEQ ID NO:37, or a modification thereof. In some embodiments, the nucleic acid encoding TCF-3 comprises the nucleic acid sequence of SEQ ID NO:39, or a modification thereof. In some embodiments, the nucleic acid encoding TCF-4 comprises the nucleic acid sequence of SEQ ID NO:41, or a modification thereof. In some embodiments, the nucleic acid encoding TCF-10 comprises the nucleic acid sequence of SEQ ID NO:43, or a modification thereof. In various embodiments, the TCPC useful in the method of deriving a T cell includes at least one of an ESC, an iPSC, a HSC, a HPC, a CLP, an ELP, an ETP, an LMPP and a lineage marker-negative cell, such as an LSK. In some embodiments, the TCPC is stably transfected with nucleic acid encoding TCF-1, while in other embodiments the TCPC is transiently transfected. In various embodiments, the T cell derived by the methods of the invention expresses at least one of CD2, CD3, CD25, CD4 and CD8.

Genetically modified TCPC expressing at least one of TCF-1, TCF-3, TCF-4 or TCF-10 (a.k.a. LEF-1), or their progeny, can be administered or delivered to a subject by any route suitable for the treatment method or protocol. Specific non-limiting examples of administration and delivery routes include parenteral, e.g., intravenous, intramuscular, intrathecal (intra-spinal), intrarterial, intradermal, intrathymic, subcutaneous, intra-pleural, transdermal (topical), transmucosal, intra-cranial, intra-ocular, mucosal, implantation and transplantation.

In some embodiments, the genetically modified TCPC expressing at least one of TCF-1, TCF-3, TCF-4 or TCF-10 (a.k.a. LEF-1), or their progeny, is autologous with respect to the subject; that is, the TCPC used in the method were obtained or derived from a cell obtained from the subject that is treated according to the method. In other embodiments, the genetically modified TCPC or the progeny of the genetically modified TCPC is allogeneic with respect to the subject; that is, the TCPC used in the method were obtained or derived from a cell obtained from a subject that is different than the subject that is treated according to the method.

The methods of the invention also include administering genetically modified TCPC expressing at least one of TCF-1, TCF-3, TCF-4 or TCF-10 (a.k.a. LEF-1), or progeny of genetically modified TCPC, prior to, concurrently with, or following administration of additional pharmaceutical agents or biologics. Pharmaceutical agents or biologics may activate or stimulate the genetically modified TCPC or their progeny. Non-limiting examples of such agents include, for example, interleukin-2 (IL-2), insulin, IFN-γ, IL-4, IL-7, GM-CSF, IL-10, IL-12, IL-15, TGFβ, SCF, Flt3-L and TNF-α.

The methods of the invention also include methods that provide a detectable or measurable improvement in a condition of a given subject, such as alleviating or ameliorating one or more signs or symptoms of a disease or disorder, such as, for example, a disease or disorder involving T cell deficiency.

In the methods of treatment of the invention, the method can be practiced one or more times (e.g., 1-10, 1-5 or 1-3 times) per day, week, month, or year. The skilled artisan will know when it is appropriate to delay or discontinue administration. Frequency of administration is guided by clinical need or surrogate markers. Of course, as is typical for any treatment or therapy, different subjects will exhibit different responses to treatment and some may not respond or respond less than desired to a particular treatment protocol, regimen or process. Amounts effective or sufficient will therefore depend at least in part upon the disorder treated (e.g., the type or severity of the disease, disorder, illness, or pathology), the therapeutic effect desired, as well as the individual subject (e.g., the bioavailability within the subject, gender, age, etc.) and the subject's response to the treatment based upon genetic and epigenetic variability (e.g., pharmacogenomics).

The present invention also pertains to kits useful in the methods of the invention. Such kits comprise various combinations of components useful in any of the methods described elsewhere herein, including for example, hybridization probes or primers (e.g., labeled probes or primers), antibodies, reagents for detection of labeled molecules, materials for the amplification of nucleic acids, medium, media supplements, components for deriving a T cell derived from a genetically modified TCPC expressing at least one of TCF-1, TCF-3, TCF-4 or TCF-10 (a.k.a. LEF-1), a genetically modified TCPC cell expressing at least one of TCF-1, TCF-3, TCF-4 or TCF-10 (a.k.a. LEF-1), and instructional material. For example, in one embodiment, the kit comprises components useful for deriving a T cell from a genetically modified TCPC.

A label or packaging insert can include appropriate written instructions, for example, practicing a method of the invention. Thus, in additional embodiments, a kit includes a label or packaging insert including instructions for practicing a method of the invention in solution, in vitro, in vivo, or ex vivo. Instructions can therefore include instructions for practicing any of the methods of the invention described herein. Instructions may further include indications of a satisfactory clinical endpoint or any adverse symptoms or complications that may occur, storage information, expiration date, or any information required by regulatory agencies such as the Food and Drug Administration for use in a human subject.

Genetically modified TCPC or their progeny can be included in or employ pharmaceutical formulations. Pharmaceutical formulations include “pharmaceutically acceptable” and “physiologically acceptable” carriers, diluents or excipients. The terms “pharmaceutically acceptable” and “physiologically acceptable” mean that the formulation is compatible with pharmaceutical administration. Such pharmaceutical formulations are useful for, among other things, administration or delivery to, implantation or transplant into, a subject in vivo or ex vivo.

As used herein the term “pharmaceutically acceptable” and “physiologically acceptable” mean a biologically acceptable formulation, gaseous, liquid or solid, or mixture thereof, which is suitable for one or more routes of administration, in vivo delivery or contact. Such formulations include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical formulations can be made to be compatible with a particular local, regional or systemic administration or delivery route. Thus, pharmaceutical formulations include carriers, diluents, or excipients suitable for administration by particular routes. Specific non-limiting examples of routes of administration for compositions of the invention are parenteral, e.g., intravenous, intramuscular, intrathecal (intra-spinal), intrarterial, intradermal, intrathymic, subcutaneous, intra-pleural, transdermal (topical), transmucosal, intra-cranial, intra-ocular, mucosal administration, and any other formulation suitable for the treatment method or administration protocol.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

Supplementary compounds (e.g., preservatives, antioxidants, antimicrobial agents including biocides and biostats such as antibacterial, antiviral and antifungal agents) can also be incorporated into the compositions. Pharmaceutical compositions may therefore include preservatives, anti-oxidants and antimicrobial agents.

Preservatives can be used to inhibit microbial growth or increase stability of ingredients thereby prolonging the shelf life of the pharmaceutical formulation. Suitable preservatives are known in the art and include, for example, EDTA, EGTA, benzalkonium chloride or benzoic acid or benzoates, such as sodium benzoate. Antioxidants include, for example, ascorbic acid, vitamin A, vitamin E, tocopherols, and similar vitamins or provitamins.

Pharmaceutical formulations and delivery systems appropriate for the compositions and methods of the invention are known in the art (see, e.g., Remington: The Science and Practice of Pharmacy (2003) 20th ed., Mack Publishing Co., Easton, Pa.; Remington's Pharmaceutical Sciences (1990) 18th ed., Mack Publishing Co., Easton, Pa.; The Merck Index (1996) 12th ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11th ed., Lippincott Williams & Wilkins, Baltimore, Md.; and Poznansky et al., Drug Delivery Systems (1980), R. L. Juliano, ed., Oxford, N.Y., pp. 253-315).

Activation and Expansion of T Cells Derived from Genetically Modified TCPC

T cells derived from genetically modified TCPC can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention are expanded by contact with a surface having attached thereto an agent that stimulates a CD3/TCR complex associated signal and a ligand that stimulates a co-stimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4′ T cells or CD8′ T cells, an anti-CD3 antibody and an anti-CD28 antibody. Examples of an anti-CD28 antibody include 9.3, B-T3, XR-CD28 (Diaclone, Besançon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In certain embodiments, the primary stimulatory signal and the co-stimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one embodiment, the agent providing the co-stimulatory signal is bound to a cell surface and the agent providing the primary activation signal is in solution or coupled to a surface. In certain embodiments, both agents can be in solution. In another embodiment, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one embodiment, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the co-stimulatory signal is an anti-CD28 antibody or antigen-binding fragment thereof; and both agents are co-immobilized to the same bead in equivalent molecular amounts. In one embodiment, a 1:1 ratio of each antibody bound to the beads for CD4′ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular embodiment an increase of from about 1 to about 3 fold is observed as compared to the expansion observed using a ratio of 1:1. In one embodiment, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain embodiments of the invention, the ratio of anti CD28 antibody to anti CD3 antibody bound to the beads is greater than 2:1. In one particular embodiment, a 1:100 CD3:CD28 ratio of antibody bound to beads is used. In another embodiment, a 1:75 CD3:CD28 ratio of antibody bound to beads is used. In a further embodiment, a 1:50 CD3:CD28 ratio of antibody bound to beads is used. In another embodiment, a 1:30 CD3:CD28 ratio of antibody bound to beads is used. In one preferred embodiment, a 1:10 CD3:CD28 ratio of antibody bound to beads is used. In another embodiment, a 1:3 CD3:CD28 ratio of antibody bound to the beads is used. In yet another embodiment, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain embodiments the ratio of cells to particles ranges from 1:100 to 100:1 and any integer values in-between and in further embodiments the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In one embodiment, a ratio of particles to cells of 1:1 or less is used. In one particular embodiment, a preferred particle:cell ratio is 1:5. In further embodiments, the ratio of particles to cells can be varied depending on the day of stimulation. For example, in one embodiment, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular embodiment, the ratio of particles to cells is 1:1 on the first day of stimulation and adjusted to 1:5 on the third and fifth days of stimulation. In another embodiment, particles are added on a daily or every other day basis to a final ratio of 1:1 on the first day, and 1:5 on the third and fifth days of stimulation. In another embodiment, the ratio of particles to cells is 2:1 on the first day of stimulation and adjusted to 1:10 on the third and fifth days of stimulation. In another embodiment, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type.

In further embodiments of the present invention, the cells, such as T cells, are combined with agent-coated beads, the beads and the cells are subsequently separated, and then the cells are cultured. In an alternative embodiment, prior to culture, the agent-coated beads and cells are not separated but are cultured together. In a further embodiment, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached to contact the T cells. In one embodiment the cells (for example, 10⁴ to 10⁹ T cells) and beads (for example, DYNABEADS® M-450 CD3/CD28 T paramagnetic beads at a ratio of 1:1) are combined in a buffer, preferably PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain embodiments, it may be desirable to significantly decrease the volume in which particles and cells are mixed together (i.e., increase the concentration of cells), to ensure maximum contact of cells and particles. For example, in one embodiment, a concentration of about 2 billion cells/ml is used. In another embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain embodiments. For example, using high concentration of cells allows more efficient selection of CD8+ T cells that normally have weaker CD28 expression.

In one embodiment of the present invention, the mixture may be cultured for several hours (about 3 hours) to about 14 days or any hourly integer value in between. In another embodiment, the mixture may be cultured for 21 days. In one embodiment of the invention the beads and the T cells are cultured together for about eight days. In another embodiment, the beads and T cells are cultured together for 2-3 days. Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-12, IL-15, TGFβ, SCF, Flt3-L and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

T cells that have been exposed to varied stimulation times may exhibit different characteristics. In addition to CD4 and CD8 markers, other phenotypic markers vary significantly, but in large part, reproducibly during the course of the cell expansion process. Thus, such reproducibility enables the ability to tailor a T cell product for specific purposes.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### A Critical Role for TCF-1 in T-Lineage Specification and Differentiation

The vertebrate thymus provides an inductive environment for T-cell development. Within the mouse thymus, Notch signals are indispensable for imposing the T-cell fate on multipotential hematopoietic progenitors, but the downstream effectors that impart T-lineage specification and commitment are not well understood. It is demonstrated herein that a transcription factor, T-cell factor 1 (TCF-1; also known as transcription factor 7, T-cell specific, TCF7), is a critical regulator in T-cell specification. TCF-1 is highly expressed in the earliest thymic progenitors, and its expression is upregulated by Notch signals. Most importantly, when TCF-1 is forcibly expressed in bone marrow (BM) progenitors, it drives the development of T-lineage cells in the absence of T-inductive Notch1 signals. Further characterization of these TCF-1-induced cells revealed expression of many T-lineage genes, including T-cell-specific transcription factors Gata3 and Bcl11b, and components of the T-cell receptor. The data presented herein suggest a model where Notch signals induce TCF-1, and TCF-1 in turn imprints the T-cell fate by upregulating expression of T-cell essential genes.

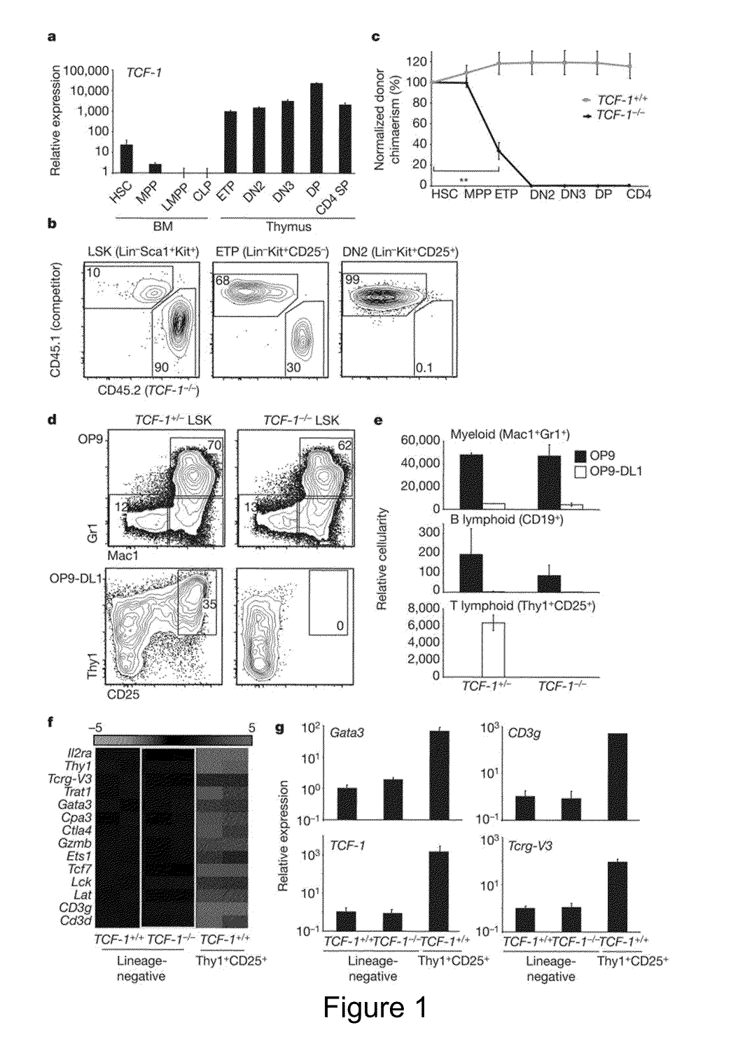

Within the thymus, Notch1 signals are known to drive development through sequential steps during which alternative lineage potentials are lost and T-lineage-specific gene expression (specification) occurs, but Notch's downstream effectors have thus far remained unclear. It is described herein that the high mobility group (HMG) box transcription factor, TCF-1, is highly upregulated in early thymic progenitors (ETPs; FIG. 1A) and that TCF-1 expression is upregulated when progenitors are exposed to Notch1 signals.

The materials and methods employed in these experiments are now described.

Mice

Mice were males or females, age 5-18 weeks. C57BL/6 (CD45.2) and B6-Ly5.2 (CD45.1) mice were purchased from the NCI animal facility. Other mice used were Tcf7^{-/-} (TCF-1^{-/-} ΔVII) mice (Verbeek et al., 1995, Nature, 374: 70-74), Notch1^{f/f}MxCre⁺Rosa^{YFP/+} mice (Liu et al., 2011, Clin. Invest., 121: 800-808), and β-catenin^{f/f}MxCre^{+/-} mice (Brault et al., 2001, Development, 128: 1253-1264).

Intravenous Transfers and Intrathymic Injections

Chimeric mice were generated by intravenously injecting T-cell-depleted TCF-1^{+/+} or TCF-1^{-/-} BM (CD45.2) that was mixed with wild-type T-depleted BM (CD45.1) at 1:1 or 2:1 ratios into lethally-irradiated (900 rad) mice. Mice were analyzed after 12-14 weeks for donor chimerism. Notch1^{f/f}MxCre^{+Rosa}^{YFP/+} LSK progenitors were transduced with TCF-1 or control virus; 24 hours later 2×10⁴ cells were intrathymically injected into sublethally (650 rad) irradiated mice (CD45.1). Mice were analyzed 10-16 days later. For intrathymic injections of TCF-1-expressing Thy1⁺CD25⁺ cells, cells were isolated by cell sorting from day 8 cultures and 3×10⁵ cells were injected into sublethally irradiated mice and analyzed for thymic reconstitution 1-3 weeks later.

OP9 and OP9-DL Cell Culture

OP9-GFP (OP9), OP9-DL1, and OP9-DL4 cells were provided and used as described (Schmitt et al., 2006, Immunol. Rev., 209: 95-102).

Administration of Poly(I:C)

β-catenin^{f/f}MxCre^{+/-} mice were induced as described previously (Huang et al., 2009, J. Clin. Invest, 119: 3519-3529). Poly(I:C) (Sigma-Aldrich) was resuspended in Dulbecco PBS at 2 mg ml⁻¹. Mice received intraperitoneal injections of 0.2 mg poly(I:C) every other day for 2 weeks. Notch1^{f/f}MxCre⁺Rosa^{YFP/+} mice received two intraperitoneal injections of 0.2 mg poly(I:C) 1 week apart and were rested for 1 week.

Intravenous Transfers and Intrathymic Injections

For intravenous transfers of transduced progenitors, wild-type LSK progenitors were transduced with TCF-1, ICN1 or control virus and transferred into sublethally irradiated mice. Mice were analyzed 2-8 weeks after reconstitution for donor chimerism in BM, spleen and thymus.

For intrathymic injection of TCF-1^{-/-} or TCF-1^{+/+} progenitors, fresh LSK progenitors were isolated by cell sorting and injected intrathymically. Mice were analyzed after 10 days for thymic reconstitution.

Plasmids

MSCV-IRES-GFP (MIGR1) and MIGR1-ICN1 retroviral vectors were obtained from W. Pear. MSCV-VEX (VEX) vector was provided by C. Klug. MigR1 and VEX vectors were converted to Gateway®-compatible vectors (Invitrogen) and full-length TCF-1 cDNA was cloned into VEX according to the Gateway® clonase manual (Invitrogen). The mouse TCF-1 promoter (˜1.5 kb insert containing TCF-1 promoter activity based on Promoter Prediction 2.0; Knudsen, 1999, Bioinformatics, 15: 356-361) was cloned into pGL3 basic promoter vector. A ˜1.3 kb insert containing the −31 kb CSL binding site of TCF-1 (in relation to the full-length TCF-1 translational start site) was cloned into pGL3 promoter vector (Promega). Mutation of the TCF-1 binding site in pGL3 basic-mouse TCF-1 promoter or the −31 kb CSL binding site in the pGL3 promoter vector was achieved with site-directed mutagenesis.

Sequences

An example TCF-1 nucleotide sequence is:

(SEQ ID NO: 37)
cccgccagctcgcggagccgctctgccccgcgccctagcccgcgcctgcagcccgcccaggcggagt
cagcccgcgctccgcccgccgcgatccgagctcggaggttcggactccgggctcgccgccccccgggccggctccgcgcc
ccgcactcccggcgcccagcgccccgcgccccggcgggcggagcgcaccatgccgcagctggactccggcgggggcgg
cgcgggcggcggcgacgacctcggcgcgccggacgagctgctggccttccaggatgaaggcgaggagcaggacgacaag
agccgcgacagcgccgccggtcccgagcgcgacctggccgagctcaagtcgtcgctcgtgaacgagtccgagggcgcggc
cggcggcgcagggatcccgggggtcccgggggccggcgccggggcccgcggcgaggccgaggctctcgggcgggaac
acgctgcgcagagactcttcccggacaaacttccagagcccctggaggacggcctgaaggccccggagtgcaccagcggca
tgtacaaagagaccgtctactccgccttcaatctgctcatgcattacccacccccctcgggagcagggcagcacccccagccgc
agcccccgctgcacaaggccaatcagcccccccacggtgtcccccaactctctctctacgaacatttcaacagcccacatccca
cccctgcacctgcggacatcagccagaagcaagttcacaggcctctgcagacccctgacctctctggcttctactccctgacctc
aggcagcatggggcagctcccccacactgtgagctggttcacccacccatccttgatgctaggttctggtgtacctggtcaccca
gcagccatcccccacccggccattgtgcccccctcagggaagcaggagctgcagcccttcgaccgcaacctgaagacacaa
gcagagtccaaggcagagaaggaggccaagaagccaaccatcaagaagcccctcaatgccttcatgctgtacatgaaggaga
tgagagccaaggtcattgcagagtgcacacttaaggagagcgctgccatcaaccagatcctgggccgcaggtggcacgcgct
gtcgcgagaagagcaggccaagtactatgagctggcccgcaaggagaggcagctgcacatgcagctatacccaggctggtc
agcgcgggacaactacgggaagaagaagaggcggtcgagggaaaagcaccaagaatccaccacaggaggaaaaagaaat
gcattcggtacttacccggagaaggccgctgccccagccccgttccttccgatgacagtgctctaggctgccccgggtccccag
ctccccaggactcaccctcataccatctgctgccccgcttccccacagaactgcttactagccctgcggagccggcacctacatc
cccaggtctctccactgctctcagcctcccaaccccagggcccccacaggccccccgcagcaccctgcagagcacacaggta
cagcaacaggaatctcagagacaggtggcctagcaggcacaggacacctggccgcctccaggagcctaccccctgaaagtg
acagagacccagatctcatggaaactggccaggggtcctgttaacgtcatctcagggtccagaccctgaagatttcagaggctg
cagaacttctgcctgaacctggggtcatcgattcaaactgctccaagtggtgggaatcagatctgtcttgatgtgtcatctaattaag
ggaatcccttgtacctatggctgcctgcatctattctttgtaccatctgtcttgccagccagaagcctctgcctccctagcttttctgct
ataggtcagagatgggctgaactgagcctagctaccttctctacccatctcccccatcccccactgccacaccctccccattcaga
cacttcatggaccaagaatgagctggtttgtcaaacaacatgtgagcatggtcacaagcacaaagctcaagatgacagctcttct
aaggaaatggagaagctctgtttataaaaacaaaaacaaaaccagctgctactcataagttggaccagaggaagccccttactat
gatctcaggagcttgcaagaagcaggaaggggaatggaataggttaagtttaggcctatcaacctaagcaacagaaataatctg
acactaccttatcaggcaaattggggaggggagggtgtatctagctctagttcaaattatttgaaagtgttccctgagaaacccacc
agcctaagaagctctggccccaggcttgtcactagcagctgcagtcaacagttcaaagaagtcatggcccaaatccagtgtgca
cccctccccattcacagagcctttttcacaattccatttccagttcatctatggcagtccagccagctcctgggcagcttgagaggg
caaacccaaaacctcatgacagccagagcctgtctttcagcattcagtccgcctggccggctccagtttccccatggggctgcg
ggacagaggaccattacaactagatcaaggagcccagaaaacctccagtagtggacaacaggttttcaccatagcctacgttaa
cccatttttgagccaagcttcaaccctcagccttgaaaaacaagtctttaatttaatttttgttttttgcctaaatccaaagaaaaaggg
ctgtcgggccaggcgcggtggctcacgcctgtaatcccagcactttggcaggccgaggcaggtggatcacctgacgtcagtag
tttgagaccagcctggccaacatggtgaaaccctgtatctactaaaaatacaaaaattagccggacgtggtggtgcgcgcatgta
atcccagctactcgggaggctgaggcggaagaatcccttgaacccgggaggcggaggttccagtgagccgaggtggcgctat
tgcactccagtctgggtaacagggagactgcatctcaaaaaaaaaaaaaaaaaaaaaaaaaa

An example TCF-1 amino acid sequence is:

(SEQ ID NO: 38)
MPQLDSGGGGAGGGDDLGAPDELLAFQDEGEEQDDKSRDSAAGPERDLA
ELKSSLVNESEGAAGGAGIPGVPGAGAGARGEAEALGREHAAQRLFPDK
LPEPLEDGLKAPECTSGMYKETVYSAFNLLMHYPPPSGAGQHPQPQPPL
HKANQPPHGVPQLSLYEHFNSPHPTPAPADISQKQVHRPLQTPDLSGFY
SLTSGSMGQLPHTVSWFTHPSLMLGSGVPGHPAAIPHPAIVPPSGKQEL
QPFDRNLKTQAESKAEKEAKKPTIKKPLNAFMLYMKEMRAKVIAECTLK
ESAAINQILGRRWHALSREEQAKYYELARKERQLHMQLYPGWSARDNYG
KKKRRSREKHQESTTGGKRNAFGTYPEKAAAPAPFLPMTVL

An example TCF-3 nucleotide sequence is:

(SEQ ID NO: 39)
ggtttccaggcctgaggtgcccgccctggccccaggagaatgaaccagccgcagaggatggcgcctgtgggcacagacaag
gagctcagtgacctcctggacttcagcatgatgttcccgctgcctgtcaccaacgggaagggccggcccgcctccctggccgg
ggcgcagttcggaggttcaggtcttgaggaccggcccagctcaggctcctggggcagcggcgaccagagcagctcctccttt
gaccccagccggaccttcagcgagggcacccacttcactgagtcgcacagcagcctctcttcatccacattcctgggaccggg
actcggaggcaagagcggtgagcggggcgcctatgcctccttcgggagagacgcaggcgtgggcggcctgactcaggctg
gcttcctgtcaggcgagctggccctcaacagccccgggcccctgtccccttcgggcatgaaggggacctcccagtactacccc
tcctactccggcagctcccggcggagagcggcagacggcagcctagacacgcagcccaagaaggtccggaaggtcccgcc
gggtcttccatcctcggtgtacccacccagctcaggtgaggactacggcagggatgccaccgcctacccgtccgccaagaccc
ccagcagcacctatcccgcccccttctacgtggcagatggcagcctgcacccctcagccgagctctggagtcccccgggcca
ggcgggcttcgggcccatgctgggtgggggctcatccccgctgcccctcccgcccggtagcggcccggtgggcagcagtgg
aagcagcagcacgtttggtggcctgcaccagcacgagcgtatgggctaccagctgcatggagcagaggtgaacggtgggctc
ccatctgcatcctccttctcctcagcccccggagccacgtacggcggcgtctccagccacacgccgcctgtcagcggggccga
cagcctcctgggctcccgagggaccacagctggcagctccggggatgccctcggcaaagcactggcctcgatctactccccg
gatcactcaagcaataacttctcgtccagcccttctacccccgtgggctccccccagggcctggcaggaacgtcacagtggcct
cgagcaggagcccccggtgccttatcgcccagctacgacgggggtctccacggcctgcagagtaagatagaagaccacctg
gacgaggccatccacgtgctccgcagccacgccgtgggcacagccggcgacatgcacacgctgctgcctggccacggggc
gctggcctcaggtttcaccggccccatgtcactgggcgggcggcacgcaggcctggttggaggcagccaccccgaggacgg
cctcgcaggcagcaccagcctcatgcacaaccacgcggccctccccagccagccaggcaccctccctgacctgtctcggcct
cccgactcctacagtgggctagggcgagcaggtgccacggcggccgccagcgagatcaagcgggaggagaaggaggacg
aggagaacacgtcagcggctgaccactcggaggaggagaagaaggagctgaaggccccccgggcccggaccagcagtac
ggacgaggtgctgtccctggaggagaaagacctgagggaccgggagaggcgcatggccaataacgcgcgggagcgggtg
cgcgtgcgggatattaacgaggccttccgggagctggggcgcatgtgccagatgcacctcaagtcggacaaagcgcagacca
agctgctcatcctgcagcaggccgtgcaggtcatcctggggctggagcagcaggtgcgagagcggaacctgaatcccaaagc
agcctgtttgaaacggcgagaagaggaaaaggtgtcaggtgtggttggagacccccagatggtgctttcagctccccacccag
gcctgagcgaagcccacaaccccgccgggcacatgtgaaagtaaacaaaacctgaaagcaagcaacaaaacatacactttgt
cagagaagaaaaaaatgccttaactataaaaagcggagaaatggaaacatatcactcaagggggatgctgtggaaacctggctt
attcttctaaagccaccagcaaattgtgcctaagcgaaatattttttttaaggaaaataaaaacattagttacaagattttttttttcttaat
gtagatgaaaattagcaaggatgctgcctttggtctctggtttttttaagctttttttgcatatgttttgtaagcaacaaatttttttgtataa
aagtcccgtgtctctcgctatttctgctgctgttcctagactgagcattgcatttcttgatcaaccagatgattaaacgttgtattaaaaa
gaccccgtgtaaacctgagcccccccgtccccccccccccccggaagccactgcacacagacagaacggggacaggcggc
gggtcttttgtttttttgatgttgggggttctcttggttttgtcatgtggaaagtgatgcgtgggcgttccctgatgaaggcaccttggg
gcttccctgccgcatcctctcccctcaggaaggggactgacctgggcttgggggaagggacgtcagcaaggtggctctgaccc
tcccaggtgactctgccaagcagctgtggcccccagggctaccctacacaacgccctccccaggcccccctaagctgctctcc
cttggaacctgcacagctctctgaaatggggcattttgttgggaccagtgacccctggcatggggaccacaccctggagcccgg
tgctggggacctcctggacaccctgtccttcactcctttgccccagggacccaggctcatgctctgaactctggctgagaggatg
ctgctcaggagccagcacaggacaccccccaccccaccccaccatgtccccattacaccagagggccatcgtgacgtagaca
ggatgccaggggcctggccagcctcccccaatgctggggagcatccctgggcctggggccacacctgctgccctccctctgt
gtggtccaagggcaagagtggctggagccgggggactgtgctggtctgagccccacgaaggccttgggctgtgcgtccgacc
ctgctgcagaaccagcagggtgtcccctcgggcccatctgtgtcccatgtcccagcacccaggcctctctccaggtctccttttct
ggtcttttgccatgagggtaaccagctcttcccagctggctggggactgtcttgggtttaaaactgcaagtctcctaccctgggatc
ccatccagttccacacgaactagggcagtggtcactgtggcacccaggtgtgggcctggctagctgggggccttcatgtgccct
tcatgcccctccctgcattgaggccttgtggacccctgggctggctgtgttcatccccgctgcaggtcgggcgtctccccccgtg
ccactcctgagactcccaccgttacccccaggagatcctggactgcctgactcccctccccagactggcttgggagcctgggcc
ccatggtagatgcaagggaaacctcaaggccagctcaatgcctggtatctgcccccagtccaggccaggcggaggggaggg
gctgtccggctgcctctcccttctcggtggcttcccctacgccctgggagtttgatctcttaagggaacttgcctctccctcttgttttg
ctcctggccctgcccctaggtctgggtgggcagtggccccatagcctctggaactgtgcgttctgcatagaattcaaacgagatt
cacccagcgcgaggaggaagaaacagcagttcctgggaaccacaattatggggggtggggggtgtgatctgagtgcctcaag
atggttttcaaaaaaatttttttaaagaaaataattgtatacgtgtcaacacagctggctggatgattgggactttaaaacgaccctctt
tcaggtggattcagagacctgtcctgtatataacagcactgtagcaataaacgtgacattttataacgatgc

An example TCF-3 amino acid sequence is:

(SEQ ID NO: 40)
MNQPQRMAPVGTDKELSDLLDFSMMFPLPVTNGKGRPASLAGAQFGGSG
LEDRPSSGSWGSGDQSSSSFDPSRTFSEGTHFTESHSSLSSSTFLGPGL
GGKSGERGAYASFGRDAGVGGLTQAGFLSGELALNSPGPLSPSGMKGTS
QYYPSYSGSSRRRAADGSLDTQPKKVRKVPPGLPSSVYPPSSGEDYGRD
ATAYPSAKTPSSTYPAPFYVADGSLHPSAELWSPPGQAGFGPMLGGGSS
PLPLPPGSGPVGSSGSSSTFGGLHQHERMGYQLHGAEVNGGLPSASSFS
SAPGATYGGVSSHTPPVSGADSLLGSRGTTAGSSGDALGKALASIYSPD
HSSNNFSSSPSTPVGSPQGLAGTSQWPRAGAPGALSPSYDGGLHGLQSK
IEDHLDEAIHVLRSHAVGTAGDMHTLLPGHGALASGFTGPMSLGGRHAG
LVGGSHPEDGLAGSTSLMHNHAALPSQPGTLPDLSRPPDSYSGLGRAGA
TAAASEIKREEKEDEENTSAADHSEEEKKELKAPRARTSSTDEVLSLEE
KDLRDRERRMANNARERVRVRDINEAFRELGRMCQMHLKSDKAQTKLLI
LQQAVQVILGLEQQVRERNLNPKAACLKRREEEKVSGVVGDPQMVLSAP
HPGLSEAHNPAGHM

An example TCF-4 nucleotide sequence is:

(SEQ ID NO: 41)
gtgtgtggatgtgtgagtgagagggaacgagagtaagagaaagaaagaagtgaggggatgtaaactcgaataaatttcaaagt
gcctccgagggatgcaacgggcaaaaactgaactgttcaggcttcagattgtaactgacgatctgaggaaaaatgaggtgctcg
atgaattttcgtttgtattttttggcgaggcgggggaggtgttgagattttttttttttcccctcggggtgggtgcgagggggatgcatc
ctagcctgcccgacccggagcaagtcgcgtctccccgccggagcccccccacccatttctttgctgaacttgcaattccgtgcgc
ctcggcgtgtttccccctccccccttccctccgtcccctcccctccccggagaagagagttggtgttaagagtcagggatcttggc
tgtgtgtctgcggatctgtagtggcggcggcggcggcggcggcggggaggcagcaggcgcgggagcgggcgcaggagca
ggcggcggcggtggcggcggcggttagacatgaacgccgcctcggcgccggcggtgcacggagagccccttctcgcgcgc
gggcggtttgtgtgattttgctaaaatgcatcaccaacagcgaatggctgccttagggacggacaaagagctgagtgatttactg
gatttcagtgcgatgttttcacctcctgtgagcagtgggaaaaatggaccaacttctttggcaagtggacattttactggctcaaatg
tagaagacagaagtagctcagggtcctgggggaatggaggacatccaagcccgtccaggaactatggagatgggactcccta
tgaccacatgaccagcagggaccttgggtcacatgacaatctctctccaccttttgtcaattccagaatacaaagtaaaacagaaa
ggggctcatactcatcttatgggagagaatcaaacttacagggttgccaccagcagagtctccttggaggtgacatggatatggg
caacccaggaaccctttcgcccaccaaacctggttcccagtactatcagtattctagcaataatccccgaaggaggcctcttcaca
gtagtgccatggaggtacagacaaagaaagttcgaaaagttcctccaggtttgccatcttcagtctatgctccatcagcaagcact
gccgactacaatagggactcgccaggctatccttcctccaaaccagcaaccagcactttccctagctccttcttcatgcaagatgg
ccatcacagcagtgacccttggagctcctccagtgggatgaatcagcctggctatgcaggaatgttgggcaactcttctcatattc
cacagtccagcagctactgtagcctgcatccacatgaacgtttgagctatccatcacactcctcagcagacatcaattccagtcttc
ctccgatgtccactttccatcgtagtggtacaaaccattacagcacctcttcctgtacgcctcctgccaacgggacagacagtataa
tggcaaatagaggaagcggggcagccggcagctcccagactggagatgctctggggaaagcacttgcttcgatctattctcca
gatcacactaacaacagcttttcatcaaacccttcaactcctgttggctctcctccatctctctcagcaggcacagctgtttggtctag
aaatggaggacaggcctcatcgtctcctaattatgaaggaccatacactctttgcaaagccgaattgaagatcgtttagaaagact
ggatgatgctattcatgttctccggaaccatgcagtgggcccatccacagctatgcctggtggtcatggggacatgcatggaatc
attggaccttctcataatggagccatgggtggtctgggctcagggtatggaaccggccttctttcagccaacagacattcactcat
ggtggggacccatcgtgaagatggcgtggccctgagaggcagccattctcttctgccaaaccaggttccggttccacagcttcct
gtccagtctgcgacttcccctgacctgaacccaccccaggacccttacagaggcatgccaccaggactacaggggcagagtgt
ctcctctggcagctctgagatcaaatccgatgacgagggtgatgagaacctgcaagacacgaaatcttcggaggacaagaaatt
agatgacgacaagaaggatatcaaatcaattactaggtcaagatctagcaataatgacgatgaggacctgacaccagagcaga
aggcagagcgtgagaaggagcggaggatggccaacaatgcccgagagcgtctgcgggtccgtgacatcaacgaggctttca
aagagctcggccgcatggtgcagctccacctcaagagtgacaagccccagaccaagctcctgatcctccaccaggcggtggc
cgtcatcctcagtctggagcagcaagtccgagaaaggaatctgaatccgaaagctgcgtgtctgaaaagaagggaggaagag
aaggtgtcctcagagcctccccctctctccttggccggcccacaccctggaatgggagacgcatcgaatcacatgggacagat
gtaaaagggtccaagttgccacattgcttcattaaaacaagagaccacttccttaacagctgtattatcttaaacccacataaacact
tctccttaacccccatttttgtaatataagacaagtctgagtagttatgaatcgcagacgcaagaggtttcagcattcccaattatcaa
aaaacagaaaaacaaaaaaaagaaagaaaaaagtgcaacttgagggacgactttctttaacatatcattcagaatgtgcaaagc
agtatgtacaggctgagacacagcccagagactgaacggcaatctttccacactgtggaacaatgcatttgtgcctaaacttctttt
ggaaaaaaaaaatataattaatttgtaagtctgaaaaaaaaatatttaatttaaaaaaaattgtaaacttgcaataatgaaaaagtgta
cttctgaagaaaactacatgaacgtttttgttggtattcaagtcagctagtgtttataattactggatattgaattaggggaagctcggc
tgccctagtaacaaaaccagcaaacgtcctgatgacaacgaagtgatgacattagccattccttagggtaggaggaacagatgg
atcttatagacctatgacaaatatatatataaatatatatataaatatatattaaaaatttagtgactatggtaagcttttgttcatttgtttc
agacttttttctcctgtaaaaaaatagtactgattaacttttttaaaagaaagattttactgtaaatatggatttttttttttttggtcttatttct
gtccctttccctggtttgttatcgtaacctgtagtgccaactctgcttccagaggggtagtgcaggatgaaatgctgaccctgatgtt
gcttctcattcataaataagtagaaagttgtttctccagtcttttgggaacacaggacttaaaagtcacatcatgtgtagatattacaa
gcagcattaccaagacatggcaaaaagagtttgtctgaattgtaatgttgcgtttgtgaacctattctgggattttcagaggtacaag
gttagaatgctacaatgttaccactgtgccttccaatgtttatatcatcggaaacataacataatcaaagtggctgtgatttaacaaaa
tgattaaagtgttacctacctgtgtagccgaagtagtgtgcagtgaggcgtttctgaatacatggtcagatttttggaaaaaaacaaa
aacaaaaaaaacaagtaaagttcaaaaaccgtcaaatgagaaaattgcaagtagtgtgacagagctgattgattttgttgctttctt
gattttttttttcaaaatgggtttactaaaatgtagatgacttaactgcctcctccttcgtctgaaaaatgccaatattcaatcatcatgca
gcattataacaagccttataagtcctaaagcattaagttgcacttttttgaggaggggtagtgcagtatttctctggccagtatgaatg
aagtttatacttaccatatttgatagaaacatagatcaagctatggcacagcgactcatcagatagctagctttgacgtctgggcac
aattgaaccaacttccatcgtgaatctttataatgattgactttggtgtatagtgcagtaaacaaatagtgctcctagttaagtatttgtc
agcatccttttgtctctaacttgtttctatttttacagccacacaattcttggcatgtattaagaaaaaaaaaaatccctgttcaagtagtt
tttccacctatcagcactgagtaaatgccataaatccattgaaatggtctaaatgttccatctgttctcctgttttgccagttatatagta
atgaaatacatttgtaaattttatgcaacaaatggcaaacgtatcattattttgaaattgtgtatgtaaaagttatatttttacatgtagact
cttgttattatgtgttttaatacattgtatcagtttttgtttttttttaaaaactgtggtttaaaaagaagtctcatttaaatgaaatagctaca
agaatcagaattttatgttcatttctgaaaatgtaagaacaaataagatagttaccacgtggtcatcttttacaaacccataaacatttt
gattagctgtgtgtgtgttgaaaaactgtaaatatgttcagtagcgataaaactaaaataactttgatttgttgataagttcctaaaatgt
ggaggtggattaaaaccttaggagaatagcagaaatcaaacttcatgaaaagttattttggggctttcctgtgaaatgtatgaacaa
agaggctcagagaaggacatggaagacaataatgtatactctctcctcctccctgaataatgaaaaccatgtgtatttgttccctcc
gtatgttaaagatttccttttagtggtacattctgcactcattttgtatagtctaccaaggcgggtatccctaggaacaatattatatagg
aagcaggtatactctgatcacattcaggataagtgtacagaagaaaatacggtgtttactctttagggaactggaaacactccctg
cattgatgtacattttaagaatggcacttttgatacatgttatcataaaggtgcttaatagagctgaattaaagtttttcaaatctgtaaa
caaagcaaaaaagtaaattgtagtcatttgattattttttaaattggtgattatattttgttctcactcagagtaaaagctgcaatttattg
ttcaccagctttgatgtattcattactcagtaatgcaatacctctattgttgaattccctttggaaataagtgaaaattctaacggccact
gaaagctgctcgctaggttttgcttggtggagaaacataatctgcacctatccatattaattgggttgtatccccattaaaaaagaaa
aaaagggaatgtggcctttttagtgtgttttttattgttgttgttttgtaattatcaaacccaggtaagatattggtatcctgcactggattt
tcaaatgaagttcagcagaagacagttaagattaaagtactatacaaaaatttcaaaagggtccatactacgctatctgtatgacga
cacttaggctggggatctctttcagaaactcggactttaaaagcaacttggagcagttgatccacctccacattcaagtaatttatga
atatgcagaatagggatctgttcatctagaaatttttaccatttgtcttctgtgtagctgcaaggaacactaatgtttatacaactgtca
gtccacccagtggtgcaactggttctgattcagtcttccgattcctttttatttttcactttttcctatttctgaatttttttttttatttgtgatct
tgattttgatgaggggttggggagtggggagggagtcgaaccaagacttggagttaagaggattttcatcttttgcatccaacagg
cagaatatgatctgtgtccaaaagtgaacttgagtcaggaatgaatcaatttcagcataaacaagcacaaaaatttagtctgctggc
tgactggaagcaaaaaagtcaagatggaatatgatgaattccaacacaatggggcaccaaggcctttaggcctctctttttattttg
ctttggttttgtttgtttttctttagagacatgctctttctcatgggacttgaagtggactcatctttgtgcagtgctggttttgccatactca
tttcaagtattatagacatatgtaatggtgaaaatatatgaactgtggcctttttcattcttgttacttgtgatgcaattaagtgaagataa
gaaaaaaaaaaaaaaagcagagatttaccatgtatcagtgcctggctttttgttataaagctttgtttgtctagtgctcttttgctataaa
atagactgtagtacaccctagtaggaaaaaaaaaaaactaaatttaaaaataaaaaatatatttggcttatttttcgcaggagcaatc
cttttataccatgaatattacaaaaaaattgtcagattctgaatatttcttctttgtagatttttggaatcattatgagtaaaagtttgttactt
tattttactatttaaaagatgttattttaccatgtgttaccaagatgaaactgtatgggtagcttttttgtttgttttttgttttgtttttgtttttgt
ttttgtttttagttgtaggtcgcagcggggaaattttttgcgactgtacacatagctgcagcattaaaaacttaaaaaaattgttaaaaa
aaaaaaaagggaaaacatttcaaaaaaaaaaaaaaagataaacagttacaccttgttttcaatgtgtggctgagtgcctcgatttttt
catgtttttggtgtatttctgatttgtagaagtgtccaaacaggttgtgtgctggagttccttcaagacaaaaacaaacccagcttggt
caaggccattacctgtttcccatctgtagttattcgatgaagtcatgtacatgaccgttctgtagcaataaatgtgccatttttataaact
gtttctgacacttgtttcatttcattttgcattgtccatatagctatgattctcttctgtaagtaaaacgcatctatatttcattttccaagtgtt
ggaggtattgacagcttaacaaacaaaacatacaaaaaaaatcacaaaaacaaattgaaaagcaaagcacatgattgatcaagg
aagagatgcccttaatgaaaatggaacgggatgcatgcaaaacaaaaagaaaactgtctagaggattaactaattgaaggaatat
aattaatgtgtgtgtaacactgaagctatgcatttgaagagctctgaactgcaccagtgttttcggttgtgctgcaggttgctaagtca
agtcagccttaaccttttgcaccagttggtcggctgtttggcagaacattctcagatcttttcagtcaaaaatctaagatgatttattttg
tatcactttgttaaaagctgaatattgttaactacagttaatattaacactgtatttatactttctcaaactacatccgccccaccacttct
ggttgcctctgttgactattaatccagatgtaaacaaccagatgtttttttctaacttgtacaaactgacgtgtgtcaactatcatggaa
ggaaaaaaatgtacagattaaaattattcagtgttatgtactgtaagttaatatttttgtagaatggacatcaatctactttgcaaaattt
ggaggctatttcaacattgcactgtagaaatgtaaagtaatgtatgcaatgtaaaggaaagcccgcggtagctgagcgcttcataa
cagaatgttctaatcaagtacgtggtatttggggatgtctccaatattgctcttgtattctttctaattgggtttagtgactagttgaagg
aaaatgttataacgccatttggttcacatgtgaagtgccctccatagccaaatgttgggatttttttttttttcgtttttggttggactgttt
gcagatatttaaattttatgaaatttccaaagattttggttgataacccccttttaccttctaaatgatttgagatgttcttatgttcttactgt
gtgttttaaatatatataaaagagccacaagcattt

An example TCF-4 amino acid sequence is:

(SEQ ID NO: 42)
MHHQQRMAALGTDKELSDLLDFSAMFSPPVSSGKNGPTSLASGHFTGSN
VEDRSSSGSWGNGGHPSPSRNYGDGTPYDHMTSRDLGSHDNLSPPFVNS
RIQSKTERGSYSSYGRESNLQGCHQQSLLGGDMDMGNPGTLSPTKPGSQ
YYQYSSNNPRRRPLHSSAMEVQTKKVRKVPPGLPSSVYAPSASTADYNR
DSPGYPSSKPATSTFPSSFFMQDGHHSSDPWSSSSGMNQPGYAGMLGNS
SHIPQSSSYCSLHPHERLSYPSHSSADINSSLPPMSTFHRSGTNHYSTS
SCTPPANGTDSIMANRGSGAAGSSQTGDALGKALASIYSPDHTNNSFSS
NPSTPVGSPPSLSAGTAVWSRNGGQASSSPNYEGPLHSLQSRIEDRLER
LDDAIHVLRNHAVGPSTAMPGGHGDMHGIIGPSHNGAMGGLGSGYGTGL
LSANRHSLMVGTHREDGVALRGSHSLLPNQVPVPQLPVQSATSPDLNPP
QDPYRGMPPGLQGQSVSSGSSEIKSDDEGDENLQDTKSSEDKKLDDDKK
DIKSITRSRSSNNDDEDLTPEQKAEREKERRMANNARERLRVRDINEAF
KELGRMVQLHLKSDKPQTKLLILHQAVAVILSLEQQVRERNLNPKAACL
KRREEEKVSSEPPPLSLAGPHPGMGDASNHMGQM

An example TCF-10 nucleotide sequence is:

(SEQ ID NO: 43)
agcgccggcgaggcgcgggaggaggagaagcagtggggaggcgcagccgctcacctgcggggcagggcgcggaggag
ggacccgggctgcgcgctctcgggccgaggaaccaggacgcgcccggagcctcgcacgcggccaagctcggggcgtccc
ctcccctcggccgggcgaactcaaggggcgcagctctttgctttgacagagctggccggcggaggcgtgcagagcggcgag
ccggcgagccaggctgagaaactcgagccgggaacaaagaggggtcggactgagtgtgtgtgtcggctcgagctccgggca
gaggcatttgggcccgaggcccccgctgtgactccccgagactccgcagtgccctccactgcggagtccccgcgcttgccgg
caaaaactttattcttggcaaacttctctttctcttcccctcctcctcggcccccatcttctgctcctcctccttctctagcagattaaatg
agcctcgagaagaaaaaccgaagcgaaagggaagaaaataagaagatctaaaacggacatctccagcgtgggtggctcctttt
tctttttctttttttcccacccttcaggaagtggacgtttcgttatcttctgatccttgcaccttcttttggggcaaacggggcccttctgc
ccagatcccctctcttttctcggaaaacaaactactaagtcggcatccggggtaactacagtggagagggtttccgcggagacgc
gccgccggaccctcctctgcactttggggaggcgtgctccctccagaaccggcgttctccgcgcgcaaatcccggcgacgcg
gggtcgcggggtggccgccggggcagcctcgtctagcgcgcgccgcgcagacgcccccggagtcgccagctaccgcagc
cctcgccgcccagtgcccttcggcctcgggggcgggcgcctgcgtcggtctccgcgaagcgggaaagcgcggcggccgcc
gggattcgggcgccgcggcagctgctccggctgccggccggcggccccgcgctcgcccgccccgcttccgcccgctgtcct
gctgcacgaacccttccaactctcctttcctcccccacccttgagttacccctctgtctttcctgctgttgcgcgggtgctcccacag
cggagcggagattacagagccgccgggatgccccaactctccggaggaggtggcggcggcgggggggacccggaactct
gcgccacggacgagatgatccccttcaaggacgagggcgatcctcagaaggaaaagatcttcgccgagatcagtcatcccga
agaggaaggcgatttagctgacatcaagtcttccttggtgaacgagtctgaaatcatcccggccagcaacggacacgaggtgg
ccagacaagcacaaacctctcaggagccctaccacgacaaggccagagaacaccccgatgacggaaagcatccagatgga
ggcctctacaacaagggaccctcctactcgagttattccgggtacataatgatgccaaatatgaataacgacccatacatgtcaaa
tggatctctttctccacccatcccgagaacatcaaataaagtgcccgtggtgcagccatcccatgcggtccatcctctcacccccc
tcatcacttacagtgacgagcacttttctccaggatcacacccgtcacacatcccatcagatgtcaactccaaacaaggcatgtcc
agacatcctccagctcctgatatccctactttttatcccttgtctccgggtggtgttggacagatcaccccacctcttggctggttttcc
catcatatgattcccggtcctcctggtccccacacaactggcatccctcatccagctattgtaacacctcaggtcaaacaggaaca
tccccacactgacagtgacctaatgcacgtgaagcctcagcatgaacagagaaaggagcaggagccaaaaagacctcacatt
aagaagcctctgaatgatttatgttatacatgaaagaaatgagagcgaatgtcgttgctgagtgtactctaaaagaaagtgcagct
atcaaccagattcttggcagaaggtggcatgccctctcccgtgaagagcaggctaaatattatgaattagcacggaaagaaaga
cagctacatatgcagctttatccaggctggtctgcaagagacaattatggtaagaaaaagaagaggaagagagagaaactaca
ggaatctgcatcaggtacaggtccaagaatgacagctgcctacatctgaaacatggtggaaaacgaagctcattcccaacgtgc
aaagccaaggcagcgaccccaggacctcttctggagatggaagcttgttgaaaacccagactgtctccacggcctgcccagtc
gaccccaaaggaacactgacatcaattttaccctgaggtcactgctagagacgctgatccataaagacaatcactgccaacccct
ctttcgtctactgcaagagccaagttccaaaataaagcataaaaaggttttttaaaaggaaatgtaaaagcacatgagaatgctag
caggctgtggggcagctgagcagcttttctcccctcatatctgcgtgcacttcccagagcatcttgcatccaaacctgtaacctttc
ggcaaggacggtaacttggctgcatttgcctgtcatgcgcaactggagccagcaaccagcacatccatcagcaccccagtgga
ggagttcatggaagagttccctctttgtttctgcttcatttttctttcttttcttttctcctaaagcttttatttaacagtgcaaaaggatcgttt
ttttttgcttttttaaacttgaatttttttaatttacactttttagttttaattttcttgtatattttgctagctatgagcttttaaataaaattgaaag
ttctggaaaagtttgaaataatgacataaaaagaagccttctttttctgagacagcttgtctggtaagtggcttctctgtgaattgcctg
taacacatagtggcttctccgcccttgtaaggtgttcagtagagctaaataaatgtaatagccaaacccactctgttggtagcaattg
gcagccctatttcagtttattttttcttctgttttcttcttttctttttttaaacagtaaaccttaacagatgcgttcagcagactggtttgcag
tgaattttcatttctttccttatcacccccttgttgtaaaaagcccagcacttgaattgttattactttaaatgttctgtatttgtatctgttttt
attagccaattagtgggattttatgccagttgttaaaatgagcattgatgtacccattttttaaaaaagcaaggcacagcctttgccca
aaactgtcatcctaacgtttgtcattccagtttgagttaatgtgctgagcatttttttaaaagaagctttgtaataaaacatttttaaaaatt
gtcatttaaaaaaaaaaaaaaaaaa

An example TCF-10 amino acid sequence is:

(SEQ ID NO: 44)
MPQLSGGGGGGGGDPELCATDEMIPFKDEGDPQKEKIFAEISHPEEEGD
LADIKSSLVNESEIIPASNGHEVARQAQTSQEPYHDKAREHPDDGKHPD
GGLYNKGPSYSSYSGYIMMPNMNNDPYMSNGSLSPPIPRTSNKVPVVQP
SHAVHPLTPLITYSDEHFSPGSHPSHIPSDVNSKQGMSRHPPAPDIPTF
YPLSPGGVGQITPPLGWFSHHMIPGPPGPHTTGIPHPAIVTPQVKQEHP
HTDSDLMHVKPQHEQRKEQEPKRPHIKKPLNAFMLYMKEMRANVVAECT
LKESAAINQILGRRWHALSREEQAKYYELARKERQLHMQLYPGWSARDN
YGKKKKRKREKLQESASGTGPRMTAAYI

Cell Preparations, Flow Cytometry and Cell Sorting

BM and thymocytes were prepared as previously described (Schwarz et al., 2007, J. Immunol., 178: 2008-2017). Cell preparations were stained with optimized antibody dilutions. Antibodies used in the lineage cocktail (Lin) include antibodies against B220 (RA3-6B2), CD19 (1D3), CD11b/Mac1 (M1/70), Gr1 (8C5), CD11c (HL3), NK1.1 (PK136), TER119 (TER-119), CD3ε (2C11), CD8a (53-6.7), CD813 (53-5.8), TCRβ (H57), γδTCR (GL-3). Additional antibodies used included antibodies against CD45^{B6} (104), CD45^{SJL} (A20), Sca1 (E13-161.7), Kit (2B8), Flt3 (A2F10.1), CD90.1/Thy1.1 (HIS51), Gr1 (RB6-8C5), CD19 (ID3) and CD25 (PC61.5). Antibodies were directly conjugated to fluorescein isothiocyanate (FITC), phycoerythrin (PE), PE-Cy5, PE-Cy5.5, peridinin-chlorophyll-protein complex (PerCP)-Cy5.5, PE-Cy7, allophycocyanin (APC), APC-Cy5.5 (or Alexa 700), APC-Cy7 (or APCeFluor780), or biotin. Biotinylated antibodies were revealed with Streptavidin PE-Texas Red. All antibodies were purchased from eBiosciences, Biolegend, or BD Pharmingen. Cell sorting was performed on a FACSAria II (BD Biosciences) and flow cytometric analysis was performed on a LSR-II (BD Biosciences). Dead cells were excluded through 4,6 diamidino-2-phenylindole (DAPI) uptake. Doublets were excluded through forward scatter-height by forward scatter-width and side scatter-height by side scatter-width parameters. Data were analyzed using FlowJo (Tree Star). The LSK population was isolated as Lin⁻Sca1⁺Kit⁺. HSCs were sorted as Lin⁻Sca1⁺Kit⁺Flt3⁻CD150⁺ BM cells; LMPPs (the ‘lymphoid primed’ subset of MPPs) sorted as Lin⁻Sca1⁺Kit⁺Flt3^{hi} BM cells. Thymocyte populations were defined and cell-sorted as ETP (Lin^{-/lo} Kit⁻ CD25⁻), DN2 (Lin^{-/lo} Kit⁺ CD25⁺), DN3 (Lin^{-/lo} Kit CD25⁺). Total thymocytes were stained and sorted as immature ISP (CD4⁻ CD8⁺ TCRβ⁻), DP (CD4⁺ CD8⁺), CD4 SP (CD4⁺ CD8⁻), and CD8 SP (CD8⁺ CD4⁻ TCRβ⁺).

Retroviral Transduction

Retroviral packaging was performed as previously described (Pui et al., 1999, Immunity, 11: 299-308), with the exceptions of packaging cell line 293T cells and transfection reagent FuGENE 6 (Roche) in place of CaPO₄. Hematopoietic progenitors were transduced using RetroNectin (Takara). Briefly, 24 or 12-well plates were coated with 20-100 μg ml⁻¹ RetroNectin according to the manufacturer's instructions. High-titre retroviral supernatants were added into wells, centrifuged at 25° C. for 1-2 hours, following which viral supernatant was removed. Cell-sorted progenitor cells were resuspended in the stimulation cocktails including DMEM-complete medium, 1% penicillin/streptomycin, 15% fetal calf serum (FCS), 1-glutamate (2 mM), IL-3 (10 ng ml⁻¹), IL-6 (10 ng ml⁻¹), SCF (20 ng ml⁻¹), Flt3-ligand (20 ng ml⁻¹), Polybrene (4 μg ml⁻¹) and added to virus-bound RetroNectin-coated plates. Transduced BM progenitors were sorted 36-48 hours post-infection.

Luciferase Gene Reporter Assay

For luciferase reporter assays, 293T cells were seeded 1 day before transfection to reach 80% confluency. 293T cells were transiently cotransfected with FuGENE 6 (Roche) following instructions according to manufacturer's protocol. Constructs used include: pGL3 vector (300 ng per well) containing the TCF-1 promoter with a TCF-1 binding site or a mutated TCF-1 binding site, the pGL3 promoter vector containing the wild-type −31 kb CSL binding site in TCF-1 locus or a mutated version, the TOPFLASH TCF-1 reporter, and with either empty vector MigR1, MigR1-ICAT, MigR1-TCF-1, or MigR1-ICN1 (300 ng per well). Renilla was added at 50 ng per well to control for transfection efficiency. DMEM containing 10% 1-glutamine, 10% penicillin/streptomycin was added 24 hours post transfection and cells were harvested 40-48 hours after transfection and analyzed with a Dual Assay Reporter Kit (Promega). Data were analyzed by comparing luciferase activity to Renilla activity and adjusted to the fold increase over background.

Quantitative RT-PCR

RNA was purified from indicated cell types with the RNeasy MicroKit (Qiagen) and reverse transcribed to cDNA, using SuperScript II Kit (Invitrogen). Real-time PCR was performed with PCR Master Mix, using TaqMan probes specific for indicated genes (Applied Biosystems), and analyzed on ABI Prism 7900 system (Applied Biosystems). Relative transcript abundance was determined by using the ΔΔCₜ or ΔCₜ method after normalization with 18S, or GAPDH. All samples were run in triplicate. Error bars represent s.e.m.

ChIP

ChIP was performed with the ChIP assay kit (Millipore), all procedures have been described (Yashiro-Ohtani et al., 2009, Genes Dev., 23: 1665-1676). In brief, CD4/CD8-depleted (DN) thymocytes or Scid-adh cells were fixed and immunoprecipitated with IgG control antibody (rabbit IgG; Santa Cruz Biotechnologies), Notch1 TAD/PEST-specific antiserum (Weng et al., 2006, Genes Dev., 20: 2096-2109), or anti-TCF-1 (C63D9) (Cell Signaling). DNA was purified using a PCR purification kit (Qiagen) and eluted by water. QRT-PCR was performed using the SYBR Green primers that flank putative TCF-1 or CSL binding sites. All genomic distances greater than 2 kb away from the translational start site were rounded to the nearest kilobase. All distances are relative to the translational start site. Primer sequences are listed in Table 1. The relative DNA amount was calculated using the standard curve method. The input DNA was defined as an aliquot of sheared chromatin before immunoprecipitation, and was used to normalize the sample to the amount of chromatin added to each ChIP. All results are the average of triplicate PCR amplifications and results were confirmed for reproducibility in separate experiments.

Gene Expression Analysis

All protocols were conducted as described in the Affymetrix GeneChip Expression Analysis Technical Manual. RNA was extracted from sorted cells, and the quality and quantity of the RNA was tested on a bioanalyzer. This was followed by the Affymetrix WT Terminal Labelling kit for fragmentation and biotinylation according to the manufacturers' instructions. Biotinylated targets were heated at 99° C. for 5 minutes and hybridized for 16 hours at 45° C. The microarrays were then washed at low (6×SSPE) and high (100 mM MES, 0.1 M NaCl) stringency and stained with streptavidin-phycoerythrin. Fluorescence was amplified by adding biotinylated anti-streptavidin and an additional aliquot of streptavidin-phycoerythrin stain. GeneChips were scanned using the GeneArray Scanner 3000 7G. The data were analyzed using Partek Genomics Suite, version 6.5 (Partek). Robust multichip average (RMA) with default settings was used to normalize data. Gene signal values for the arrays were log₂-transformed and heat maps represent the log₂-transformed normalized signals values or fold-change values compared to a reference population. Heat maps were generated using Matrix2png, a publicly available software (Pavlidis et al., 2003, Bioinformatics, 19: 295-296).

Statistical Analysis

The means of each data set were analyzed using Student's t-test, with a two-tailed distribution assuming equal sample variance.

The results of the experiments are now described.

### TCF-1 in Normal T-Lymphopoiesis

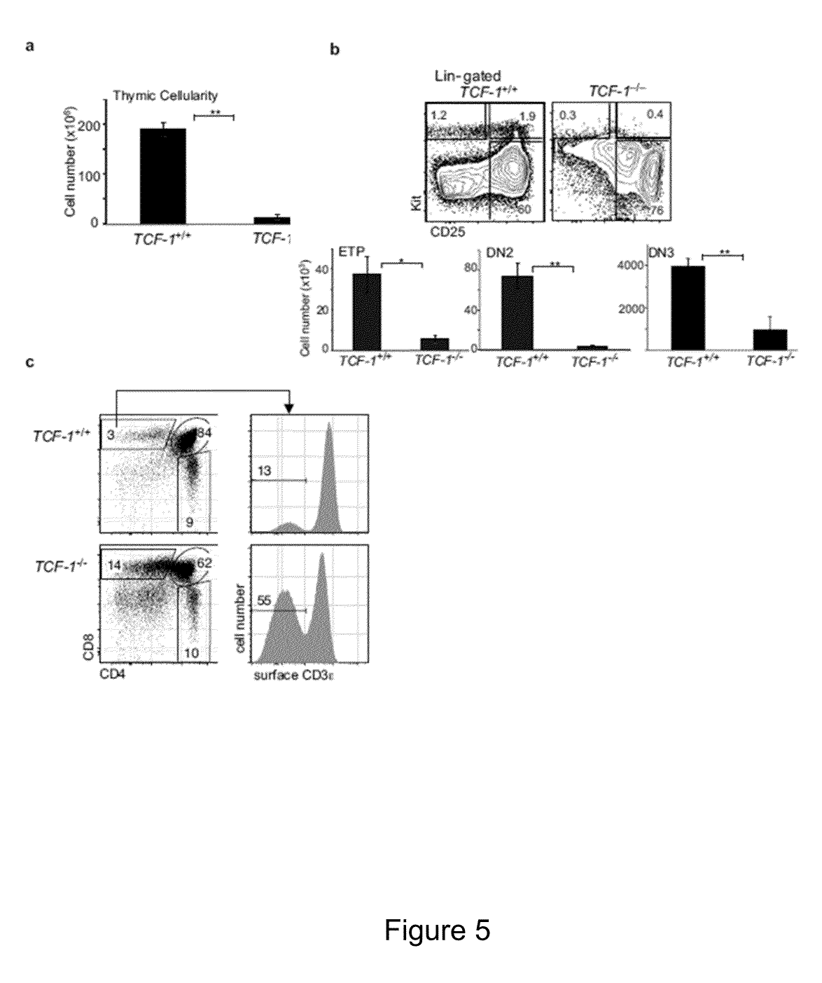

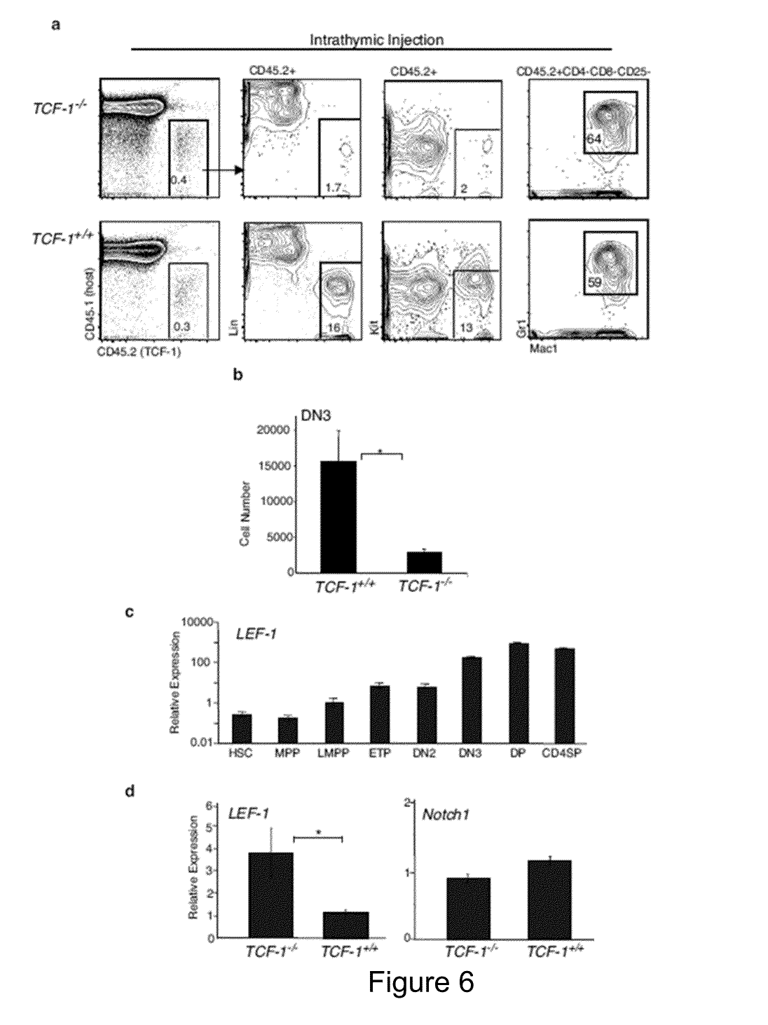

TCF-1 deficiency greatly reduces thymic cellularity but does not abrogate T-cell development (Verbeek et al., 1995, Nature, 374: 70-74; Schilham et al., 1998, J. Immunol., 161: 3984-3991; Goux et al., 2005, Blood, 106: 1726-1733) (FIG. 5). When TCF-1^{-/-} progenitors were assessed in the absence of competition in irradiated mice, small numbers of T-lineage cells developed (FIG. 6A). The related transcription factor LEF-1 can compensate for TCF-1 (Okamura et al., 1998, Immunity, 8: 11-20); consistently, TCF-1^{-/-} DN3 cells showed elevated LEF-1 expression (FIG. 6D). To examine more rigorously the requirement for TCF-1 in early progenitors, TCF-1 progenitors were placed in competition with wild-type cells in mixed BM chimaeras. TCF-1^{-/-} progenitors reconstituted BM progenitor populations but were defective in generating ETPs, and downstream thymic populations were almost entirely absent (FIG. 1B and FIG. 1C). These data indicated a marked requirement for TCF-1 at very early stages of T-cell development, which was clearly revealed when TCF-1-deficient progenitors were placed in competition with TCF-1-sufficient cells.

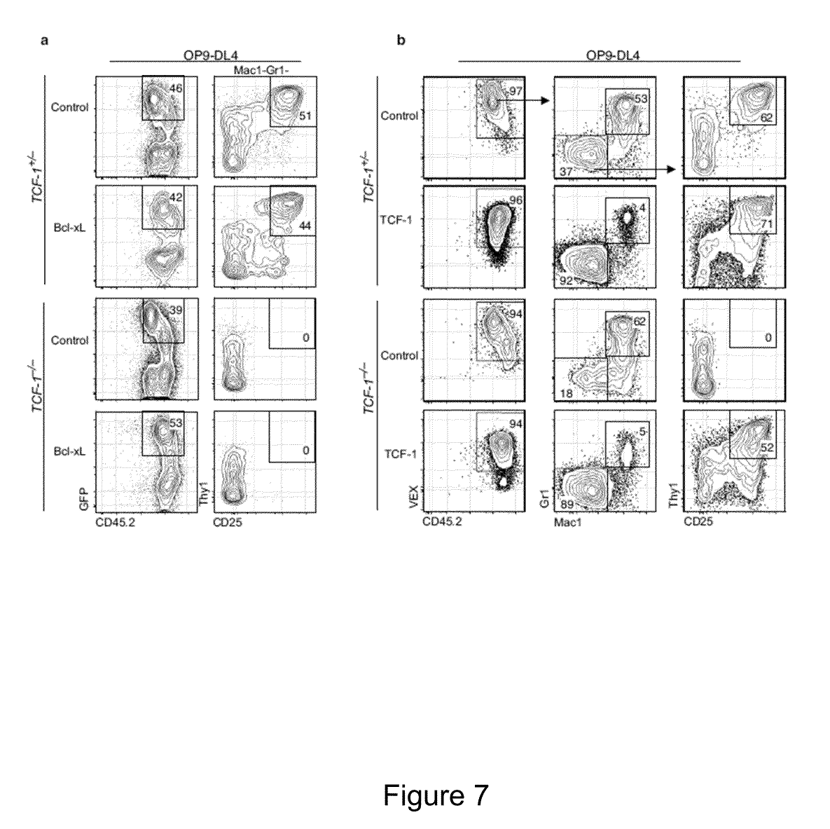

To elucidate more precisely the role of TCF-1 in early T-cell development, stromal cells expressing Notch ligands (OP9-DL4 or OP9-DL1) were used. In this system, hematopoietic progenitors that respond to Notch signals differentiate into immature Thy1⁺CD25⁺ T-lineage cells (Schmitt et al., 2006, Immunol. Rev., 209: 95-102; Huang et al., 2005, J. Immunol., 175: 4858-4865). Both TCF-1^{+/-} and TCF-1^{-/-} Lin⁻Sca1⁺Kit⁺ (LSK) progenitors generated myeloid and B-lineage cells on control OP9 stroma and these fates were appropriately inhibited when progenitors were signaled through Notch. On OP9-DL1 stroma, however, TCF-1^{-/-} progenitors failed to give rise to T-lineage cells (FIG. 1D and FIG. 1E), even when the survival factor Bcl-xL was ectopically expressed (FIG. 7A). Hence TCF-1 is dispensable for initial Notch1-mediated inhibition of alternative fates but is involved in promoting the T-cell fate.

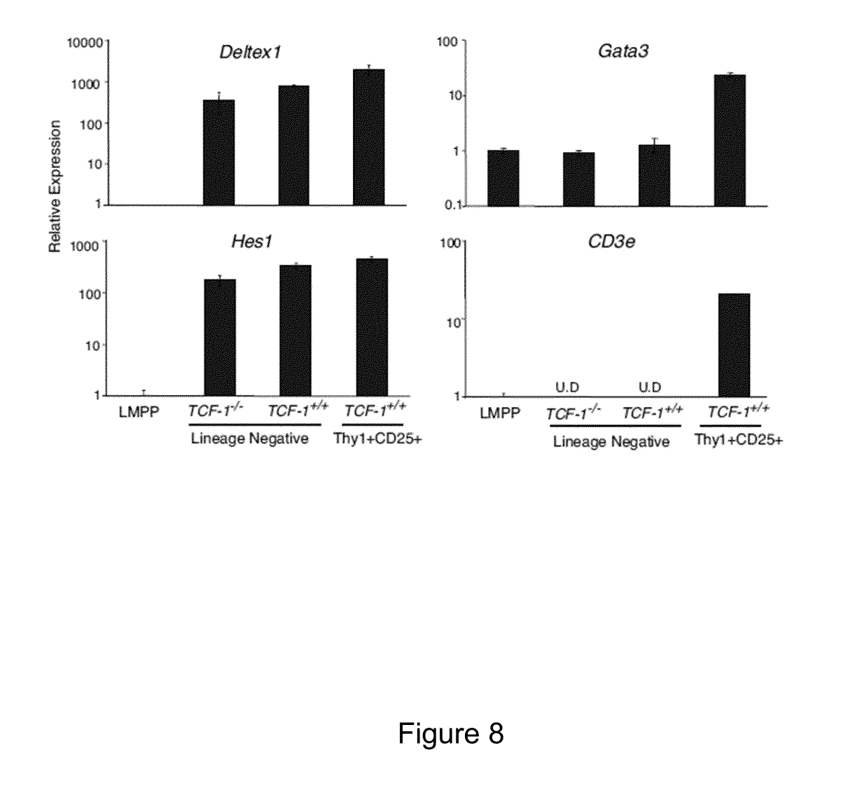

To better examine the requirement for TCF-1 in promoting T-cell development, TCF-1^{-/-} and TCF-1^{+/+} lymphoid-primed multipotent progenitors (LMPPs) were cultured on OP9-DL4 for 4 days and performed global gene expression analysis on TCF-1^{-/-} and TCF-1^{+/+} lineage-negative precursors as well as TCF-1^{+/+} Thy1⁺CD25⁺ T-lineage cells. It was found that TCF-1^{-/-} progenitors failed to upregulate expression of many T-lineage genes (FIG. 1F and FIG. 1G). Both TCF-1^{+/+} and TCF-1^{-/-} progenitors upregulated expression of Notch target genes Deltex1 (also known as Dtx1) and Hes1 (FIG. 8), confirming that TCF-1-deficient progenitors sense Notch signals, but cannot upregulate expression of T-cell genes.

### TCF-1 Drives Early T-Cell Development

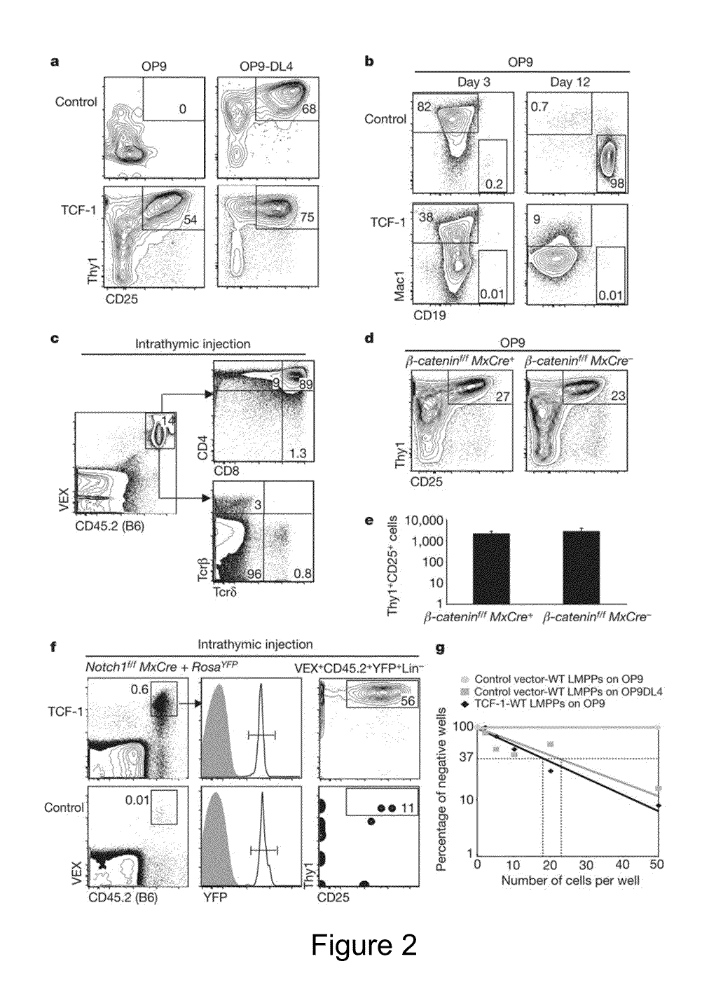

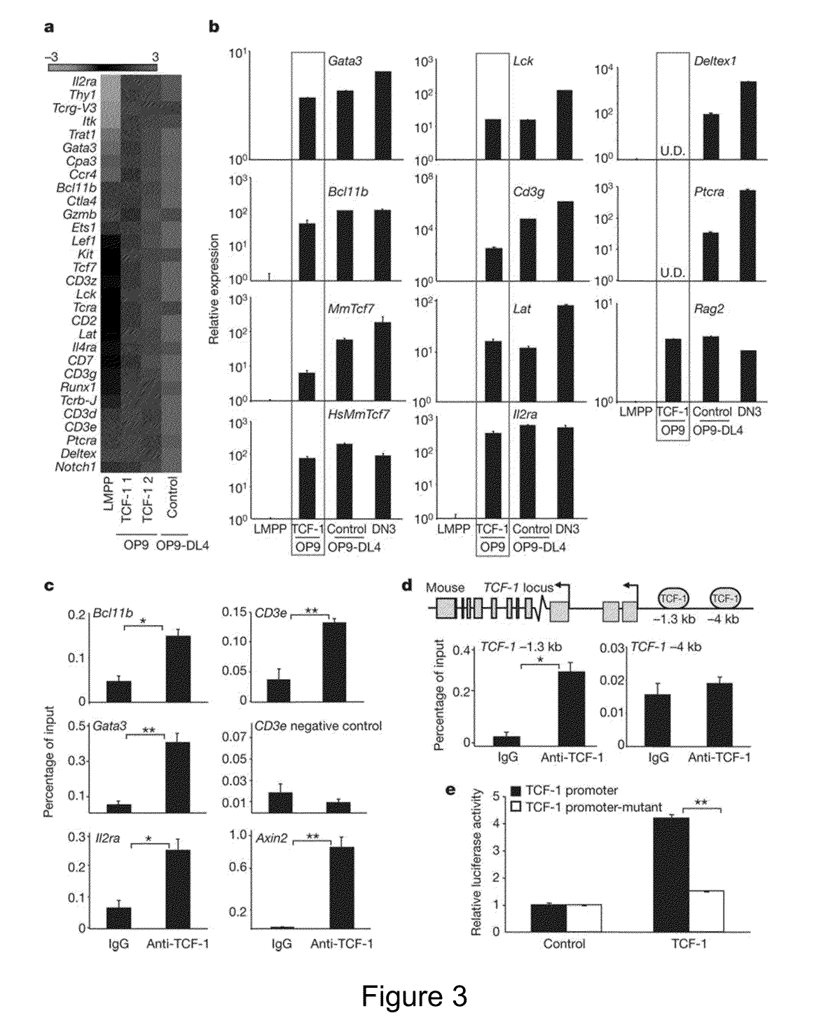

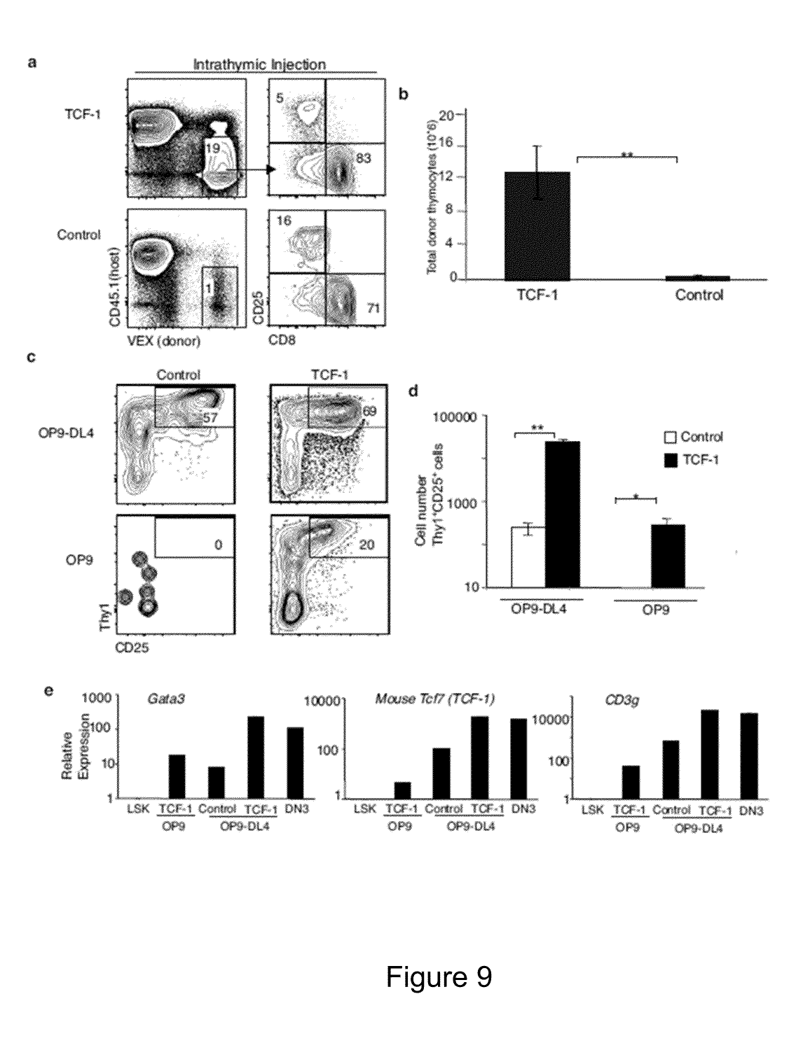

To investigate the possibility that TCF-1 initiates T-lineage gene expression, human TCF-1 was expressed ectopically in LMPPs. T-lineage cells were observed from TCF-1-expressing wild-type LMPPs on OP9-DL4 stroma, as expected, and ectopic TCF-1 rescued T-cell development from TCF-1^{-/-} progenitors (FIG. 2A and FIG. 3B). Ectopic TCF-1 and Notch1 signals together enhanced T-cell development (FIG. 9). However, when TCF-1-expressing progenitors were placed on OP9 stromal cells lacking Notch ligands, the development of T-lineage cells was also observed; this population was absent from progenitors transduced with control virus cultured on OP9 stroma (FIG. 2A). Ectopic expression of TCF-1 also efficiently inhibited the development of the B lineage but not of myeloid cells (FIG. 2B). However, because Notch signals efficiently inhibited the development of B cells from TCF-1^{-/-} progenitors (FIG. 1E), other mechanisms apart from TCF-1 to enforce lineage commitment must exist.

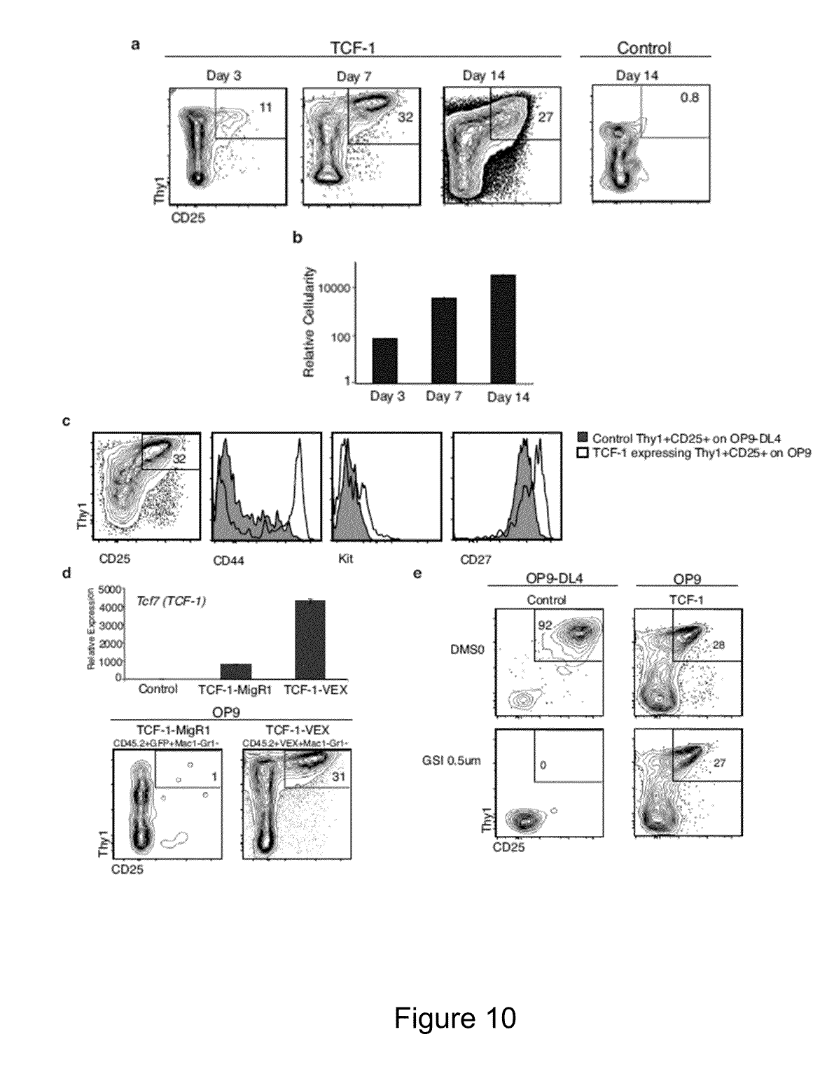

The TCF-1-mediated generation of Thy1⁺CD25⁺ cells on OP9 stroma was further investigated. These cells appeared early and expanded in number over time. They expressed surface markers of double-negative (DN) 2 and DN3 pro-T cell stages. A different retroviral vector that expresses TCF-1 at lower levels failed to generate Thy1⁺CD25⁺ cells, indicating a threshold level of TCF-1 expression is necessary. The generation of Thy1⁺CD25⁺ cells was unaffected by inhibitors of Notch signaling (FIG. 10). When injected intrathymically, these cells completed T-cell differentiation, reconstituting both αβ and γδ T-cell lineages (FIG. 2C).

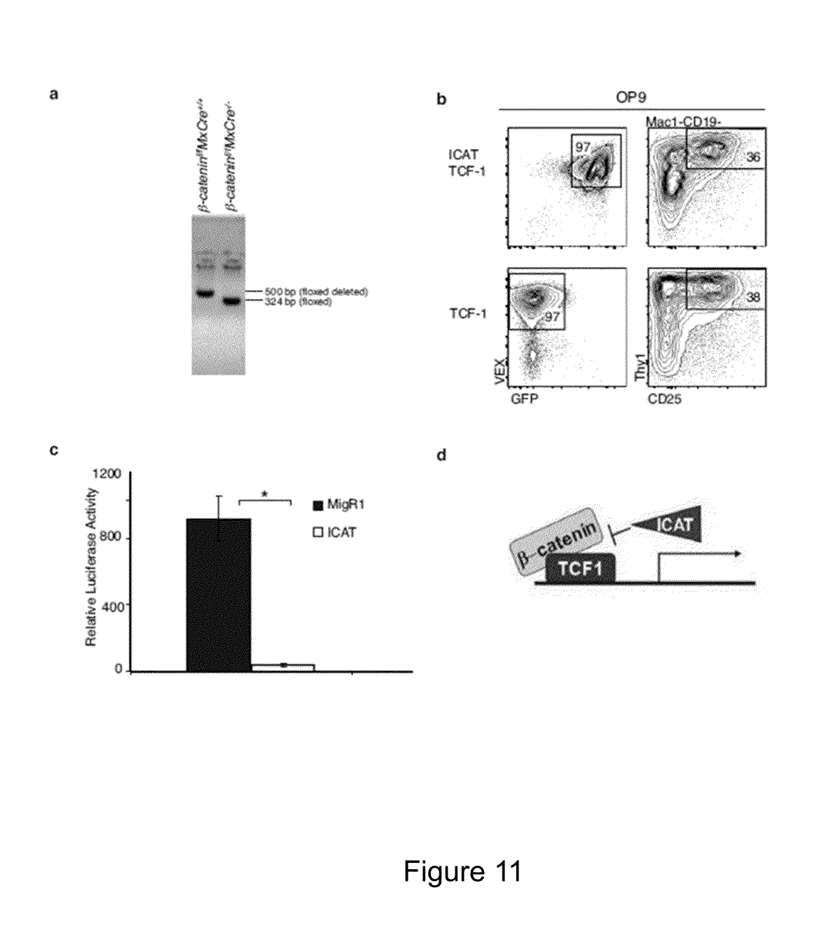

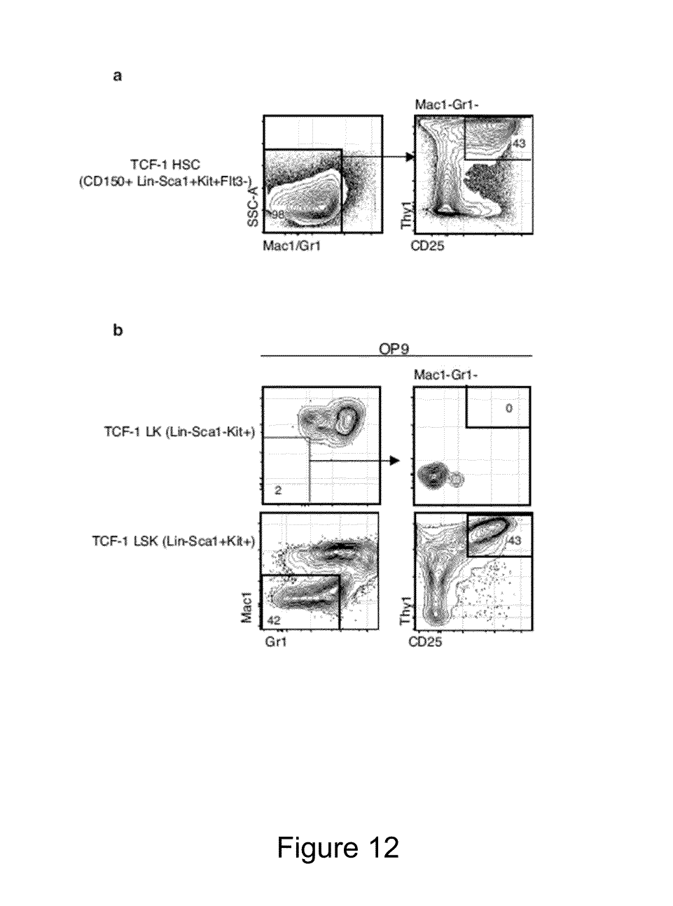

TCF-1 can function with β-catenin to mediate canonical Wnt signaling; however, deletion of β-catenin does not affect T-cell development (Cobas et al., 2004, J. Exp. Med., 199: 221-229; Jeannet et al., 2008, Blood, 111: 142-149). Consistently, the generation of Thy1⁺CD25⁺ cells was unaffected by deletion of β-catenin (FIG. 2D and FIG. 2E). Furthermore, ectopic expression of a small molecule inhibitor of β and γ-catenin, ICAT (Tago et al., 2000, Genes Dev., 14: 1741-1749), had no effect on the generation of TCF-1-expressing Thy1⁺CD25⁺ cells, demonstrating that TCF-1 is not acting as an effector of canonical Wnt signaling in early T-cell development (FIG. 11). Ectopic expression of TCF-1 in long-term hematopoietic stem cells (HSCs), but not in myelo-erythroid progenitors, resulted in development of T-lineage cells on OP9 stroma, indicating that TCF-1-directed T-lineage development is not restricted to lymphoid-biased progenitors (FIG. 12A), and requires factors absent from committed myelo-erythroid progenitors (FIG. 8B). These results indicate that TCF-1 is sufficient to induce the development of primitive hematopoietic progenitors into cells phenotypically and functionally resembling early T-cell precursors.

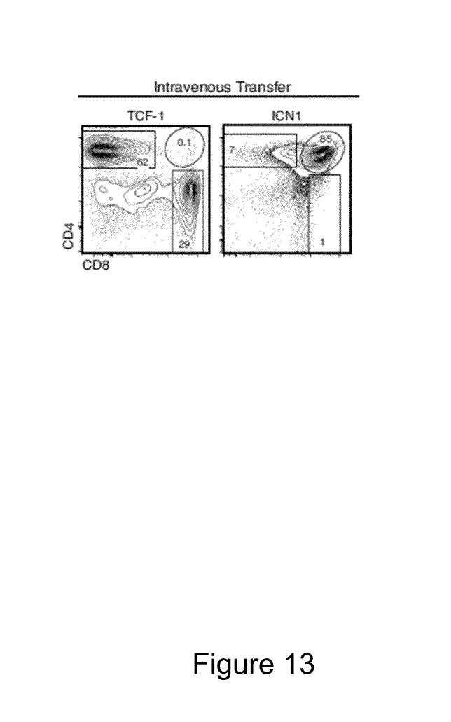

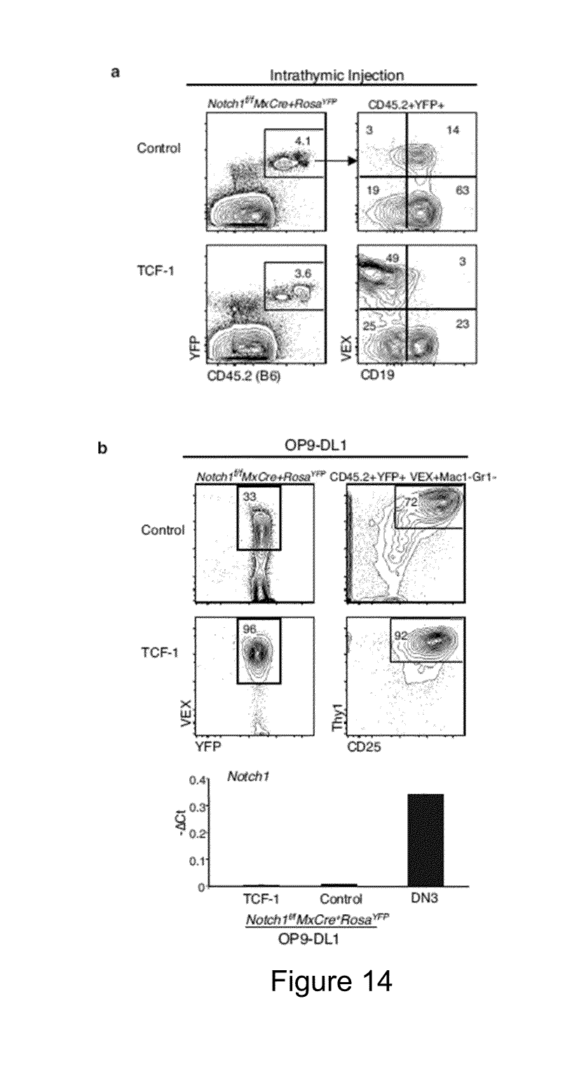

The effects of ectopic expression of TCF-1 in vivo were studied. When TCF-1-expressing progenitors were injected intravenously into irradiated mice, T-cell leukemia was not observed, unlike forced expression of intracellular Notch1 (ICN1) (FIG. 13) (Pear et al., 1996, J. Exp. Med., 183, 2283-2291). These data signify that key gene targets of ICN1 that control growth and oncogenesis are not similarly triggered by TCF-1. Next, TCF-1-expressing or control-vector-expressing progenitors from Notch1^{f/f}MxCre⁺Rosa^{YFP/+} mice that had been induced with polyinosinic:polycytidylic acid (poly(I:C)) were intrathymically injected. TCF-1-expressing progenitors lacking Notch1 gave rise to DN2/3-like Thy1⁺CD25⁺ cells, whereas control progenitors lacking Notch1 developed into B-lineage cells (FIG. 2D and FIG. 14). Therefore forced expression of TCF-1 can drive early T-cell development in the absence of Notch1 signals in the thymus.

To investigate the frequency of TCF-1-expressing LMPPs able to give rise to T-lineage cells, limiting dilution analysis was performed with TCF-1-expressing LMPPs on OP9 stromal cells and vector-control-expressing LMPPs on OP9-DL4. The frequencies of T-lineage cells developing in these cultures were similar (FIG. 2E). Thus, ectopic TCF-1 generates phenotypic T-cell precursors with frequencies comparable to Notch.

TCF-1 Directs T-Lineage Specification

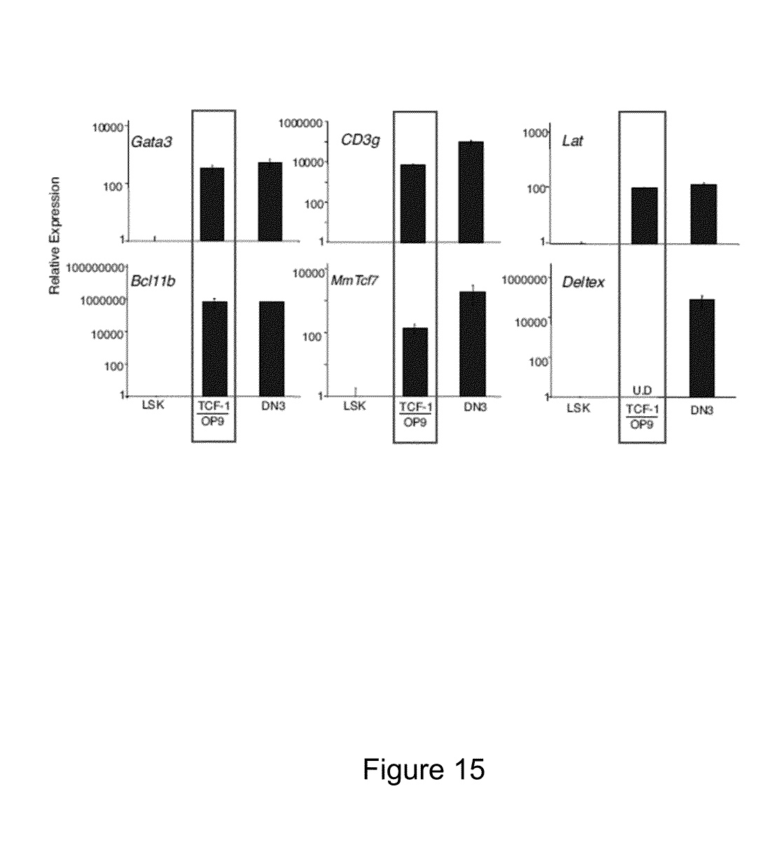

To understand whether TCF-1 is sufficient to direct a program of T-lineage-specific gene expression, global gene expression analysis was performed on TCF-1-expressing Thy1⁺CD25⁺ T-lineage cells that developed on OP9 stroma. Upregulated expression of many T-cell genes was found, including transcription factors Gata3 and Bcl11b, and T-cell structural genes including components of the T cell receptor (FIG. 3A). Established direct Notch1 gene targets such as Ptcra and Deltex1 (Deftos et al., 2000, Immunity, 13: 73-84) failed to be upregulated, confirming that these T-lineage cells arose independently of Notch1 signals (FIG. 3B). Quantitative PCR with reverse transcription (QRT-PCR) confirmed expression of key T-lineage genes, including Gata3, Bcl11b, CD3g, Lat, Lck and endogenous TCF-1 (FIG. 3B). At the time-points examined (day 10-day 14), expression of some genes in adult TCF-1-expressing Thy1⁺CD25⁺ cells was lower than the levels in DN3 thymocytes. Fetal liver progenitors show accelerated differentiation in vitro (Huang et al., 2005, J. Immunol., 175: 4858-4865); TCF-1-expressing Thy1⁺CD25⁺ cells from fetal liver consistently expressed T-cell genes at levels comparable to DN3 thymocytes by day 10 in culture (FIG. 15). However, some genes such as endogenous TCF-1 and CD3g never reached DN3 levels, suggesting additional regulatory inputs. These data indicate ectopic expression of TCF-1 drives expression of many T-cell lineage-specific genes.

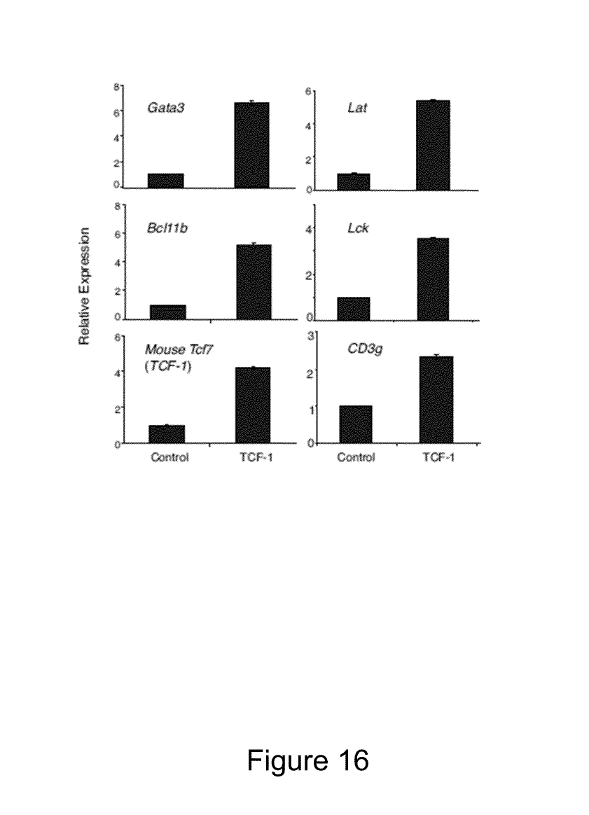

Analysis of T-lineage genes upregulated upon ectopic TCF-1 expression revealed many to contain evolutionarily conserved TCF-1 binding sites, suggesting a role for TCF-1 in directly regulating these genes. To validate these putative TCF-1 binding sites, chromatin immunoprecipitation assay (ChIP) was performed on CD4⁻CD8⁻ (DN) thymocytes with an antibody against TCF-1. It was found that TCF-1 was enriched at Gata3, Bcl11b, Il2ra, Cd3ε (also known as CD3e) and TCF-1 itself (FIG. 3C). In addition, T-lineage genes were already upregulated in TCF-1-expressing LSK progenitors (FIG. 16). Indeed, TCF-1 was initially cloned as a factor enriched at the CD3ε enhancer (van de Wetering et al., 1991, EMBO J., 10: 123-132) and TCF-1 has also been shown to regulate Gata3 in Th2 cells (Hosoya et al., 2009, J. Exp. Med., 206, 2987-3000). Gata3 is required in ETPs (Ikawa et al., 2010, Science, 329: 93-96), which may explain the paucity of ETPs from TCF-1^{-/-} progenitors. Bcl11b is critical for maintenance of T-lineage commitment, as deletion of Bcl11b in committed T-cells results in developmental arrest or diversion to the natural killer lineage (Ikawa et al., 2010, Science, 329: 93-96; Li et al., 2010, Science, 329: 89-93; Li et al., 2010, Science, 329: 85-89).

### Regulation of TCF-1

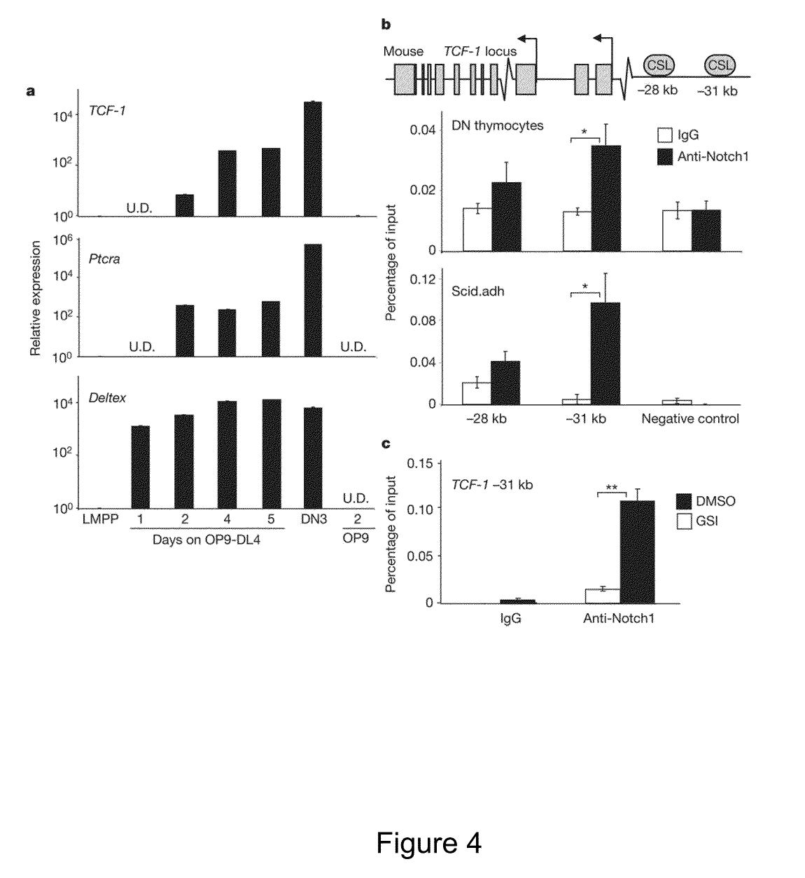

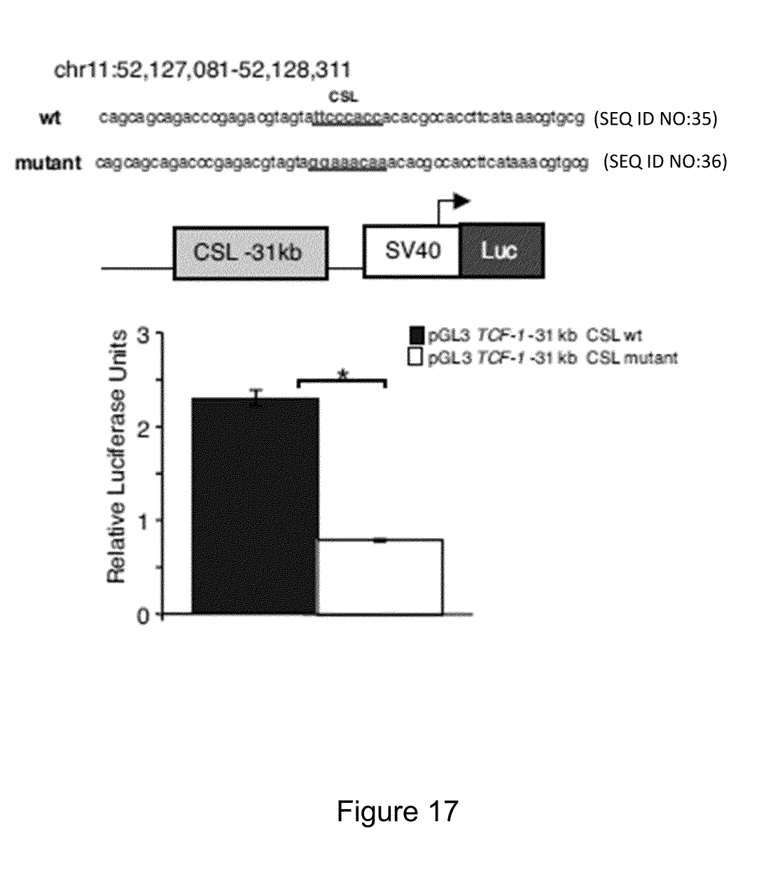

To examine how TCF-1 expression is initially upregulated by Notch signals, LMPPs were grown on OP9-DL4. Upregulated TCF-1 expression was found within 2 days that continued to rise over time, as expected (Taghon et al., 2005, Genes Dev., 19: 965-978) (FIG. 4A). ChIP revealed enrichment of Notch1 at a conserved −31 kilobases CSL (for CBF1, Suppressor of Hairless, and Lag-1) binding site in DN thymocytes and in ‘DN3-like’ Scid.adh cells (FIG. 4B); this binding was greatly decreased when Notch1 signals were blocked in vitro (FIG. 4C). The −31 kb CSL binding site was also active in a reporter assay (FIG. 17). These data indicate Notch1 regulates TCF-1 expression.

Although TCF-1 is initially expressed downstream of Notch1 signals, TCF-1 may also regulate its own expression. TCF-1 binds to the TCF-1 locus (FIG. 3D), and ectopic expression of human TCF-1 is sufficient to induce mouse TCF-1 gene expression (FIG. 3B). Consistently, it was found that TCF-1 activates a reporter containing the TCF-1 promoter; mutation of the TCF-1 binding site decreased activation (FIG. 3E). While not wishing to be bound by any particular theory, positive autoregulation may be one mechanism by which TCF-1 remains highly expressed after Notch1 signals cease after the β-selection checkpoint (Taghon et al., 2006, Immunity, 24: 53-64; Yashiro-Ohtani et al., 2009, Genes Dev., 23: 1665-1676), contributing to the stability of T-cell-specific gene expression.

### TCF-1 is a Critical Regulator of T-Cell Development and Identity

In B cells, a network of transcription factors composed of E47, EBF1, FoxO1 and Pax5 drives B-lineage gene expression (Lin et al., 2010, Nature Immunol., 11: 635-343). For T-cells, similar factors were previously unknown; the present study implicates TCF-1 in this role. The results presented herein demonstrate a model in which TCF-1 is induced by Notch signals in ETPs, and subsequently TCF-1 drives T-cell lineage specification. Among the genes induced by TCF-1 are components of the TCR, as well as T-cell essential transcription factors Gata3 and Bcl11b. Without wishing to be bound by any particular theory, the data described herein are consistent with TCF-1 having a role in inhibiting the B-cell fate early in T-cell development, although redundant mechanisms to inhibit B-cell development from ETPs must also exist (Wendorff et al., 2010, Immunity, 33: 671-684). The present study establishes TCF-1 as a critical regulator that is not only essential for normal T-cell development but is sufficient to establish many components of T-cell identity.

TABLE 1
List of primers used in this study

| | |
|---|---|
| qPCR primers | Primer Sequence (5′ to 3′) |

TCF-1 ChIP

| | | |
|---|---|---|
| Cd3e-3′ enhancer-Fwd | CGTTCATGTGCCTTGTGTGT | SEQ ID NO: 1 |
| Cd3e-3′ enhancer-Rev | TCATTGCAGTGCTTCCTGTT | SEQ ID NO: 2 |
| CD3e-Negcontrol-Fwd | TCTCTTGACTTCTGGCAGAGC | SEQ ID NO: 3 |
| CD3e-Negcontrol-Rev | GTGTGAGCCGAAAGAAAAGG | SEQ ID NO: 4 |
| Axin2-3 kb-Fwd | TTAAAGCGCCTCTGTGATTG | SEQ ID NO: 5 |
| Axin2-3 kb-Rev | CGCGAACGGCTGCTTATT | SEQ ID NO: 6 |
| Tcf7-1.3 kb-Fwd | TTTGTGAAGGAGGACACTGG | SEQ ID NO: 7 |
| Tcf7-1.3 kb-Rev | CTGAGCGCTGAGAAGCAAG | SEQ ID NO: 8 |
| Tcf7-4 kb-Fwd | AAATCATCCGACCGTTCTCA | SEQ ID NO: 9 |
| Tcf7-4 kb-Rev | AGGATCTCCCGGTTAGGAAA | SEQ ID NO: 10 |
| Bcl1 1b-2 kb-Fwd | GTCGTCCCCCTCCTCCAT | SEQ ID NO: 11 |
| Bcl1 1b-2 kb-Rev | ACTGCAGCCTGGCCTTGT | SEQ ID NO: 12 |
| Gata3-1.7 kb-Fwd | GGGAAAGCAAGCAGAGACCA | SEQ ID NO: 13 |
| Gata3-1.7 kb-Rev | TTGCCTCCGAACCAGCTTTC | SEQ ID NO: 14 |
| CD25-6 kb-Fwd | TTCAGAGCCCAGTGTAAGAGC | SEQ ID NO: 15 |
| CD25-6 kb-Rev | TGTCTATCAATGTCTTGAGAAGTCTAC | SEQ ID NO: 16 |

Notch1 ChIP

| | | |
|---|---|---|
| Tcf7-CSL-28 kb-Fwd | CATTTTGCATCTGGGCTACA | SEQ ID NO: 17 |
| Tcf7-CSL-28 kb-Rev | GGATGCCAGTCAAGGAAAAT | SEQ ID NO: 18 |
| Tcf7-CSL-31 kb-Fwd | CGAACCCCAGCAAGAAATAG | SEQ ID NO: 19 |
| Tcf7-CSL-31 kb-Rev | ACAAAGCGACCACAGCTTTT | SEQ ID NO: 20 |
| Tcf7-CSL-negcontrol- | CCCCCTGCTCTCTCTGTATG | SEQ ID NO: 21 |
| Fwd | | |
| Tcf7-CSL-negcontrol-Rev | ATGAACACATTTGCCACAGC | SEQ ID NO: 22 |

Genotyping Primers

| | | |
|---|---|---|
| Tcf7-exon7-Fwd | ACCTTTTCACCCCAGCTTTC | SEQ ID NO: 23 |
| Tcf7-exon7-Rev | ATTCCCCTTCCTGTGTTGAG | SEQ ID NO: 24 |
| Tcf7-Pn5B | CTAAAGCGCATGCTCCAGACT | SEQ ID NO: 25 |
| Notch1f/f-Fwd | TGCCCTTTCCTTAAAAGTGG | SEQ ID NO: 26 |
| Notch1f/f-Rev | GCCTACTCCGACACCCAATA | SEQ ID NO: 27 |
| Beta catenin-Rm41-Fwd | AAGGTAGAGTGATGAAAGTTGTT | SEQ ID NO: 28 |
| Beta catenin-Rm42-Rev | CACCATGTCCTCTGTCTATTC | SEQ ID NO: 29 |
| Beta catenin-Rm43-Fwd | TACACTATTGAATCACAGGGACTT | SEQ ID NO: 30 |

RT-PCR primers (Unless stated here, all other genes were detected
with Taqman probes)

| | | |
|---|---|---|
| HuMmTcf7-Fwd | AGGAGATGAGAGCCAAGGTCATTG | SEQ ID NO: 31 |
| HuMmTcf7-Rev | TTTTCCTCCTGTGGTGGATTCTTG | SEQ ID NO: 32 |
| TCRg-V3-Fwd | TGCCTCTTGACATTTGGACA | SEQ ID NO: 33 |
| TCRg-V3 -Rev | GTTTCTGCCGGTACCAGTGT | SEQ ID NO: 34 |

TABLE 2
Two Fold gene lists from FIG. 1E

| | Tcf1^{+/+} lineage negative | | Tcf1^{-/-} lineage negative | |
|---|---|---|---|---|
| | to Tcf1^{+/+} Thy1⁺ CD25⁺ | | toTcf1^{-/+} Thy1⁺ CD25⁺ | |
| | | Fold | | Fold |
| Gene symbol | p-value | change | p-value | change |
| II2ra | 0.00150553 | 38.2994 | 0.000886685 | 77.8214 |
| Cd3g | 0.00396569 | 66.4554 | 0.00560987 | 55.2367 |
| H19 | 0.00327325 | 20.9391 | 0.00413762 | 22.688 |
| Thy1 | 0.00414442 | 13.4071 | 0.00553657 | 13.2003 |
| Plxdc2 | 0.00396569 | 9.76399 | 0.00481203 | 12.5921 |
| Fam20a | 0.016421 | 11.7184 | 0.0205699 | 12.0765 |
| Tcf7 | 0.00150563 | 7.24097 | 0.000898665 | 10.2718 |
| Khdc1a | 0.00163744 | 9.87948 | 0.00150487 | 8.90815 |
| Akr1c12 | 0.00692664 | 8.66694 | 0.00802261 | 8.77419 |
| Rag1 | 0.0177877 | 9.98093 | 0.0251491 | 8.76263 |
| Gzma | 0.00214638 | 9.44172 | 0.00286822 | 8.47202 |
| Ppic | 0.00711607 | 7.23859 | 0.00910223 | 7.36651 |
| Esm1 | 0.00156583 | 6.55657 | 0.00150467 | 7.18497 |
| Khdc1a | 0.00378337 | 6.94018 | 0.00474476 | 7.03651 |
| Cd3d | 0.0070292 | 7.30749 | 0.049386 | 6.68053 |
| A6300138E17RlK | 0.00388911 | 6.54783 | 0.00496816 | 6.49016 |
| Khdc1b | 0.00277886 | 5.939591 | 0.00297244 | 6.00702 |
| Gzmb | 0.00281901 | 5.01813 | 0.00297244 | 6.75184 |
| Trat1 | 0.0280714 | 5.68658 | 0.0329848 | 6.59342 |
| Tubb3 | 0.0080051 | 4.61485 | 0.0287732 | 6.52619 |
| Cpa3 | 0.01993 | 5.22772 | 0.0236131 | 5.42861 |
| Sytl3 | 0.00734747 | 4.96518 | 0.00868337 | 5.41409 |
| Lat | 0.00476035 | 4.87632 | 0.00611982 | 5.27851 |
| Fam169b | 0.00373086 | 6.05441 | 0.0104523 | 5.24574 |
| Gm4827 | 0.0160913 | 4.62456 | 0.0212223 | 5.12475 |
| Psg17 | 0.0112938 | 4.33281 | 0.0132658 | 4.62012 |
| Akr1c13 | 0.00491928 | 4.83568 | 0.00726395 | 4.55175 |
| Ctla4 | 0.027889 | 4.77572 | 0.0361514 | 4.48349 |
| Cd160 | 0.0116984 | 3.28632 | 0.00902261 | 4.44302 |
| Prkcq | 0.00823894 | 5.44673 | 0.0107804 | 4.41367 |
| Stk39 | 0.00911994 | 4.18337 | 0.0124078 | 4.01837 |
| Txk | 0.0013943 | 3.77677 | 0.0368402 | 3.91818 |
| Tnfsf10 | 0.0110426 | 4.62501 | 0.0188152 | 3.91739 |
| Muc13 | 0.005644899 | 4.34653 | 0.00938953 | 3.76571 |
| Arpp21 | 0.0168545 | 3.62712 | 0.0221932 | 3.59017 |
| Gimap1 | 0.0414729 | 3.41208 | 0.464341 | 3.63799 |
| II17rb | 0.0163649 | 3.66105 | 0.014305 | 2.54381 |
| Sestd1 | 0.00387872 | 2.66189 | 0.00297244 | 3.41858 |
| Naip3 | 0.00414442 | 2.47717 | 0.00323591 | 2.41332 |
| Adamts3 | 0.00327172 | 3.70588 | 0.00455621 | 3.35704 |
| Prkca | 0.0708297 | 2.65884 | 0.0548791 | 2.30078 |
| Slc22a3 | 0.005887856 | 3.57982 | 0.0139737 | 3.2395 |
| Mpp4 | 0.0118514 | 3.06271 | 0.01401 | 3.23012 |
| E030002O03Rik | 0.0157658 | 3.47217 | 0.22654 | 3.22259 |
| 1190002H23Rik | 0.0280304 | 2.95123 | 0.0268535 | 3.21378 |
| Xrcc5 | 0.00327325 | 3.57866 | 0.00482762 | 3.16698 |
| Gimap9 | 0.0143313 | 2.81319 | 0.0147111 | 2.14645 |
| Adamts3 | 0.0265995 | 3.51919 | 0.458652 | 3.11672 |
| Gm6683 | 0.003189 | 2.86974 | 0.34934 | 3.11037 |
| Cxcr5 | 0.00658826 | 3.61568 | 0.011348 | 3.09281 |
| Tuba8 | 0.00533663 | 3.41419 | 0.00672093 | 3.09022 |
| Armcx4 | 0.0023493 | 2.856027 | 0.0240721 | 3.07745 |
| Gm13926 | 0.00449176 | 2.84616 | 0.00482782 | 3.03774 |
| Vat1l | 0.00673627 | 3.16111 | 0.00940567 | 3.02695 |
| Gm5111 | 0.020359 | 3.29363 | 0.0300998 | 3.02269 |
| Gata3 | 0.0142867 | 3.19352 | 0.0212866 | 2.96663 |
| Dnahc8 | 0.00636105 | 2.92012 | 0.106286 | 2.90265 |
| F2r | 0.0070842 | 2.8153 | 0.00902281 | 2.86991 |
| Cpne2 | 0.0684659 | 2.71331 | 0.659239 | 2.84635 |
| Lif | 0.00342238 | 3.12348 | 0.00503469 | 2.82309 |
| Tcrg-V3 | 0.00524684 | 2.10355 | 0.00423568 | 2.8042 |
| Ccr4 | 0.0354161 | 2.58581 | 0.365452 | 2.79638 |
| A130014H13Rik | 0.254626 | 2.61579 | 0.294708 | 2.78153 |
| Mfsd2 | 0.0114308 | 2.80983 | 0.0131137 | 2.75419 |
| Adamts3 | 0.0134211 | 3.22885 | 0.0224355 | 2.75276 |
| Clec4e | 0.00741865 | 2.97519 | 0.0115854 | 2.72458 |
| Ets1 | 0.0267514 | 2.44743 | 0.25723 | 2.71659 |
| Nck2 | 0.00327172 | 2.67158 | 0.00401518 | 2.71337 |
| Gimap5 | 0.0182823 | 2.46679 | 0.0189906 | 2.89739 |
| Adamts3 | 0.00731341 | 2.58407 | 0.0067838 | 2.6929 |
| Sytl2 | 0.0892283 | 2.66215 | 0.51259 | 2.8141 |
| Podh7 | 0.00836105 | 2.97738 | 0.0147111 | 2.60968 |
| B4Galnt2 | 0.00396569 | 2.79732 | 0.00611982 | 2.60538 |
| Ndrg2 | 0.728253 | 2.69628 | 0.115782 | 2.56413 |
| Kbtbd11 | 0.0381592 | 2.18011 | 0.029435 | 2.58053 |
| 4933415A04Rik | 0.0114029 | 2.4141 | 0.0124078 | 2.57437 |
| Itga2b | 0.00521472 | 2.49107 | 0.00703767 | 2.51199 |
| Ctla2b | 0.0257271 | 2.78391 | 0.419153 | 2.49894 |
| Entpd5 | 0.0197741 | 2.62776 | 0.0264614 | 2.8441 |
| Onmt3b | 0.0134196 | 2.62382 | 0.0198683 | 2.47959 |
| Rasgrp1 | 0.678147 | 2.55277 | 0.0907044 | 2.47948 |
| Lrp12 | 0.00516306 | 2.75441 | 0.00893189 | 2.46335 |
| Sstr2 | 0.0282152 | 2.88354 | 0.463869 | 2.45579 |
| AI847670 | 0.00265423 | 2.325071 | 0.00297244 | 2.42505 |
| Irgm2 | 0.00317962 | 1.95232 | 0.00280101 | 2.42565 |
| Tox | 0.068171 | 2.10265 | 0.0462551 | 2.41917 |
| Rag2 | 0.391207 | 2.92588 | 0.079532 | 2.40243 |
| Lgals9 | 0.011859 | 2.79428 | 0.0219571 | 2.3974 |
| 2900006K08Rik | 0.0157658 | 2.18191 | 0.0152422 | 2.37182 |
| Svil | 0.00517268 | 2.31309 | 0.0067748 | 2.36873 |
| A630091E08Rik | 0.158758 | 2.14842 | 0.151985 | 2.38161 |
| Aqp11 | 0.02276 | 2.56104 | 0.0361745 | 2.3554 |
| D330050I23Rik | 0.0142867 | 2.21835 | 0.0154339 | 2.35497 |
| Zip709 | 0.0638455 | 1.72646 | 0.0232868 | 2.35333 |
| Fam40b | 0.0960647 | 2.76191 | 0.17674 | 2.34587 |
| Epcam | 0.0104322 | 2.40528 | 0.0147111 | 2.39935 |
| Tmtc2 | 0.142092 | 2.15574 | 0.140742 | 2.33418 |
| 2btb16 | 0.0030598 | 2.12605 | 0.0320952 | 2.30356 |
| Fbp1 | 0.0558894 | 2.34537 | 0.653605 | 2.29879 |
| Lck | 0.00813889 | 2.86125 | 0.014251 | 2.29094 |
| Hck2 | 0.00357933 | 2.03389 | 0.00336178 | 2.29079 |
| Irak3 | 0.868219 | 2.24152 | 0.098889 | 2.28957 |
| AA487197 | 0.157634 | 2.16674 | 0.16704 | 2.27765 |
| Cpp4 | 0.0236635 | 2.3103 | 0.0308824 | 2.26886 |
| Cank2d | 0.0718744 | 2.8018 | 0.0914124 | 2.28269 |
| Endh | 0.463016 | 1.64885 | 0.030388 | 2.26196 |
| Ak2 | 0.0192058 | 2.02109 | 0.0182719 | 2.25324 |
| 2610020H05Rik | 0.0296587 | 2.64326 | 0.0267671 | 2.24574 |
| Rab44 | 0.00414442 | 2.28949 | 0.00629113 | 2.23835 |
| Bd211 | 0.204908 | 2.39823 | 0.0311989 | 2.23477 |
| Med121 | 0.0070642 | 2.22363 | 0.0092128 | 2.21908 |
| Adamts3 | 0.0187973 | 2.29468 | 0.0274993 | 2.16129 |
| Cgke | 0.0864038 | 2.07313 | 0.0395325 | 2.16084 |
| Adamis9 | 0.028242 | 2.20452 | 0.0374741 | 2.16057 |
| 2818001F02Rik | 0.112571 | 2.15109 | 0.138241 | 2.15538 |
| 4930520O841Rik | 0.439397 | 1.88364 | 0.0421967 | 2.15468 |
| Fgl3 | 0.0145568 | 2.07367 | 0.017025 | 2.14453 |
| Cnajc6 | 0.0100554 | 2.78272 | 0.0251868 | 2.13835 |
| Slc15a1 | 0.128455 | 2.63351 | 0.135638 | 2.13781 |
| Ctla2a | 0.19125 | 2.26084 | 0.0287405 | 2.1331 |
| Ggr58 | 0.04057 | 2.58752 | 0.0734582 | 2.12756 |
| Hemge | 0.0714083 | 2.14923 | 0.0918059 | 2.1248 |
| Cdk81a | 0.0198365 | 2.26372 | 0.0300988 | 2.11685 |
| Kenk5 | 0.0208959 | 2.32081 | 0.358918 | 2.10258 |
| Trim13 | 0.00906153 | 1.90049 | 0.00914343 | 2.10209 |
| Neth | 0.129427 | 2.14002 | 0.0190363 | 2.0975 |
| Cdx3y | 0.552978 | −1.48423 | 0.87804 | 2.07215 |
| Ral125 | 0.135128 | 2.30298 | 0.210891 | 2.05975 |
| Adamts3 | 0.00499947 | 2.18351 | 0.0081728 | 2.0505 |
| Myon | 0.048907 | 2.12785 | 0.0675718 | 2.04715 |
| Gm12250 | 0.0073164 | 1.80958 | 0.0268174 | 2.04809 |
| Gre | 0.178533 | 1.61435 | 0.0149624 | 2.04329 |
| Actr3b | 0.22144 | 2.06689 | 0.269594 | 2.04064 |
| Dnahe8 | 0.0260142 | 1.97839 | 0.296086 | 2.0232 |
| Inpp4b | 0.0956938 | 1.95201 | 0.104327 | 2.02198 |
| Hlvep22 | 0.0341278 | 2.04891 | 0.0443725 | 2.01854 |
| Pp1tr | 0.065397 | 2.37314 | 0.129275 | 2.01615 |
| Pkcr1 | 0.0043549 | 1.80927 | 0.0284614 | 2.01475 |
| Gpr87 | 0.0749238 | 2.21012 | 0.123015 | 2.01048 |
| Crp1 | 0.016421 | 2.07368 | 0.022664 | 2.01009 |
| Aspth | 0.031508 | 1.79318 | 0.0258874 | 2.00913 |
| Rag2 | 0.0564862 | 2.23905 | 0.0991575 | 2.00745 |
| H2-Aa | 0.002828 | −41.3805 | 0.003236 | −38.7481 |
| Klrd1 | 0.001506 | −52.231 | 0.001804 | −37.8262 |
| H2-Ab1 | 0.002346 | −28.0605 | 0.002972 | −26.724 |
| Nucb2 | 0.00349 | −30.1197 | 0.004828 | −24.5633 |
| Ifl205 | 0.002818 | −22.919 | 0.003236 | −22.0299 |
| Mpeg1 | 0.002826 | −22.1697 | 0.003444 | −20.4158 |
| Ctsh | 0.002348 | −23.8919 | 0.002972 | −18.9326 |
| Clec9a | 0.003257 | −23.2537 | 0.004427 | −18.8304 |
| Cybb | 0.003272 | −24.3794 | 0.004718 | −17.5653 |
| Rnd3 | 0.004428 | −19.6849 | 0.006775 | −17.0234 |
| Plbd1 | 0.00257 | −15.9281 | 0.002972 | −15.5872 |
| Hppd | 0.003988 | −23.6862 | 0.006775 | −15.2337 |
| S100a8 | 0.010085 | −23.1208 | 0.018594 | −15.0602 |
| Kynu | 0.003688 | −21.7617 | 0.006834 | −14.211 |
| All1 | 0.001586 | −13.5258 | 0.001506 | −14.0974 |
| Cd74 | 0.003798 | −16.2745 | 0.005366 | −14.8784 |
| Clss | 0.007947 | −16.3196 | 0.011416 | −14.0146 |
| Flt3 | 0.002194 | −16.0217 | 0.002972 | −13.675 |
| Map4kS | 0.002894 | −14.9674 | 0.002972 | −13.5688 |
| Kirk1 | 0.002961 | −15.2832 | 0.004016 | −12.9827 |
| Ms4a4c | 0.004144 | −15.1293 | 0.008903 | −11.8751 |
| Dirc2 | 0.00188 | −14.4243 | 0.002801 | −11.2249 |
| Sigtech | 0.003273 | −16.2656 | 0.005327 | −11.1681 |
| Ms4a6c | 0.006901 | −11.6164 | 0.009431 | −10.7671 |
| Tlr11 | 0.006123 | −11.856 | 0.009023 | −10.4568 |
| Cst1r | 0.003273 | −10.1809 | 0.004236 | −10.2796 |
| Rnase6 | 0.002348 | −12.3544 | 0.002972 | −10.0486 |
| Lyz2 | 0.008995 | −13.8619 | 0.016379 | −9.34991 |
| Itgax | 0.001588 | −8.73566 | 0.001506 | −9.252 |
| Slamf7 | 0.004144 | −13.1915 | 0.007333 | −9.21189 |
| H2-Eb1 | 0.002667 | −10.041 | 0.003136 | −9.06281 |
| S100a9 | 0.012709 | −15.7637 | 0.027648 | −8.62686 |
| Kmo | 0.005948 | −8.25372 | 0.006863 | −8.54477 |
| Ms4s3 | 0.27071 | −7.93667 | 0.031067 | −8.4761 |
| Gm6377 | 0.009986 | −8.16887 | 0.011981 | −8.45041 |
| Ly66 | 0.002527 | −9.89155 | 0.002972 | −8.40732 |
| Tcfec | 0.005753 | −8.26036 | 0.009006 | −8.25132 |
| Scpep1 | 0.002348 | −10.1578 | 0.002972 | −7.98684 |
| Cd7 | 0.00188 | −6.47416 | 0.002332 | −7.92266 |
| Entpd1 | 0.004084 | −6.71077 | 0.004828 | −7.70758 |
| Irf8 | 0.005240 | −8.08352 | 0.007433 | −7.63953 |
| Eltd1 | 0.011115 | −5.48005 | 0.009569 | −7.40736 |
| Pyhin1 | 0.006586 | −6.91348 | 0.010424 | −7.33177 |
| Lifr | 0.002618 | −11.8273 | 0.004748 | −7.09463 |
| AI607673 | 0.013146 | −9.04695 | 0.022148 | −6.97433 |
| Crybg3 | 0.004021 | −7.9902 | 0.006258 | −6.83999 |
| Samd9I | 0.004144 | −10.0788 | 0.008425 | −6.83714 |
| Pira2 | 0.003968 | −8.5909 | 0.007937 | −6.7716 |
| Cd86 | 0.013801 | −6.32439 | 0.018514 | −6.72671 |
| Lgmn | 0.004144 | −7.38008 | 0.006491 | −6.54146 |
| Fam129a | 0.003273 | −6.58795 | 0.004512 | −6.49673 |
| Bd2a1a | 0.005388 | −8.42536 | 0.007264 | −6.48643 |
| Gm5431 | 0.018991 | −7.56008 | 0.036043 | −6.47408 |
| II1r1 | 0.063722 | −5.09771 | 0.69309 | −6.42722 |
| Jhdm1d | 0.007312 | −7.21145 | 0.010992 | −6.30218 |
| Lmk25 | 0.003273 | −7.12896 | 0.004555 | −6.28671 |
| Plxnc1 | 0.001995 | −7.41698 | 0.002801 | −6.24838 |
| 5430435G22Rik | 0.017916 | −8.51667 | 0.022736 | −6.23562 |
| Gm11428 | 0.011908 | −11.7381 | 0.030043 | −6.18904 |
| Ifrtm6 | 0.018169 | −8.81849 | 0.022865 | −6.14944 |
| Cd300a | 0.001586 | −8.25276 | 0.001536 | −6.07857 |
| Amica1 | 0.003949 | −7.11374 | 0.006238 | −5.93143 |
| Picn | 0.004633 | −8.64706 | 0.007163 | −5.86016 |
| 9930111J21Rik | 0.002429 | −6.21472 | 0.002972 | −5.78522 |
| Gm6455 | 0.004144 | −3.69591 | 0.003236 | −5.72349 |
| Gm6455 | 0.004144 | −3.57295 | 0.003238 | −5.71638 |
| Btla | 0.004623 | −7.48254 | 0.008553 | −5.69722 |
| Sp100 | 0.001995 | −8.33755 | 0.002601 | −5.69143 |
| 4930506M07Rik | 0.01474 | −6.72401 | 0.019819 | −5.62395 |
| H2-DMb2 | 0.022826 | −6.96368 | 0.31141 | −5.55408 |
| Bd2a1b | 0.007668 | −5.4494 | 0.009627 | −5.50335 |
| Bd2a1d | 0.00759 | −5.43594 | 0.009578 | −5.48018 |
| Jhdm1d | 0.003624 | −8.34451 | 0.005261 | −5.45747 |
| Hpse | 0.002836 | −5.13558 | 0.002972 | −5.42051 |
| Csf2rb | 0.007698 | −5.33738 | 0.009143 | −5.3832 |
| Ccl3 | 0.007016 | −4.49577 | 0.007269 | −5.34796 |
| Alm1 | 0.008685 | −8.14892 | 0.010285 | −5.32893 |
| Klra17 | 0.016501 | −10.1823 | 0.044936 | −5.29643 |
| Pgap1 | 0.014982 | −7.12562 | 0.026362 | −5.148017 |
| Id2 | 0.004721 | −5.1818 | 0.005634 | −5.13053 |
| Xcr1 | 0.15466 | −4.93994 | 0.1805 | −5.10178 |
| Raet1b | 0.018988 | −4.99093 | 0.023883 | −4.93076 |
| Psap | 0.002346 | −4.85014 | 0.002972 | −4.82013 |
| Lrro4c | 0.10375 | −4.2634 | 0.106739 | −4.79562 |
| Cd300c | 0.005249 | −6.54083 | 0.010244 | −4.77182 |
| Anpep | 0.009097 | −4.86297 | 0.010827 | −4.73687 |
| Rassf4 | 0.003795 | −6.07206 | 0.006495 | −4.84289 |
| Mef2c | 0.01365 | −4.57354 | 0.17869 | −4.59016 |
| Pira11 | 0.00481 | −6.26479 | 0.009143 | −4.52087 |
| Csf2rb2 | 0.004144 | −5.41831 | 0.007037 | −4.5027 |
| Klri2 | 0.009475 | −5.88818 | 0.018274 | −4.49185 |
| Ceacam1 | 0.009923 | −5.61884 | 0.016255 | −4.4546 |
| Pgap1 | 0.021293 | −6.95176 | 0.05078 | −4.41707 |
| Cadm1 | 0.003556 | −5.95435 | 0.006258 | −4.40299 |
| 9030625A04Rik | 0.00912 | −4.86379 | 0.013974 | −4.38088 |
| Sat1 | 0.004144 | −4.68401 | 0.006258 | −4.3181 |
| 9030420J04Rik | 0.023273 | −4.91678 | 0.038269 | −4.30787 |
| Fkbp lb | 0.002628 | −5.38518 | 0.004512 | −4.30318 |
| Cd36 | 0.015821 | −3.73511 | 0.016031 | −4.26973 |
| Prdx4 | 0.007084 | −4.92345 | 0.0113 | −4.25665 |
| Gpr137b-ps | 0.007634 | −4.63811 | 0.011962 | −4.2494 |
| Gm10759 | 0.016883 | −4.48665 | 0.025562 | −4.22185 |
| Tet1 | 0.004149 | −4.16724 | 0.005884 | −4.22072 |
| Speert-ps1 | 0.014294 | −2.74292 | 0.008474 | −4.20164 |
| Slfn8 | 0.003973 | −5.5814 | 0.007333 | −1.19543 |
| Ccr2 | 0.004144 | −6.06602 | 0.007284 | −4.18688 |
| Alpk1 | 0.003415 | −4.61072 | 0.004826 | −4.16975 |
| Evi5 | 0.002826 | −4.96494 | 0.004236 | −4.15601 |
| Cytip | 0.003795 | −4.22501 | 0.004826 | −4.016107 |
| Ahnak | 0.003889 | −5.18993 | 0.008634 | −4.13408 |
| Fam135a | 0.005408 | −4.48062 | 0.0068 | −4.08134 |
| Slc44a1 | 0.018991 | −5.63533 | 0.038707 | −4.07409 |
| Anxa3 | 0.005339 | −4.18007 | 0.007609 | −4.06192 |
| Naaa | 0.009949 | −4.70014 | 0.014711 | −4.05911 |
| Id3 | 0.00698 | −4.57047 | 0.01078 | −4.03959 |
| Tlr3 | 0.018683 | −3.91502 | 0.022468 | −3.99285 |
| Slfn5 | 0.00947 | −4.86741 | 0.016914 | −3.99925 |
| Afcam | 0.003272 | −4.48287 | 0.004512 | −3.9864 |
| Cd33 | 0.008073 | −3.17879 | 0.007287 | −3.94696 |
| Anxa5 | 0.003272 | −4.33214 | 0.004512 | −3.93169 |
| Gafm | 0.018989 | −3.82565 | 0.023219 | −3.90361 |
| Gpr137b | 0.007668 | −4.70041 | 0.013688 | −3.89535 |
| Slc9a7 | 0.002755 | −4.19058 | 0.003236 | −3.85672 |
| Rasgrp3 | 0.032319 | −4.18157 | 0.046868 | −3.85018 |
| H2-Cb | 0.006293 | −3.44141 | 0.007037 | −3.84337 |
| Myo9a | 0.138291 | −4.04577 | 0.100805 | −3.64335 |
| Anxa1 | 0.011115 | −4.58428 | 0.019528 | −3.62873 |
| Cd180 | 0.028562 | −4.30343 | 0.043739 | −3.82788 |
| Adrbk2 | 0.014697 | −3.84802 | 0.019709 | −3.79258 |
| Tita | 0.012633 | −4.33731 | 0.019709 | −3.77552 |
| H2-DMa | 0.017275 | −3.58121 | 0.020358 | −3.75258 |
| Tgm3 | 0.069338 | −3.13068 | 0.061569 | −3.7483 |
| Tel1 | 0.000083 | −3.99019 | 0.041915 | −3.73799 |
| Pid4 | 0.028735 | −4.57841 | 0.051049 | −3.72409 |
| Tlr7 | 0.021753 | −4.83558 | 0.042331 | −3.71892 |
| Grr6455 | 0.038835 | −2.39854 | 0.019619 | −3.87454 |
| Ccr5 | 0.005408 | −4.48333 | 0.009669 | −3.65801 |
| Ccr5 | 0.005408 | −4.46333 | 0.009689 | −3.85801 |
| Hpgds | 0.028491 | −3.71292 | 0.036845 | −3.85347 |
| Bird1f | 0.01474 | −4.14247 | 0.023222 | −3.62773 |
| Tet1 | 0.021208 | −3.84263 | 0.029938 | −3.60799 |
| Tceal1 | 0.005163 | −3.28681 | 0.006258 | −3.59017 |
| Fam49s | 0.006468 | −3.69231 | 0.009023 | −3.67516 |
| Xir | 0.000288 | −3.16152 | 0.079532 | −3.55693 |
| Ifngr2 | 0.002343 | −4.20214 | 0.00316 | −3.5198 |
| 503141D18Rik | 0.002826 | −4.21636 | 0.004468 | −3.50595 |
| Syna2 | 0.004144 | −6.06824 | 0.012042 | −3.49613 |
| Cinnd2 | 0.003951 | −4.28266 | 0.006775 | −3.49505 |
| If3ra1 | 0.011403 | −5.56161 | 0.029675 | −3.47974 |
| Gm6455 | 0.001394 | −2.33321 | 0.016851 | −3.45197 |
| Acyr2a | 0.003272 | −4.31328 | 0.004927 | −3.44986 |
| Alp1b1 | 0.002818 | −5.09177 | 0.004927 | −3.43851 |
| Tagap | 0.005249 | −2.93881 | 0.005537 | −3.42573 |
| AI451617 | 0.011115 | −3.67662 | 0.016851 | −3.42395 |
| Nrp1 | 0.008744 | −4.1097 | 0.01571 | −3.40887 |
| Gm10825 | 0.020543 | −3.5704 | 0.028211 | −3.40475 |
| Slc41a2 | 0.004144 | −4.86748 | 0.009405 | −3.40416 |
| Clso4a2 | 0.031962 | −4.34837 | 0.082522 | −3.39819 |
| II6st | 0.001506 | −3.68424 | 0.000899 | −3.39518 |
| Lmo2 | 0.027071 | −3.21103 | 0.030043 | −3.38896 |
| IIga1 | 0.010889 | −4.01025 | 0.019274 | −3.37381 |
| Ptactr2 | 0.017213 | −3.40384 | 0.021957 | −3.38738 |
| Lrrc16a | 0.006504 | −3.99184 | 0.011352 | −3.35025 |
| Lilrb3 | 0.018499 | −4.38833 | 0.032453 | −3.34939 |
| Zc3h12c | 0.007387 | −3.88449 | 0.012548 | −3.3432 |
| Slc8a1 | 0.003272 | −3.29089 | 0.004015 | −3.3374 |
| Grr9766 | 0.008427 | −3.20472 | 0.009969 | −3.33057 |
| Opn3 | 0.006934 | −3.63795 | 0.010434 | −3.32322 |
| Myn9a | 0.062801 | −3.29837 | 0.078049 | −3.31645 |
| Lzlfl1 | 0.042479 | −3.86222 | 0.071952 | −3.30559 |
| Xkra | 0.014696 | −4.38277 | 0.029777 | −3.30067 |
| Tgfbl | 0.022388 | −2.88061 | 0.021864 | −3.27199 |
| Phf11 | 0.028594 | −2.97028 | 0.028959 | −3.27024 |
| Tir1 | 0.017407 | −3.4984 | 0.024549 | −3.26533 |
| Stom | 0.015615 | −3.496 | 0.022468 | −3.26457 |
| Pak1 | 0.041473 | −3.29842 | 0.053075 | −3.26436 |
| Oec12a | 0.003273 | −3.63131 | 0.004828 | −3.2635 |
| Fmnl2 | 0.003273 | −4.05057 | 0.005606 | −3.24265 |
| Sgpl1 | 0.003971 | −3.67188 | 0.006414 | −3.22554 |
| Cusp22 | 0.004998 | −3.91828 | 0.009209 | −3.21719 |
| Bst1 | 0.011846 | −3.14712 | 0.014837 | −3.20312 |
| Runx2 | 0.003068 | −3.78331 | 0.004558 | −3.19681 |
| Myadm | 0.006488 | −3.90471 | 0.011998 | −3.19267 |
| Gm7909 | 0.002491 | −3.27171 | 0.002972 | −3.184 |
| Ccl9 | 0.010324 | −3.39987 | 0.015179 | −3.18384 |
| LOC825380 | 0.008381 | −3.45129 | 0.012733 | −3.16206 |
| Car2 | 0.077824 | −2.52461 | 0.058104 | −3.15914 |
| Ly6c2 | 0.008718 | −3.9723 | 0.017327 | −3.15768 |
| Fgl2 | 0.026519 | −3.36967 | 0.038202 | −3.13761 |
| Pbnb2 | 0.008528 | −3.5579 | 0.14305 | −3.13568 |
| Znb2 | 0.013542 | −3.50539 | 0.02159 | −3.13535 |
| Mlt4 | 0.017407 | −2.29071 | 0.010424 | −3.12985 |
| Mclp2 | 0.003273 | −3.83529 | 0.005608 | −3.11978 |
| Cds1 | 0.010631 | −3.65376 | 0.019068 | −3.1136 |
| Grn6455 | 0.017048 | −2.01477 | 0.007259 | −3.1117 |
| Ly6d | 0.001506 | −3.10426 | 0.001504 | −3.10515 |
| Bcl6 | 0.010464 | −3.22686 | 0.014766 | −3.09441 |
| Sirpa | 0.033089 | −3.96266 | 0.066691 | −3.09365 |
| BC013712 | 0.003273 | −3.3181 | 0.004512 | −3.09339 |
| Nalp5 | 0.013421 | −4.03539 | 0.027197 | −3.09068 |
| Samhd1 | 0.016217 | −3.18557 | 0.022193 | −3.0492 |
| Myd9a | 0.019857 | −3.2777 | 0.028838 | −3.04452 |
| Bclp2 | 0.046465 | −2.90523 | 0.054216 | −3.01248 |
| Ctnna1 | 0.003272 | −3.45483 | 0.004719 | −3.00523 |
| Hck | 0.002818 | −3.38896 | 0.004138 | −3.0042 |
| Dock1 | 0.004519 | −3.45042 | 0.007899 | −3.0038 |
| Hp | 0.021168 | −3.25394 | 0.03132 | −3.00334 |
| Rgs10 | 0.003889 | −3.58927 | 0.006634 | −2.99495 |
| Tc14 | 0.003272 | −3.55898 | 0.004831 | −2.99478 |
| Myo9a | 0.008073 | −3.24377 | 0.012106 | −2.98683 |
| Sp140 | 0.026342 | −3.85331 | 0.054713 | −2.97868 |
| Pickhm3 | 0.002044 | −3.77711 | 0.002972 | −2.96878 |
| Pira3 | 0.008877 | −4.11796 | 0.021065 | −2.96824 |
| Fam105a | 0.004721 | −3.10383 | 0.007037 | −2.95883 |
| 6330407A03Rik | 0.065182 | −2.96797 | 0.071043 | −2.94431 |
| Lyn | 0.002638 | −3.10355 | 0.002972 | −2.93382 |
| Ccdc88a | 0.014287 | −3.48546 | 0.2506 | −2.93298 |
| Fdg4 | 0.012079 | −3.33301 | 0.20596 | −2.9283 |
| EG685955 | 0.058056 | −2.47279 | 0.048638 | −2.91598 |
| Pras1 | 0.028294 | −4.08619 | 0.069844 | −2.91514 |
| B4galt6 | 0.022185 | −2.72175 | 0.023963 | −2.91458 |
| Zfp36 | 0.003579 | −2.95894 | 0.004745 | −2.91152 |
| A530040E14Rik | 0.00481 | −3.5706 | 0.00917 | −2.90236 |
| Myo1l | 0.011115 | −3.13927 | 0.017172 | −2.89816 |
| AY512938 | 0.017788 | −3.22302 | 0.027365 | −2.89768 |
| Cd52 | 0.024431 | −3.24056 | 0.039072 | −2.89981 |
| Tmem50b | 0.007785 | −2.83907 | 0.000572 | −2.88194 |
| Cox6a2 | 0.004633 | −3.03853 | 0.007037 | −2.87246 |
| Rab32 | 0.018322 | −3.12572 | 0.027051 | −2.87243 |
| Cc2d2a | 0.154998 | −1.51888 | 0.022881 | −2.8705 |
| A530040E14Rik | 0.033637 | −3.6478 | 0.075763 | −2.86734 |
| Grn | 0.003949 | −3.28363 | 0.006414 | −2.84639 |
| Cd34 | 0.024196 | −2.34643 | 0.019619 | −2.83926 |
| Vwa5a | 0.003966 | −3.00488 | 0.00561 | −2.8369 |
| Adora3 | 0.005408 | −3.06458 | 0.008853 | −2.83289 |
| Mef2a | 0.003272 | −2.8434 | 0.004015 | −2.82994 |
| Pip4k2a | 0.004144 | −2.75322 | 0.00581 | −2.80284 |
| Trt2 | 0.002818 | −3.13212 | 0.004015 | −2.80145 |
| Syna2 | 0.00318 | −4.7624 | 0.007509 | −2.79447 |
| Gpr183 | 0.012838 | −3.16694 | 0.020617 | −2.78912 |
| Ccnd1 | 0.011431 | −2.98879 | 0.017599 | −2.78849 |
| A530023O14Rik | 0.004144 | −2.66025 | 0.005327 | −2.78598 |
| Anid5b | 0.003798 | −3.00785 | 0.005537 | −2.7735 |
| Tmam224 | 0.008695 | −3.1391 | 0.014711 | −2.76845 |
| Havor2 | 0.007573 | −3.115 | 0.012632 | −2.76215 |
| Gm2a | 0.004878 | −2.48507 | 0.005427 | −2.74955 |
| Pik3ap1 | 0.005255 | −2.98508 | 0.006553 | −2.74603 |
| Pik3c2a | 0.005288 | −2.88522 | 0.008018 | −2.74014 |
| Unc93b1 | 0.003272 | −2.78919 | 0.004202 | −2.73784 |
| Zc3h12c | 0.005659 | −3.11446 | 0.009869 | −2.73350 |
| Asah1 | 0.010589 | −2.91192 | 0.015783 | −2.73282 |
| Gcnt2 | 0.007313 | −2.50357 | 0.008087 | −2.73227 |
| Arhgef12 | 0.007511 | −3.36202 | 0.015283 | −2.71628 |
| A030009H04Rik | 0.002348 | −2.90581 | 0.002972 | −2.71553 |
| Tceal8 | 0.012516 | −2.71384 | 0.016359 | −2.71046 |
| Csl3 | 0.010432 | −2.89285 | 0.01571 | −2.70141 |
| Lsp1 | 0.005063 | −2.77221 | 0.007264 | −2.69996 |
| Mobkl2b | 0.052746 | −3.00921 | 0.085365 | −2.69286 |
| A539040E14Rik | 0.007591 | −3.89434 | 0.000663 | −2.68538 |
| Blnk | 0.00947 | −4.09852 | 0.027012 | −2.69514 |
| Ela2 | 0.034814 | −2.71218 | 0.044142 | −2.68383 |
| Ggh | 0.016991 | −2.91635 | 0.028711 | −2.6838 |
| Itgh2 | 0.004898 | −2.7059 | 0.006803 | −2.67768 |
| Sgrns1 | 0.018991 | −4.49078 | 0.020784 | −2.67271 |
| Kirb1f | 0.004144 | −3.4359 | 0.006863 | −2.66766 |
| Rgl1 | 0.003793 | −3.15257 | 0.006343 | −2.66586 |
| Sp140 | 0.008695 | −3.3058 | 0.01823 | −2.65738 |
| LySc1 | 0.02483 | −3.45083 | 0.055021 | −2.85431 |
| Dnajb14 | 0.007313 | −3.49144 | 0.017172 | −2.64783 |
| Dnase1l1 | 0.017407 | −3.05358 | 0.29637 | −2.64321 |
| Ap1s2 | 0.022368 | −2.98021 | 0.037326 | −2.64315 |
| Ifgam | 0.053551 | −2.65347 | 0.068644 | −2.64008 |
| Eepd1 | 0.006015 | −2.95588 | 0.010048 | −2.62803 |
| Fam55a | 0.028005 | −3.14485 | 0.052952 | −2.62564 |
| Gm9733 | 0.043995 | −4.43351 | 0.147784 | −2.62553 |
| Skap2 | 0.005648 | −2.77114 | 0.008883 | −2.61226 |
| Gsdrnd | 0.020507 | −2.75432 | 0.02936 | −2.60355 |
| Cfp | 0.012079 | −2.86736 | 0.019709 | −2.60201 |
| Pln | 0.052658 | −2.25028 | 0.044502 | −2.60068 |
| Tmem176b | 0.008075 | −2.59973 | 0.010424 | −2.58074 |
| Hgf | 0.004128 | −2.02709 | 0.021664 | −2.57983 |
| Ppm18 | 0.020359 | −2.71912 | 0.028838 | −2.57715 |
| Tram3 | 0.035118 | −2.63778 | 0.046868 | −2.57826 |
| Camsap1l1 | 0.003273 | −2.69535 | 0.004685 | −2.56938 |
| Abcb1b | 0.018994 | −2.5155 | 0.022414 | −2.5846 |
| Zlp677 | 0.043542 | −2.68382 | 0.082198 | −2.56236 |
| Nrp3 | 0.007389 | −2.50125 | 0.009098 | −2.58223 |
| Parp6 | 0.002146 | −2.67684 | 0.002889 | −2.56192 |
| Slc46a3 | 0.035496 | −2.91315 | 0.061829 | −2.54691 |
| Lpcat2 | 0.037338 | −2.32991 | 0.036538 | −2.54537 |
| Camk1d | 0.005668 | −2.91954 | 0.010244 | −2.53895 |
| Txndc16 | 0.011506 | −3.06007 | 0.022361 | −2.53889 |
| 3830403N18Rik | 0.154984 | −2.85118 | 0.201978 | −2.53822 |
| Dennd5a | 0.008115 | −2.51281 | 0.010009 | −2.53272 |
| Sh2d1b1 | 0.064024 | −2.81547 | 0.101747 | −2.53172 |
| Satbp1 | 0.00461 | −3.77882 | 0.013712 | −2.5238 |
| Nek6 | 0.006468 | −2.46833 | 0.008512 | −2.51941 |
| Ppp2r5c | 0.061779 | −2.91456 | 0.108266 | −2.51186 |
| Myo9a | 0.110573 | −2.64868 | 0.150197 | −2.51017 |
| Clec4a1 | 0.06775 | −3.36155 | 0.152763 | −2.5081 |
| Itr2 | 0.244902 | −2.00491 | 0.174072 | −2.50633 |
| Ifl2712a | 0.017407 | −3.13841 | 0.036418 | −2.50223 |
| Znb2 | 0.004757 | −3.03141 | 0.009405 | −2.49526 |
| Snx9 | 0.004144 | −2.58277 | 0.006343 | −2.4769 |
| Hst1b2bc | 0.031187 | −3.15858 | 0.089659 | −2.47272 |
| Lpca1 | 0.006685 | −2.50679 | 0.009023 | −2.48868 |
| Lipa | 0.022099 | −2.55063 | 0.0301 | −2.46375 |
| Grand3 | 0.012827 | −2.37201 | 0.014934 | −2.46355 |
| Slc44a1 | 0.006488 | −3.81219 | 0.021065 | −2.46223 |
| Pgcp | 0.017048 | −2.6951 | 0.026587 | −2.46200 |
| Rtn1 | 0.02295 | −2.99996 | 0.046794 | −2.45617 |
| Gm14005 | 0.04514 | −2.28062 | 0.046535 | −2.45606 |
| Net1 | 0.003579 | −2.68986 | 0.005361 | −2.44849 |
| Pja1 | 0.003273 | −2.48028 | 0.004512 | −2.44583 |
| 5033414K04Rik | 0.006137 | −2.9068 | 0.011998 | −2.44006 |
| Tnni2 | 0.009564 | −2.58137 | 0.014627 | −2.43993 |
| Ppp2r5c | 0.01387 | −2.93747 | 0.026773 | −2.43783 |
| Itgae | 0.05613 | −2.2474 | 0.056409 | −2.43415 |
| Atp7a | 0.003966 | −2.77382 | 0.006775 | −2.425 |
| Igsl6 | 0.021997 | −2.72272 | 0.037235 | −2.4258 |
| Depdc7 | 0.059216 | −2.76294 | 0.101548 | −2.42324 |
| Myo9a | 0.021997 | −2.70507 | 0.037401 | −2.40888 |
| Tiparp | 0.00912 | −2.57423 | 0.014305 | −2.40506 |
| Cd53 | 0.01089 | −2.43294 | 0.014711 | −2.39885 |
| Spp1 | 0.009483 | −2.33086 | 0.04573 | −2.39663 |
| Alp2b4 | 0.019125 | −2.46361 | 0.028031 | −2.39588 |
| Ctnnd1 | 0.009821 | −2.16542 | 0.009405 | −2.39296 |
| Plck | 0.016683 | −2.87307 | 0.027401 | −2.38224 |
| Chi3I3 | 0.123789 | −2.86072 | 0.218586 | −2.38477 |
| Myo9a | 0.189365 | −2.32603 | 0.214975 | −2.36398 |
| Chrs7 | 0.00872 | −3.15425 | 0.021664 | −2.36089 |
| Myo9a | 0.022796 | −2.65759 | 0.039286 | −2.35855 |
| Tubb2a | 0.011115 | −2.55191 | 0.01823 | −2.35428 |
| Inpp4a | 0.009412 | −2.86138 | 0.019619 | −2.35269 |
| Hist2h2ba | 0.005859 | −3.05378 | 0.014305 | −2.34434 |
| Lrrtm2 | 0.044764 | −2.55489 | 0.071807 | −2.34133 |
| Rgs2 | 0.011115 | −2.39088 | 0.015244 | −2.34067 |
| Snx24 | 0.023133 | −2.57233 | 0.037796 | −2.33393 |
| Tubgcp5 | 0.006877 | −3.1233 | 0.022321 | −2.33153 |
| Cask | 0.010322 | −2.88335 | 0.018981 | −2.32927 |
| 4930429K28Rik | 0.01913 | −2.42247 | 0.027051 | −2.31823 |
| Hdac9 | 0.00533 | −3.26373 | 0.015434 | −2.31581 |
| Hepacam2 | 0.047563 | −3.30972 | 0.134464 | −2.31478 |
| Ctbs | 0.027071 | −2.54119 | 0.043739 | −2.31153 |
| Alg4c | 0.045482 | −2.18179 | 0.048371 | −2.31084 |
| Fam174a | 0.003273 | −2.63053 | 0.005327 | −2.30382 |
| Gm10708 | 0.043667 | −2.28306 | 0.054002 | −2.30173 |
| Abog3 | 0.05938 | −1.84622 | 0.038395 | −2.29012 |
| Cleo4b2 | 0.022196 | −2.77147 | 0.046243 | −2.28893 |
| A930001N09Rik | 0.013226 | −2.3334 | 0.018274 | −2.2868 |
| Skil | 0.011851 | −2.52339 | 0.020025 | −2.28675 |
| Tips | 0.017895 | −1.88609 | 0.124154 | −2.28297 |
| Cd22 | 0.049466 | −2.24048 | 0.060045 | −2.27899 |
| Raph1 | 0.006427 | −3.56151 | 0.028773 | −2.27642 |
| 9630617O03Rik | 0.019587 | −2.15544 | 0.021831 | −2.27468 |
| Stx7 | 0.002816 | −2.55486 | 0.004238 | −2.27018 |
| Gap1 | 0.021997 | −2.63517 | 0.041632 | −2.26586 |
| Mtm1 | 0.006345 | −2.45248 | 0.010048 | −2.25447 |
| Ef2c4 | 0.005408 | −2.64977 | 0.010452 | −2.26326 |
| Tbc1d9 | 0.024007 | −2.20907 | 0.027921 | −2.25719 |
| Ly96 | 0.060142 | −2.50422 | 0.099214 | −2.25287 |
| Itpr1 | 0.003415 | −2.50152 | 0.005361 | −2.25251 |
| Lztfl1 | 0.011587 | −2.42049 | 0.018594 | −2.25239 |
| Ap1s3 | 0.005473 | −2.27241 | 0.007721 | −2.25188 |
| B3gnt5 | 0.043904 | −2.19284 | 0.051774 | −2.24436 |
| Tifab | 0.058976 | −2.6749 | 0.115897 | −2.2391 |
| Bcl2a1c | 0.014376 | −2.3524 | 0.021247 | −2.23818 |
| Scx11 | 0.008115 | −2.51909 | 0.014711 | −2.23397 |
| Prtn3 | 0.027318 | −2.31057 | 0.037857 | −2.23028 |
| 3110043O21Rik | 0.005648 | −2.25119 | 0.007948 | −2.23019 |
| Cd44 | 0.012078 | −2.13021 | 0.013987 | −2.22992 |
| Ero1lb | 0.011057 | −2.58214 | 0.02115 | −2.22875 |
| Met | 0.02249 | −2.46002 | 0.037514 | −2.22869 |
| Meta1 | 0.010601 | −1.97629 | 0.00938 | −2.22877 |
| PRK1 | 0.027728 | −2.89698 | 0.058908 | −2.22148 |
| Strpb1 | 0.049807 | −2.93038 | 0.124209 | −2.21679 |
| A530032D15Rik | 0.036256 | −2.99687 | 0.096674 | −2.21603 |
| Zfp229 | 0.075248 | −2.33866 | 0.107985 | −2.21498 |
| Myo9a | 0.005913 | −2.45424 | 0.010848 | −2.2148 |
| B16rap | 0.085214 | −1.99604 | 0.076877 | −2.21375 |
| Trim30 | 0.013421 | −2.29105 | 0.01928 | −2.20982 |
| Rufy1 | 0.008535 | −2.41096 | 0.013282 | −2.20682 |
| Ncf1 | 0.081983 | −2.37103 | 0.049469 | −2.20684 |
| Tcf712 | 0.011846 | −1.91294 | 0.009583 | −2.18763 |
| Cd388lf | 0.004144 | −1.89568 | 0.004585 | −2.19725 |
| Xis1 | 0.908468 | −1.94981 | 0.897825 | −2.19583 |
| Ly6a | 0.043621 | −2.68801 | 0.086508 | −2.19326 |
| Cita | 0.023059 | −2.38656 | 0.037106 | −2.18307 |
| Lyst | 0.004633 | −2.48203 | 0.008816 | −2.19231 |
| Ctsb | 0.003783 | −2.51918 | 0.008343 | −2.18175 |
| Cx3or1 | 0.0174 | 2.25579 | 0.023436 | −2.19122 |
| Ccr9 | 0.039479 | −3.4137 | 0.137524 | −2.19115 |
| Glpr1 | 0.021997 | −2.22748 | 0.028838 | −2.19048 |
| Emb | 0.011772 | −2.2619 | 0.017829 | −2.18983 |
| Klf6 | 0.003889 | −2.4037 | 0.006258 | −2.1885 |
| Nrc4 | 0.024314 | −2.52062 | 0.045557 | −2.18521 |
| Sgk3 | 0.007833 | −2.32786 | 0.010755 | −2.18504 |
| Lair1 | 0.014287 | −2.36315 | 0.021247 | −2.18473 |
| Co8a | 0.026614 | −2.039 | 0.028158 | −2.18454 |
| Fam69a | 0.008475 | −2.49086 | 0.017869 | −2.18309 |
| Mtsd6 | 0.010883 | −2.35778 | 0.01773 | −2.1813 |
| Ifnar2 | 0.00459 | −2.31182 | 0.007264 | −2.1733 |
| II1b | 0.047125 | −3.05025 | 0.13887 | −2.17271 |
| Ifl30 | 0.024431 | −3.22531 | 0.082254 | −2.1725 |
| Tmeff1 | 0.02137 | −3.48083 | 0.039533 | −2.18843 |
| Gm10833 | 0.040654 | −1.81127 | 0.027051 | −2.16872 |
| H2-Oa | 0.022514 | −2.24172 | 0.03147 | −2.1618 |
| Cedc90b | 0.017393 | −2.13781 | 0.021421 | −2.16071 |
| Pik2 | 0.010085 | −2.49758 | 0.018618 | −2.16011 |
| Tmam71 | 0.017407 | −2.13219 | 0.021416 | −2.15748 |
| Epb4.113 | 0.040574 | −1.7106 | 0.021772 | −2.15587 |
| Bcl11a | 0.070838 | −2.10622 | 0.082558 | −2.154 |
| B3gnt3 | 0.443073 | −1.22087 | 0.043872 | −2.15397 |
| Capn2 | 0.005154 | −2.72151 | 0.012106 | −2.15209 |
| Rga18 | 0.074895 | −2.16818 | 0.09473 | −2.14997 |
| 8330442E10Rik | 0.015931 | −2.29422 | 0.023267 | −2.14796 |
| Slpl1 | 0.066473 | −2.08323 | 0.084902 | −2.14343 |
| II18 | 0.013535 | −2.58102 | 0.02759 | −2.14358 |
| Serpina12 | 0.182707 | −1.53034 | 0.063726 | −2.14222 |
| 9230105E10Rik | 0.023829 | −2.06682 | 0.024144 | −2.13623 |
| Hoxa5 | 0.005258 | −2.1122 | 0.007037 | −2.13816 |
| Gag2 | 0.002818 | −2.48455 | 0.004512 | −2.13337 |
| Cysltrl | 0.038423 | −2.59769 | 0.086043 | −2.12983 |
| St8sia4 | 0.004334 | −2.51892 | 0.009 | −2.12781 |
| Ptprj | 0.003422 | −2.49266 | 0.006258 | −2.12524 |
| Ssh2 | 0.300797 | −1.88875 | 0.2882 | −2.12112 |
| 1700010114Rik | 0.024431 | −2.09074 | 0.028864 | −2.12096 |
| Usp12 | 0.019242 | −2.09373 | 0.023438 | −2.11902 |
| Tlr2 | 0.021708 | −2.6856 | 0.053127 | −2.11662 |
| Cacna1e | 0.021283 | −2.45825 | 0.041632 | −2.11518 |
| Ms4a6d | 0.023202 | −2.52453 | 0.046522 | −2.11423 |
| Slc37a3 | 0.152733 | −1.83785 | 0.120628 | −2.11369 |
| Emr1 | 0.057580 | −2.83011 | 0.148945 | −2.1133 |
| Nco81 | 0.003793 | −2.58723 | 0.007269 | −2.11289 |
| Kctd14 | 0.014826 | −2.3192 | 0.024357 | −2.11003 |
| #203 | 0.011794 | −2.34477 | 0.020816 | −2.1075 |
| Alpk1 | 0.019857 | −2.4115 | 0.037298 | −2.10436 |
| Mx2 | 0.016976 | −2.88463 | 0.040958 | −2.1033 |
| Gm6455 | 0.375376 | −2.38353 | 0.109488 | −2.08854 |
| Clec5a | 0.060611 | −2.16285 | 0.083815 | −2.09812 |
| C668 | 0.010881 | −2.31654 | 0.019068 | −2.09712 |
| Ex2a | 0.013963 | −2.42595 | 0.026866 | −2.09326 |
| DlErtd622a | 0.005249 | −2.58387 | 0.008023 | −2.08211 |
| Myc88 | 0.024007 | −2.50056 | 0.050885 | −2.08875 |
| Rbm47 | 0.013298 | −2.33835 | 0.023123 | −2.08807 |
| Nostrin | 0.021815 | −2.80364 | 0.062387 | −2.08626 |
| Hmgn3 | 0.016421 | −2.74047 | 0.043124 | −2.08298 |
| Nsmal | 0.007211 | −2.38584 | 0.014305 | −2.07672 |
| Hexa | 0.011826 | −2.34523 | 0.021664 | −2.07535 |
| #204 | 0.062104 | −2.504179 | 0.130837 | −2.06589 |
| Srgap3 | 0.004023 | −2.70757 | 0.009669 | −2.06559 |
| Lg8ls3 | 0.015466 | −2.5131 | 0.033485 | −2.08488 |
| Ms4a4b | 0.024123 | −2.08886 | 0.031359 | −2.05829 |
| Prop | 0.026215 | −2.1637 | 0.038861 | −2.05037 |
| Chd7 | 0.2629 | −2.17545 | 0.334791 | −2.05027 |
| K81c | 0.027071 | −2.25257 | 0.045412 | −2.05005 |
| Clec4a3 | 0.060762 | −3.40871 | 0.200588 | −2.04828 |
| Gm1986 | 0.014886 | −2.34608 | 0.027197 | −2.04804 |
| Sk364 | 0.032161 | −2.41381 | 0.086574 | −2.048 |
| Slc9a2 | 0.056989 | −1.69922 | 0.035562 | −2.04158 |
| Tymbp | 0.038955 | −2.07164 | 0.053127 | −2.03537 |
| Ssbp2 | 0.017549 | −2.0847 | 0.023493 | −2.03428 |
| Thbs1 | 0.12524 | −1.74414 | 0.090282 | −2.03353 |
| F13a1 | 0.121799 | −2.48127 | 0.233422 | −2.03318 |
| Lapml1 | 0.055438 | −2.3881 | 0.111057 | −2.0314 |
| Slrpb1 | 0.057325 | −3.5485 | 0.23801 | −2.03636 |
| Malt1 | 0.004085 | −2.23 | 0.006803 | −2.0297 |
| Tpx152 | 0.008073 | −2.06385 | 0.01078 | −2.02988 |
| Cybasc3 | 0.013542 | −2.21783 | 0.022381 | −2.02835 |
| Otud1 | 0.010123 | −2.4137 | 0.021664 | −2.02882 |
| Bex6 | 0.024414 | −2.12771 | 0.038045 | −2.0278 |
| Scp2 | 0.01931 | −1.94896 | 0.021884 | −2.02724 |
| Arhgap17 | 0.003795 | −2.27149 | 0.008343 | −2.02578 |
| Slc8a1 | 0.017788 | −2.23679 | 0.02966 | −2.02576 |
| Dap | 0.003972 | −2.26617 | 0.007037 | −2.02274 |
| Fcgt | 0.018091 | −2.21658 | 0.02988 | −2.01807 |
| Amica1 | 0.011826 | −2.46832 | 0.028767 | −2.01773 |
| Ptalr | 0.013181 | −2.08575 | 0.019086 | −2.01713 |
| Abcg2 | 0.019879 | −1.997 | 0.023883 | −2.01678 |
| Ext1 | 0.011019 | −2.07243 | 0.015571 | −2.01846 |
| Cxcr6 | 0.053286 | −1.97607 | 0.063195 | −2.01568 |
| Stambpl1 | 0.009739 | −2.73376 | 0.026461 | −2.00776 |
| Gm16485 | 0.355729 | −1.28935 | 0.065551 | −2.00753 |
| Nfam1 | 0.018599 | −2.20381 | 0.030848 | −2.00413 |
| Rix3 | 0.013148 | −1.9598 | 0.01571 | −2.00087 |
| Gpr155 | 0.00836 | −2.93233 | 0.022361 | −2.00015 |

TABLE 3
Genes greater than 2 fold in Tcf1-expressing Thy1 + CD25+
cells compared to LMPP

| | Tcf1-T #1 | Tcf1-T #2 | Control T |
|---|---|---|---|
| #2ra | 85.283 | 69.085 | 107.971 |
| Vcan | 32.204 | 34.1385 | 7.33422 |
| H19 | 58.6931 | 36.2334 | 46.354 |
| Rhoj | 20.4504 | 24.5746 | 1.47782 |
| Rlp4 | 19.983 | 15.7754 | 10.509 |
| Thy1 | 19.1157 | 21.1487 | 28.9979 |
| Emr1 | 18.4886 | 23.2033 | −1.30811 |
| Rasgrp1 | 17.289 | 21.0993 | 35.2508 |
| 5838411N86Rik | 17.0945 | 14.8446 | 8.68864 |
| Oax2 | 15.9548 | 10.1037 | 2.81029 |
| F13a1 | 14.9875 | 13.8541 | −1.13182 |
| Scd1 | 13.3469 | 13.6089 | 18.5024 |
| Fam188b | 12.4249 | 14.8434 | 22.8443 |
| Timp3 | 11.8773 | 15.4166 | 12.8501 |
| Psg17 | 11.8153 | 17.5169 | 24.1252 |
| Akr1c12 | 11.1967 | 16.7621 | 21.473 |
| Ms4a6b | 10.9599 | 11.3236 | 12.1852 |
| Nalp3 | 10.4559 | 8.8626 | 10.3127 |
| Ms4a4v | 10.118 | 11.011 | −1.34616 |
| Aro | 10.089 | 8.44785 | 4.82513 |
| Papss2 | 8.98028 | 18.6366 | 2.25286 |
| Tcrp-V3 | 9.85008 | 9.32138 | 8.62168 |
| Txk | 8.72213 | 12.9856 | 25.6943 |
| Oasi2 | 9.86444 | 7.8505 | 5.1575 |
| Thbs1 | 8.85777 | 8.554 | 5.83157 |
| Enah | 8.75945 | 10.0197 | 8.21857 |
| Tgfb1 | 8.18825 | 8.80004 | −1.42278 |
| ltk | 8.03243 | 8.88859 | 8.77184 |
| Ma4a8d | 8.0229 | 8.72282 | −1.90007 |
| Ddx58 | 7.74671 | 7.02718 | 8.30975 |
| Ccdc189b | 7.4994 | 10.3707 | 14.8351 |
| Sif85 | 7.27791 | 7.48454 | 4.17685 |
| #7r | 7.13921 | 8.35861 | 4.74067 |
| A638838E17Rik | 8.68874 | 8.19594 | 12.5983 |
| Fam40b | 6.55424 | 9.42944 | 15.3104 |
| Gprin3 | 6.48236 | 8.8229 | 8.9285 |
| Cysltr1 | 6.3872 | 7.27103 | 5.97907 |
| Nov | 6.27173 | 8.85576 | 2.0479 |
| Al807873 | 6.01243 | 8.82883 | 1.80888 |
| Stxbp6 | 6.93694 | 7.86734 | 7.77169 |
| Mmp6 | 5.80958 | 5.67498 | −5.47815 |
| Ceacam15 | 5.85764 | 10.7496 | −1.00674 |
| Trat1 | 5.79019 | 7.18032 | 45.4621 |
| Cleo483 | 5.77812 | 8.04737 | −1.15204 |
| EG634650 | 5.7681 | 7.19231 | 15.035 |
| Mpa2f | 5.70952 | 8.26539 | 12.9143 |
| AB124811 | 5.70889 | 7.03538 | 6.17523 |
| Var11 | 5.70762 | 8.85018 | 11.3038 |
| A130014H13Rik | 5.53268 | 3.79705 | 15.151 |
| Castg | 5.5151 | 4.54253 | 1.04017 |
| Usp18 | 5.47098 | 4.86466 | 1.89096 |
| Pda3b | 5.40883 | 8.11325 | 4.54785 |
| Mgl2 | 5.3162 | 4.80712 | −3.38478 |
| Mgst2 | 5.28637 | 4.77588 | 5.88711 |
| Prkc8 | 5.27485 | 6.78225 | 11.8579 |
| Ms484b | 5.26066 | 6.431 | 3.17501 |
| Gala3 | 5.18815 | 6.5222 | 14.275 |
| Clec481 | 5.12594 | 10.8144 | −1.2246 |
| Oas1b | 5.08925 | 4.26269 | 3.81015 |
| Akr1c13 | 5.06322 | 6.33518 | 8.98927 |
| Ifit1 | 5.04806 | 3.78882 | 1.78018 |
| Kblbd11 | 4.88941 | 6.09583 | 1.9216 |
| Ncam2 | 4.81527 | 7.48494 | 1.22177 |
| Pbdc2 | 4.71542 | 6.03838 | 7.10576 |
| Cpa3 | 4.66979 | 5.84662 | 8.22892 |
| Dnx58 | 4.6221 | 4.428846 | 5.29429 |
| Fam20a | 4.60045 | 6.78821 | 45.7918 |
| Pd22a | 4.53628 | 5.81654 | 3.71881 |
| Ccr4 | 4.45242 | 6.3847 | 12.8645 |
| Nck2 | 4.41682 | 5.72714 | 12.637 |
| Pamp12 | 4.41 | 4.18855 | 2.97508 |
| Nck2 | 4.39988 | 4.79517 | 8.8353 |
| Gm13928 | 4.38441 | 4.56013 | 4.43226 |
| Gm13928 | 4.38441 | 4.58013 | 4.43228 |
| Xaf1 | 4.38293 | 3.75306 | 2.88888 |
| Pdcd1 | 4.3563 | 4.85336 | 1.84898 |
| P2ry10 | 4.29736 | 5.5801 | 9.89975 |
| Tgtp | 4.24791 | 4.10055 | 7.70997 |
| Xkrx | 4.23521 | 6.84851 | 1.87873 |
| Sla2 | 4.16 | 5.33141 | 4.87248 |
| Pyrin1 | 4.14375 | 5.40156 | 2.09191 |
| Rasgrp3 | 4.11607 | 5.63549 | −1.48819 |
| lrfy | 4.1122 | 3.81986 | 2.57636 |
| Gxln1 | 4.04883 | 3.68669 | 2.03201 |
| #2711 | 4.02135 | 4.29508 | 2.12873 |
| Tr8 | 4.01948 | 4.08954 | −1.09962 |
| E83007K03Rik | 4.01243 | 4.70814 | 2.03656 |
| Iff44 | 4.99436 | 5.28832 | 1.88408 |
| Ms4a6c | 4.98165 | 3.46087 | −3.4711 |
| I830127L87Rik | 4.96439 | 4.2802 | −5.01406 |
| Igf2f | 3.95268 | 4.54002 | 3.26247 |
| Bc116 | 3.94658 | 4.51171 | 7.26869 |
| Stamf1 | 3.94361 | 5.72018 | 3.68777 |
| Tnfrsf8 | 3.92113 | 5.93165 | 2.38741 |
| Tgtp | 3.85268 | 3.71398 | 7.12875 |
| Lyz2 | 3.81845 | 6.09709 | −3.87077 |
| H18 | 3.79615 | 6.03294 | 6.5614 |
| Tgfbr3 | 3.7951 | 4.01881 | 4.43878 |
| Mov10 | 3.76182 | 3.08481 | 3.57263 |
| Epstl1 | 3.72237 | 4.45143 | 6.12516 |
| Bgn | 3.71557 | 8.10584 | 1.66414 |
| Grap2 | 3.60224 | 4.1206 | 7.7657 |
| Clla4 | 3.59217 | 4.71656 | 33.6475 |
| Ppargc1a | 3.57943 | 5.21428 | 1.19033 |
| II17rb | 3.57519 | 4.0946 | 15.4522 |
| Tcn2 | 3.58485 | 3.78647 | 2.19513 |
| Mfsd2 | 3.58058 | 3.95492 | 6.47424 |
| Socs2 | 3.55823 | 4.06481 | 2.62153 |
| Gamb | 3.54473 | 5.80707 | 5.24799 |
| Ly6a | 3.49779 | 2.95369 | 2.35028 |
| Gbp4 | 3.48065 | 4.38875 | 10.0336 |
| Cct22 | 3.47788 | 6.83311 | −3.08197 |
| Gpr114 | 3.47442 | 4.36041 | 8.46593 |
| Els1 | 3.47434 | 4.27679 | 10.2491 |
| Trim30 | 3.47328 | 3.04261 | 2.36157 |
| Sirpb1 | 3.46106 | 4.85558 | −1.08533 |
| Gp49a | 3.38967 | 4.18117 | 2.78546 |
| Aclr3b | 3.33786 | 5.28524 | 9.94183 |
| Ccl5 | 3.33718 | 5.65498 | 1.14175 |
| Ap1s2 | 3.30202 | 4.2101 | −1.74432 |
| Rnf213 | 3.29799 | 2.71438 | 1.52882 |
| Pmp | 3.26849 | 4.40002 | 1.89597 |
| Saa3 | 3.28098 | 7.01476 | −1.42893 |
| Igf2 | 3.25889 | 5.38984 | 14.5674 |
| Samd9l | 3.24364 | 3.32221 | 1.09309 |
| Cd163I1 | 3.24084 | 3.28879 | 13.255 |
| Isg20 | 3.20824 | 3.63903 | 3.56453 |
| Cxcr5 | 3.18765 | 5.42289 | 14.1785 |
| Map3k8 | 3.17208 | 3.86416 | 5.03393 |
| Sipa1l1 | 3.16333 | 3.45906 | 2.24457 |
| Ifi27t2a | 3.15138 | 2.92347 | 1.06244 |
| Ms4a4a | 3.1312 | 4.6821 | −1.22057 |
| Ifl204 | 3.11573 | 3.03883 | 6.70827 |
| Ffar2 | 3.11187 | 3.94009 | 1.17369 |
| Zfp760 | 3.10535 | 4.35887 | 1.54049 |
| Mgl1 | 3.0804 | 3.26787 | −1.98664 |
| Faon1 | 3.07048 | 6.3335 | −1.05035 |
| C1glnf1 | 3.06806 | 4.08069 | 8.67316 |
| Ddx60 | 3.00533 | 2.81648 | 1.23348 |
| Igf1 | 3.00293 | 5.05856 | −1.09199 |
| Alrn | 2.97875 | 3.0549 | 1.98509 |
| Slk39 | 2.97435 | 3.61323 | 5.80791 |
| Lirb4 | 2.97183 | 3.23498 | 2.32116 |
| Gm4951 | 2.96881 | 2.20468 | 2.06748 |
| Fbxo30 | 2.9811 | 2.87463 | 4.66243 |
| Gm10838 | 2.9528 | 2.76581 | 5.81497 |
| Plpn1 | 2.94113 | 3.25883 | 2.10102 |
| Fyb | 2.93241 | 3.5787 | 6.14440 |
| Gm6683 | 2.9301 | 4.00034 | 15.949 |
| Ppargc1a | 2.92024 | 4.33806 | 1.12951 |
| Fut8 | 2.91025 | 3.10799 | 3.0108 |
| Socs2 | 2.89024 | 3.34723 | 2.50197 |
| Rras2 | 2.86416 | 3.33484 | 6.58337 |
| Snora81 | 2.88469 | 2.21891 | 2.39015 |
| R2rb | 2.85424 | 4.08673 | 1.75382 |
| Lef1 | 2.8473 | 2.95988 | 12.6548 |
| II180 | 2.82519 | 3.54569 | 7.14108 |
| Rsad2 | 2.82318 | 2.32386 | 1.61626 |
| Gpnmb | 2.80933 | 4.98071 | 1.03801 |
| Abog1 | 2.8084 | 3.28497 | 3.74347 |
| Hp | 2.80199 | 3.22152 | −3.51563 |
| Kit | 2.7985 | 3.00236 | 3.27225 |
| AI451617 | 2.76435 | 3.08162 | 2.40068 |
| Tcf7 | 2.76786 | 3.47102 | 16.934 |
| Cd247 | 2.75498 | 3.81144 | 8.94638 |
| Sema7a | 2.75301 | 4.40564 | 8.09443 |
| Gas3 | 2.74795 | 2.65328 | 1.26882 |
| Tubb3 | 2.74722 | 3.56189 | 4.01315 |
| Rnu3b1 | 2.74883 | 2.26233 | 2.2555 |
| Pira2 | 2.73934 | 4.0392 | −2.35096 |
| Tmem106 | 2.73377 | 3.4381 | −1.16982 |
| Itgb5 | 2.72127 | 3.25222 | −2.24297 |
| Cacnb3 | 2.71432 | 5.20222 | 1.8061 |
| Tmem35 | 2.70166 | 2.61846 | 1.05481 |
| Parp14 | 2.6887 | 2.73201 | 2.72297 |
| Adamts9 | 2.67264 | 3.71014 | 13.4575 |
| Lob | 2.88598 | 2.83004 | 5.46781 |
| Gas1a | 2.6582 | 2.52359 | −1.05972 |
| Tmtc2 | 2.65428 | 4.29331 | 7.83087 |
| Fn1 | 2.64929 | 5.28611 | 1.07509 |
| 3110062M02Rik | 2.64285 | 2.88044 | 2.16974 |
| Ppt1 | 2.63909 | 2.84207 | 3.36621 |
| Ar | 2.63865 | 4.66322 | 3.20608 |
| Pld4 | 2.63526 | 2.07256 | 7.88576 |
| Gcom1 | 2.63394 | 3.72412 | 6.0015 |
| Bsl1 | 2.62962 | 2.8517 | 1.81948 |
| Gas7 | 2.62897 | 3.27434 | 1.24217 |
| Tcra | 2.6242 | 3.38281 | 3.15337 |
| Tcra | 2.6242 | 3.38281 | 3.15337 |
| Slc22a23 | 2.62391 | 3.74705 | 2.56557 |
| Herc5 | 2.61287 | 2.75003 | 2.13316 |
| Rab44 | 2.61058 | 2.39395 | 1.54914 |
| Prkch | 2.60943 | 3.12968 | 6.41559 |
| 9630013D21Rik | 2.60389 | 4.21738 | 1.99186 |
| Gm4955 | 2.60528 | 2.51047 | 1.8414 |
| Aqp11 | 2.58828 | 3.11332 | 9.35194 |
| Pln2g7 | 2.58404 | 4.15871 | −1.16994 |
| Salp | 2.58041 | 2.84628 | −1.01285 |
| Cd2 | 2.57458 | 3.20197 | 5.49688 |
| Nrp2 | 2.5712 | 3.40144 | −1.0752 |
| 7120432I05Rik | 2.56355 | 2.89074 | 1.0967 |
| Iflt3 | 2.58258 | 2.39942 | 1.32021 |
| Lirb3 | 2.55935 | 2.09303 | −1.33757 |
| Lgals3bp | 2.5498 | 2.3828 | 1.56294 |
| Tnfrsf18 | 2.54568 | 4.73689 | 3.81427 |
| Offr524 | 2.53591 | 2.63858 | 5.35004 |
| Ahcyl2 | 2.52784 | 2.98385 | 4.08997 |
| Oam | 2.51484 | 2.5987 | 2.71027 |
| Gldc | 2.51126 | 3.97302 | 1.32391 |
| Sytl2 | 2.49191 | 3.47124 | 6.99711 |
| Tmam178 | 2.48354 | 2.80547 | −2.78067 |
| Flrt3 | 2.48018 | 2.63212 | −1.22686 |
| Tcm | 2.47589 | 2.93211 | 2.46402 |
| Fndc1 | 2.47204 | 3.45732 | 1.24434 |
| Dgka | 2.4714 | 3.42695 | 8.15334 |
| F630111L10Rik | 2.48808 | 3.03509 | 1.19158 |
| Dusp1 | 2.46585 | 3.67892 | 2.53725 |
| Trf | 2.45823 | 3.11267 | 1.95677 |
| Podd | 2.45798 | 3.39572 | 5.5448 |
| Socs3 | 2.45833 | 3.04242 | 3.22108 |
| Adamts9 | 2.44378 | 3.96151 | 15.3594 |
| Pld3 | 2.44099 | 2.98473 | 2.23635 |
| Cd300ld | 2.43881 | 5.21866 | −1.12773 |
| Ms4a7 | 2.43369 | 2.7474 | 1.01425 |
| Cd80 | 2.4207 | 4.97268 | −3.1212 |
| Rps 12 | 2.41828 | 2.84166 | 2.89344 |
| Ptplad2 | 2.41459 | 3.15027 | 1.04208 |
| OTTMUSG00000003806 | 2.41141 | 2.78601 | −1.1132 |
| Cmpk2 | 2.40988 | 2.30472 | 1.51885 |
| Wdr78 | 2.39407 | 2.41427 | 3.14536 |
| Inp4b | 2.3933 | 3.56508 | 3.73708 |
| Clac2d | 2.37606 | 2.50288 | 2.91928 |
| Cylip | 2.36658 | 2.83377 | 1.54389 |
| BhIhe40 | 2.36473 | 3.48673 | 1.98818 |
| Gbo2 | 2.3844 | 3.4082 | 4.17193 |
| Cybb | 2.38245 | 1.1716 | −7.68031 |
| Tspar7 | 2.35842 | 3.5855 | −1.1462 |
| Amox4 | 2.35547 | 2.62818 | 2.49050 |
| Ctla2b | 2.35316 | 2.40732 | 2.80199 |
| Lat | 2.34741 | 2.52008 | 8.6574 |
| Ccr7 | 2.64585 | 3.83581 | 1.47222 |
| Cdkn2c | 2.32918 | 3.04303 | 1.91509 |
| Cyp11a1 | 2.3288 | 2.53459 | −1.84663 |
| Lrp1 | 2.32827 | 2.94168 | −1.41086 |
| Fcgr2b | 2.32865 | 2.46647 | −3.25898 |
| Zpb1 | 2.32529 | 2.6347 | 2.37828 |
| Cd33 | 2.31992 | 2.4125 | −2.13877 |
| Glp1r | 2.31349 | 7.83457 | 26.2717 |
| 9430008C03Rik | 2.30257 | 2.21707 | 1.532 |
| OTTMUSG00000003608 | 2.30169 | 2.68281 | −1.13555 |
| Sifn2 | 2.28765 | 2.78406 | 2.02887 |
| Lrrc6 | 2.28495 | 2.85889 | −1.21047 |
| Tnfat11 | 2.28489 | 2.8348 | 5.08707 |
| Slc 18a1 | 2.28384 | 2.64883 | 5.29893 |
| Ctla2a | 2.28118 | 3.4083 | 2.9915 |
| Rnf217 | 2.26141 | 2.88138 | −1.61253 |
| Vegfa | 2.25941 | 2.81397 | 1.62698 |
| Ikzf2 | 2.2561 | 2.68489 | 3.14891 |
| C1qb | 2.23831 | 3.05601 | −1.60513 |
| Skap1 | 2.23626 | 2.51664 | 3.07894 |
| Gab2 | 2.23244 | 2.48448 | 2.16734 |
| Sdc1 | 2.22611 | 2.10809 | −1.13058 |
| Nrg1 | 2.22557 | 2.93118 | 1.10937 |
| Igfbsp4 | 2.22126 | 2.40842 | 1.97153 |
| Slrpb1 | 2.20868 | 2.70303 | −1.23064 |
| Ttc39c | 2.19483 | 2.70732 | −1.28485 |
| Phf11 | 2.19255 | 2.23748 | 1.54078 |
| Nr4a3 | 2.1913 | 4.38594 | 1.06497 |
| EG435337 | 2.17985 | 2.41573 | 1.08976 |
| Cd200r1 | 2.171 | 2.60659 | 1.09681 |
| Chdn | 2.16753 | 2.527 | 4.46084 |
| Raver2 | 2.168 | 2.73904 | 1.39596 |
| Arhgap26 | 2.16389 | 2.46685 | 3.29386 |
| Ptra11 | 2.15813 | 2.96505 | −1.91786 |
| Mpp1 | 2.1544 | 2.58734 | 4.8107 |
| Adamts9 | 2.15162 | 3.13647 | 11.6705 |
| Cd80 | 2.14416 | 4.31747 | −1.38559 |
| Sirpb1 | 2.1317 | 2.39163 | −1.22972 |
| IIgb2 | 2.12998 | 2.62116 | 1.9461 |
| Sytl3 | 2.12972 | 3.23218 | 17.3119 |
| Dpyst2 | 2.12362 | 2.19928 | 2.29924 |
| Pad2 | 2.12247 | 2.91839 | 1.89634 |
| Pigz | 2.11978 | 2.34099 | 1.27114 |
| Angptl2 | 2.11703 | 2.37334 | 3.28362 |
| Tmem120b | 2.11678 | 2.84665 | 1.97575 |
| Mmp19 | 2.113 | 2.79529 | −1.62937 |
| Scat1 | 2.10684 | 2.0523 | 2.26021 |
| Hit1a | 2.10412 | 2.08257 | 1.04897 |
| Tir1 | 2.10145 | 2.683 | 1.8812 |
| Rps6ka2 | 2.09612 | 2.23815 | −1.00789 |
| Irf9 | 2.09586 | 2.62258 | 1.88028 |
| Hsd11b1 | 2.09524 | 2.62652 | 4.81936 |
| Gzmc | 2.09142 | 4.35551 | 1.22175 |
| Nelo2 | 2.07912 | 3.01835 | 1.20022 |
| Ppic | 2.07751 | 2.25499 | 2.10069 |
| Mix2 | 2.07659 | 2.07768 | 1.14853 |
| Tns1 | 2.07556 | 2.63221 | 1.86787 |
| Dab2 | 2.06941 | 2.60367 | −3.26226 |
| Grn | 2.06936 | 2.14488 | 1.2533 |
| II12rb2 | 2.06759 | 2.91462 | 1.20628 |
| Pmd | 2.66701 | 2.18501 | 2.59987 |
| II1b | 2.06608 | 3.34846 | 1.03578 |
| Maoa | 2.06461 | 2.72903 | 1.34207 |
| 1190002H23Rik | 2.05259 | 2.41921 | 2.8719 |
| Sc8 | 2.04893 | 2.17914 | 2.43976 |
| Pgm2 | 2.04613 | 2.36044 | 2.18478 |
| 5830443L24Rik | 2.04654 | 2.25619 | 4.31877 |
| Casp4 | 2.04181 | 2.18189 | 1.81916 |
| Tgfb2 | 2.03685 | 3.12251 | 3.60565 |
| Itgb3 | 2.03575 | 2.39128 | 4.97243 |
| Alp11a | 2.03244 | 2.37311 | −2.22166 |
| Mgst1 | 2.02468 | 2.48631 | −1.59273 |
| Ulm | 2.024 | 2.56757 | 4.71905 |
| Socs1 | 2.02063 | 2.44114 | 2.86879 |
| Fam102a | 2.0168 | 2.58775 | 2.8841 |
| Adamts17 | 2.01348 | 2.5051 | 1.9104 |
| Slc8a9 | 2.00397 | 2.38152 | 4.25096 |
| Slmp2 | 2.00114 | 2.61137 | 1.75852 |
| Eltd1 | −22.9754 | −16.549 | −27.7951 |
| Gam | −15.8598 | −10.7717 | −18.5538 |
| Car2 | −15.8898 | −10.2728 | −5.41411 |
| II1r1 | −13.8649 | −8.83852 | −20.5475 |
| Ngp | −12.8117 | −13.7997 | −14.044 |
| Scin | −10.0228 | −5.21038 | 1.11904 |
| 8039619P08Rik | −9.82608 | −9.73486 | −5.19994 |
| Angpt1 | −9.25465 | −7.59781 | 1.06172 |
| Hava2 | −8.77255 | −4.81728 | −4.07701 |
| Spint1 | −6.12629 | −7.07769 | −8.73585 |
| Kmo | −7.93663 | −8.59275 | −11.8556 |
| Ccnd1 | −7.25722 | −5.93082 | −8.62119 |
| Ctr9 | −8.99367 | −5.43232 | −8.98698 |
| Mn1 | −6.84299 | −5.85423 | −8.00683 |
| Nrp1 | −6.54268 | −6.87954 | 1.02283 |
| Ela2 | −6.56389 | −5.10979 | −18.2404 |
| Gca | −6.59228 | −4.68177 | −8.97908 |
| P2rx3 | −6.44161 | −5.80453 | −7.99622 |
| Slc7a3 | −6.41689 | −4.68898 | −3.62658 |
| B3gril5 | −6.37376 | −5.20535 | −3.29699 |
| Ly86 | −6.25905 | −3.76832 | −14.6565 |
| Gcat2 | −6.17738 | −8.12654 | −7.38081 |
| Mctp2 | −5.99947 | −5.83887 | −1.40131 |
| Ica1 | −5.97299 | −4.67768 | −12.3424 |
| Cd79b | −5.8064 | −4.72267 | −1.81491 |
| Gm10759 | −5.7678 | −5.50624 | −7.12133 |
| Tmem119 | −5.5754 | −5.23618 | −8.18145 |
| Mcpt8 | −5.33034 | −4.58927 | −1.38813 |
| Cd96 | −5.2597 | −3.51257 | −9.94218 |
| Dusp6 | −5.25522 | −5.09607 | −2.04294 |
| Gatm | −5.24027 | −3.92614 | −18.1972 |
| Chad | −5.23925 | −5.30178 | −2.5267 |
| Slco3a1 | −5.1331 | −4.46489 | −1.26163 |
| Vkllr | −4.92129 | −5.04334 | −5.55231 |
| Slau2 | −4.88021 | −3.89139 | −4.08542 |
| Gapt | −4.6828 | −4.18121 | −21.1742 |
| Ociad2 | −4.59689 | −3.87991 | −4.53921 |
| Lmo2 | −4.59667 | −4.0181 | −8.67691 |
| Irf8 | −4.56289 | −4.37611 | −6.5712 |
| Itih5 | −4.4706 | −3.2061 | 1.53524 |
| Lax1 | −4.41638 | −4.38896 | 1.01314 |
| Gcnt2 | −4.24673 | −3.86277 | −8.19734 |
| #18rap | −4.23046 | −3.31314 | −2.11952 |
| Cfse | −4.15264 | −2.68912 | 2.05945 |
| Etv5 | −4.13064 | −2.9047 | −1.53263 |
| Bex6 | −4.11078 | −2.20867 | −4.64863 |
| Tnfraf13c | −4.08414 | −3.72487 | −4.12606 |
| As3rnt | −4.00969 | −4.10349 | −4.18491 |
| Rgs1 | −3.9685 | −2.07056 | −1.84249 |
| Cdh17 | −3.94059 | −4.27798 | −4.10438 |
| Gyb3 | −3.85075 | −3.69143 | −3.36635 |
| Mtss1 | −3.84501 | −2.60776 | −1.80942 |
| Dhfs3 | −3.84111 | −3.8264 | −1.80484 |
| Dmxf2 | −3.80244 | −3.19718 | −5.15479 |
| Ctnad2 | −3.78921 | −3.11959 | −4.7023 |
| Clnk | −3.77363 | −2.92093 | −4.16011 |
| Enam | −3.75929 | −4.48151 | −4.34622 |
| Abobfb | −3.74209 | −3.5026 | −6.68358 |
| Gimap6 | −3.73656 | −2.82114 | 2.08393 |
| Ccdc135 | −3.69517 | −3.30903 | 1.49684 |
| Etv6 | −3.68519 | −3.35519 | −1.65464 |
| Mef2e | −3.53747 | −3.69193 | −16.2543 |
| Rnase6 | −3.6311 | −2.74826 | −3.38474 |
| Endod1 | −3.59897 | −3.17572 | −1.78752 |
| Eng | −3.57927 | −3.14449 | −2.76283 |
| Cd28 | −3.58383 | −2.31162 | −1.36665 |
| Rsep6 | −3.56251 | −3.18968 | −3.47988 |
| Aidh#2 | −3.52063 | −2.98543 | −6.16953 |
| Arap2 | −3.49241 | −2.04032 | −1.0922 |
| Cth | −3.47913 | −3.87157 | −5.84442 |
| Gm14005 | −3.45878 | −2.90911 | −4.9562 |
| Tbxas1 | −3.45636 | −3.64581 | −3.59535 |
| Pigs1 | −3.45524 | −2.81284 | −3.3679 |
| Npgda | −3.4465 | −2.7336 | −2.45812 |
| Pinf141 | −3.90482 | −2.50775 | −3.86436 |
| #trt2 | −3.35409 | −3.24307 | −1.79612 |
| Freq | −3.30662 | −2.21751 | −4.70611 |
| St3gal5 | −3.29532 | −2.42743 | −2.33915 |
| Cd300c | −3.28524 | −2.88462 | −3.80494 |
| Nos1ap | −3.25637 | −3.19264 | −5.29064 |
| Car1 | −3.24057 | −3.46657 | −3.79614 |
| Rnf141 | −3.22214 | −3.08287 | −4.44614 |
| Adamis3 | −3.19572 | −2.33096 | 2.39393 |
| Aicam | −3.18341 | −2.30949 | −3.53906 |
| Trem3 | −3.18303 | −2.65477 | −9.50106 |
| Tipa | −3.15576 | −2.62588 | −2.82279 |
| Khdrba3 | −3.15538 | −2.86313 | −3.79078 |
| Eomes | −3.15433 | −2.84249 | −3.99267 |
| Wwo2 | −3.14056 | −2.51645 | −2.85619 |
| Elov17 | −3.13961 | −3.90645 | −3.44777 |
| Tnfrsf10b | −3.13611 | −2.55724 | −3.1209 |
| Rhobtb3 | −3.13066 | −2.5144 | −2.66067 |
| Map4k5 | −3.12595 | −2.33849 | −7.26474 |
| AI747699 | −3.11628 | −2.30268 | −4.16657 |
| Hpse | −3.09238 | −3.05695 | −2.65227 |
| Ppp2r5c | −3.07913 | −2.66575 | −2.94178 |
| Fmnt2 | −3.06013 | −2.5278 | 2.64353 |
| 5lc25at2 | −3.05852 | −2.17354 | −1.79452 |
| Ttps | −3.05865 | −3.10218 | −2.4944 |
| Hist2h3c2 | −3.05006 | −2.89553 | −1.79297 |
| Cd180 | −3.04738 | −2.21009 | −9.70304 |
| Gcnt1 | −3.04326 | −2.36856 | −3.81737 |
| Pycr1 | −3.01456 | −2.62363 | −3.76368 |
| Lat2 | −3.01065 | −2.71517 | −1.90048 |
| Ncf1 | −3.00679 | −2.39271 | −7.62038 |
| Zfp111 | −3.00597 | −2.5174 | −4.11985 |
| EG668725 | −2.99877 | −2.203 | −3.8484 |
| Bcl11a | −2.99062 | −2.57149 | −5.3496 |
| Sic14at | −2.98672 | −2.23222 | 2.38665 |
| Sic7a11 | −2.97918 | −2.85393 | −4.09816 |
| Ptgar3 | −2.93257 | −3.28815 | −2.56727 |
| Hif | −2.91454 | −3.06227 | −3.58412 |
| Fmnl3 | −2.90735 | −2.17705 | 1.09454 |
| Adamts3 | −2.89844 | −2.14201 | 1.76691 |
| Ptp4a3 | −2.89151 | −2.75511 | −1.8058 |
| Cd5 | −2.57597 | −2.68734 | −1.39656 |
| Frmd4b | −2.83584 | −2.2674 | −2.61587 |
| Mpagf | −2.83262 | −2.47308 | −27.948 |
| Itga1 | −2.82851 | −2.3219 | −4.84402 |
| Rhf122 | −2.80054 | −2.28414 | −1.73369 |
| Cybasc3 | −2.79134 | −2.46935 | −2.30818 |
| Ly6d | −2.78845 | −2.30935 | 4.8948 |
| Sic9a7 | −2.77314 | −2.05624 | −2.09647 |
| Tifab | −2.77547 | −2.22333 | −8.31701 |
| Gli1b | −2.75957 | −2.35573 | −3.12904 |
| Pikce | −2.75687 | −2.83787 | −1.87411 |
| Pbida3 | −2.74183 | −2.20357 | −2.68038 |
| Step1 | −2.74157 | −2.46328 | −1.4477 |
| Adc | −2.72588 | −2.25207 | −2.6314 |
| Ppp2r5c | −2.71959 | −2.59867 | −2.62668 |
| Myl4 | −2.71132 | −2.74636 | 1.40395 |
| | −2.70973 | −2.54579 | −2.16378 |
| Ctdn12 | −2.70709 | −2.51861 | −2.23501 |
| #21r | −2.69951 | −2.30324 | −2.13947 |
| Chn2 | −2.68912 | −2.63875 | −1.27931 |
| Atp2b4 | −2.68845 | −2.03103 | −1.93488 |
| Gllf3a | −2.68504 | −2.05468 | −2.52224 |
| AY512938 | −2.67329 | −2.00539 | −3.8029 |
| Tat1 | −2.65904 | −2.0499 | −3.74064 |
| Sic2a3 | −2.66753 | −2.22851 | −1.78188 |
| Mpp8 | −2.65092 | −2.42796 | −3.20545 |
| II12a | −2.65931 | −2.35042 | −6.06814 |
| Cd244 | −2.64561 | −2.08854 | −1.63998 |
| Sic1a4 | −2.62993 | −2.25252 | −2.89647 |
| Tpca1 | −2.82521 | −2.04259 | −1.00642 |
| 4632434l11Rik | −2.59649 | −2.28398 | −3.99785 |
| Sdr39u1 | −2.58194 | −2.00131 | −1.59182 |
| Nuch2 | −2.57662 | −2.28293 | −3.04431 |
| Hbb-b1 | −2.5802 | −2.11759 | 1.5637 |
| Spns2 | −2.55629 | −2.4746 | −1.28595 |
| Rasgrp2 | −2.54754 | −2.19521 | 1.06949 |
| Mansc1 | −2.54631 | −2.37535 | −2.33869 |
| Atap1I1 | −2.52469 | −2.15397 | −1.56508 |
| Syp1 | −2.51859 | −2.3583 | −1.31649 |
| Eat2 | −2.49569 | −2.92504 | −4.44764 |
| Xlr | −2.49399 | −2.07904 | 1.36081 |
| Pla2g4a | −2.46737 | −2.33143 | −3.0182 |
| 8330442E10Rik | −2.45528 | −2.20305 | −1.83388 |
| Tax3 | −2.4548 | −2.18718 | −1.32199 |
| Tmam74 | −2.45489 | −2.23676 | −2.35782 |
| Abcg2 | −2.44894 | −2.16995 | −2.1362 |
| Adamts3 | −2.39972 | −2.17619 | 1.88526 |
| Mtap7d3 | −2.39671 | −2.38899 | −3.38961 |
| Dtx4 | −2.39635 | −2.09732 | −2.66278 |
| Tbxa2r | −2.38262 | −2.61988 | 1.65776 |
| Rnf144b | −2.3769 | −2.19419 | −1.89628 |
| Lphn2 | −2.36277 | −2.13425 | −2.07239 |
| Gm14636 | −2.35898 | −2.44458 | −2.33529 |
| Agpat2 | −2.34969 | −2.03637 | −1.42651 |
| Z2104l1K11Rik | −2.33616 | −2.10374 | −2.56884 |
| Egft7 | −2.32044 | −2.34124 | −2.56469 |
| Cox6a2 | −2.31998 | −2.60147 | −7.61449 |
| Gstm5 | −2.30962 | −2.45877 | −2.75549 |
| Chac1 | −2.36493 | −2.24616 | −3.58965 |
| Bmyc | −2.30408 | −2.03438 | −3.83447 |
| Sic5a9 | −2.29084 | −2.29089 | −1.59005 |
| Emp1 | −2.27769 | −2.24488 | −2.18041 |
| Dmxf2 | −2.26424 | −2.14548 | −2.55869 |
| Msrb3 | −2.2464 | −2.24847 | −2.72617 |
| Dik1 | −2.23043 | −2.27512 | −1.6005 |
| Jam2 | −2.22996 | −2.12628 | −2.27274 |
| Aqp9 | −2.22371 | −2.00945 | −2.6499 |
| Fam59a | −2.20936 | −2.00631 | −1.70136 |
| Fecr | −2.20797 | −2.02732 | −2.1963 |
| Igj | −2.19759 | −2.35463 | −1.22972 |
| Tspan6 | −2.19192 | −2.1279 | −2.56323 |
| Stc2 | −2.18712 | −2.14602 | −1.81667 |
| Gm4989 | −2.18422 | −2.26545 | −1.34541 |
| Asns | −2.17183 | −2.18641 | −3.47034 |
| Coq3 | −2.13843 | −2.06335 | −2.365 |
| Ctsg | −2.12152 | −2.4833 | −3.19363 |
| Ifrd1 | −2.10407 | −2.04786 | −2.13253 |
| Cops4 | −2.08808 | −2.02481 | −2.03318 |
| Rhobtb1 | −2.05878 | −2.00695 | −1.9559 |
| Mc5r | −2.52504 | −2.13056 | −1.71937 |
| Rab39 | −2.00869 | −2.32875 | −1.67399 |
| Mina | −2.09379 | −2.07573 | −2.65565 |

### Example 2

### Inducible TCF-1 Expression

Transcription factors that function as master regulators are essential in the establishment of genetic networks that commit progenitors to a particular lineage. These transcription factors are defined by their key functions in that particular cell lineage, but whether these regulators are required to maintain the lineage program that they establish is often unknown. As described elsewhere herein, TCF-1 is critical for the initiation of normal T cell development and sufficient to induce T-lineage specific gene expression. Indeed, TCF-1 is expressed in all mature T-lineages.

### Validation of an Inducible TCF-1-ER Construct

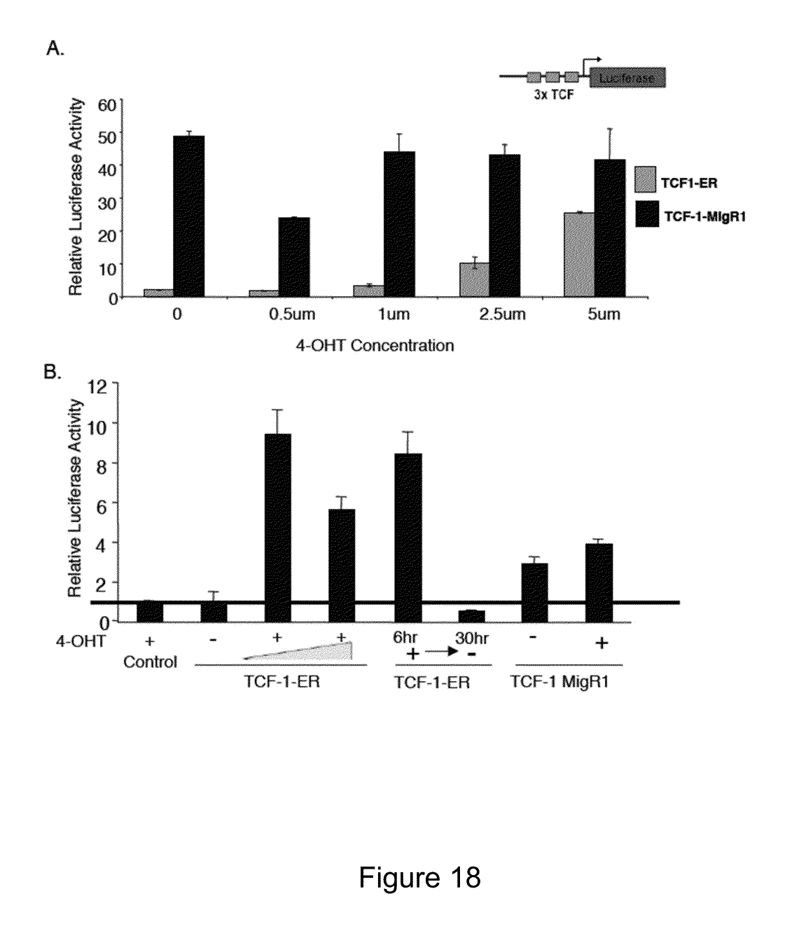

An inducible TCF-1 system is described herein. Human TCF-1 was fused to the estrogen receptor (ER) at the N-terminus and cloned into the MSCV-GFP (MiGR1) vector. In this system, TCF-1 is constitutively expressed but localized in the cytoplasm with ER, perhaps due to association with heat shock proteins. In the presence of tamoxifen (ER agonist), TCF-1-ER will translocate to the nucleus where it will be constitutively maintained, as long as tamoxifen is present. To validate this system, the ability of TCF-1-ER-GFP to activate the TCF-1 reporter, TOPFLASH, in 293T fibroblasts, was assessed. In the absence of 4-hydroxytamoxifen (4-OHT), the active metabolite of tamoxifen, TCF-1-ER fails to activate luciferase transcription. However, in the presence of 4-OHT, TCF-1-ER activates in a dose dependent manner. As a control, TCF-1 MiGR1 was transfected and it was observed that at Sum concentration, the TCF-1-ER construct activated close to TCF-1 MIGR1 levels (FIG. 18A).

Next, the ability to remove TCF-1 when 4-OHT was washed out of the cultures in vitro was assessed. A similar TCF-1 reporter assay was utilized in which TCF-1 binding sites were integrated into the genome of 293T. The ability of TCF-1 to activate this reporter was assayed, along with the kinetics upon removal of 4-OHT from the cultures. 4-OHT was removed at thirty hours and six hours prior to harvest by washing and replacing with fresh media. Similar to previous results, TCF-1-ER failed to activate luciferase activity in the absence of 4-OHT and addition of 4-OHT activated the reporter ten-fold. In addition, removal of 4-OHT thirty hours before harvest was sufficient to decrease luciferase activity back to background, whereas at 6 hours, this was unchanged (FIG. 18B). These data confirm that addition of 4-OHT is able to induce TCF-1 activity and that removal of 4-OHT from culture medium is sufficient to reverse activity within one day.

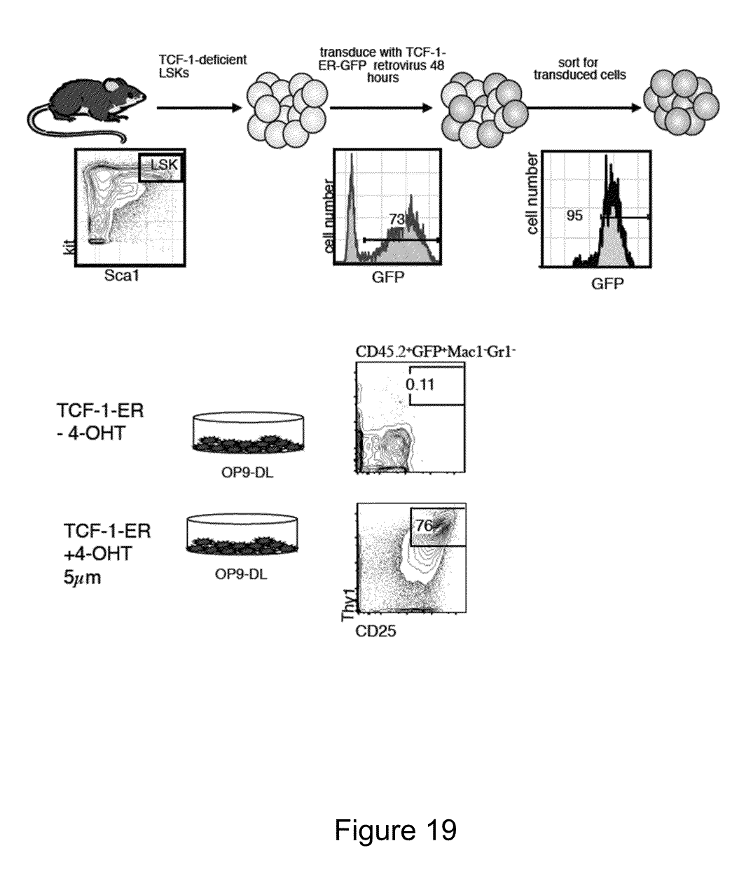

TCF-1-deficient progenitors exhibit a severe phenotype in vitro with an absolute defect in the ability to generate T cell progenitors on OP9-DL1 stroma. To address whether the TCF-1-ER inducible system would rescue the T-lineage defect, TCF-1−/− LSK progenitors were isolated and transduced with TCF-1-ER. Transduced progenitors were seeded onto OP9-DL1 stoma in the presence or absence of Sum 4-OHT. In the absence of 4-OHT, TCF-1-ER-expressing progenitors failed to give rise to any Thy1+CD25+ T-lineage cells. However, addition of 4-OHT rescued T cell development, demonstrated by the presence of Thy1+CD25+ T-lineage cells (FIG. 19). The failure to observe any T cell development in the absence of 4-OHT in the medium suggests that this construct is not leaky by this readout. The ability of TCF-1-ER to restore T cell development in vitro indicates that this construct can be used to generate T cell progenitors and assay the consequence of removal of TCF-1.

To determine whether TCF-1-ER would drive T cell development in the absence of Notch1 ligands, TCF-1-deficient TCF-1-ER-expressing progenitors were plated on OP9 stroma in the presence of Sum 4-OHT. Thy1+CD25+ T-lineage cells were not observed in the presence of Sum 4-OHT, although TCF-1 was sufficient to inhibit B cell development. These data are consistent with observations that TCF-1 in the MiGR1 construct drives TCF-1 expression at levels approximately five-fold lower than the TCF-1 VEX construct. Here, TCF-1-GFP was also able to block B cell development, but not induce Thy1+CD25+ T-lineage development. These data demonstrate the importance of TCF-1 levels and further highlight the synergism between Notch1 and TCF-1, as lower levels of TCF-1 are able to restore T cell development when Notch signals are present.

### Loss of TCF-1 Diverts T Cell Progenitors to the Myeloid Fate Despite Active Notch Signals

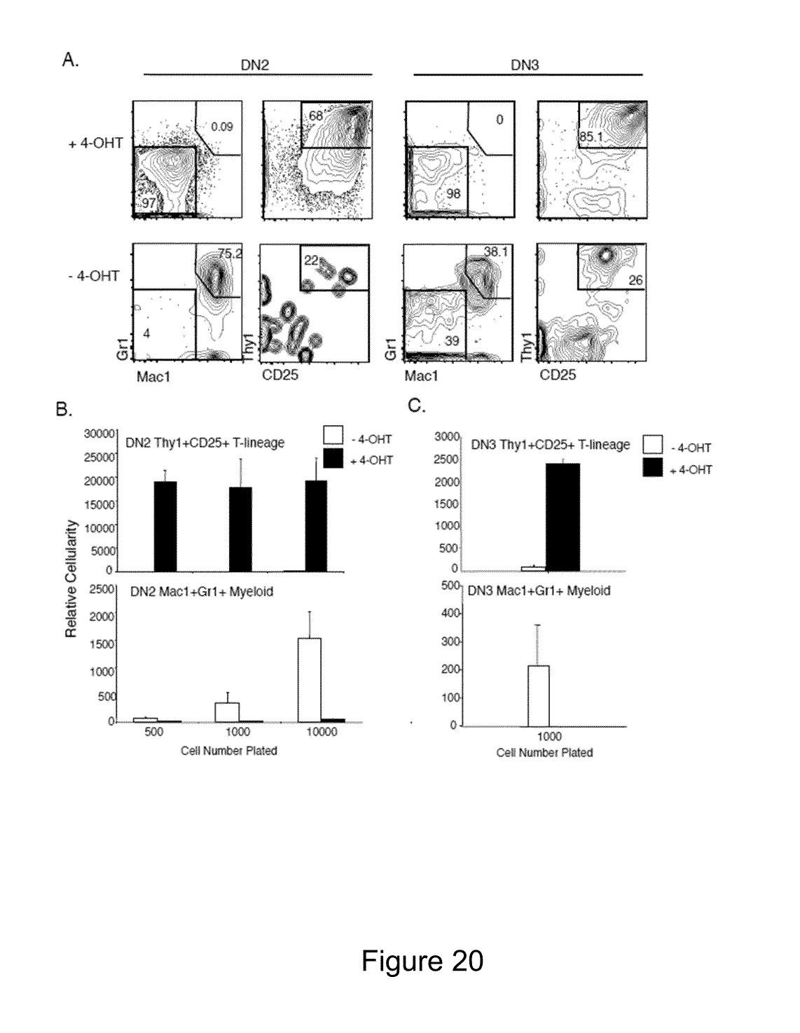

To determine the functional outcome of loss of TCF-1 expression in a T cell progenitor, TCF-1-ER-expressing T cell progenitors were generated. To do this, TCF-1-ER was ectopically expressed in TCF-1-deficient LSKs and the TCF-1-ER-expressing progenitors were cultured on OP9-DL1 stroma in the presence of 4-OHT for two weeks. As a control, progenitors were plated without 4-OHT and it was confirmed that no T cells were generated. Then DN2 (CD44+CD25+) and DN3 cells (CD44−CD25+) were isolated by cell sorting and replated back onto OP9-DL1 stroma in the presence of IL-7 and Flt3L. Cultures were analyzed for T cell development one week later. T cell development was almost entirely abolished in wells containing DN2 and DN3 T cell progenitors that had been cultured in the absence of 4-OHT (FIG. 20). Instead, the presence of Mac1+Gr1+ myeloid cells was observed. Multiple cell doses were tested, and in each scenario, reversing TCF-1 expression resulted in the loss of T cell progenitors and diversion to the myeloid lineage. The overall cellularity of these cultures was much lower than the cultures in which 4-OHT was added. Here, all cells developing were T-lineage cells. The presence of Thy1+CD25+ T-lineage cells that continued to proliferate and expand in the wells containing 4-OHT is consistent with the explanation that the T-cell progenitors were being exposed to active Notch1 signals.

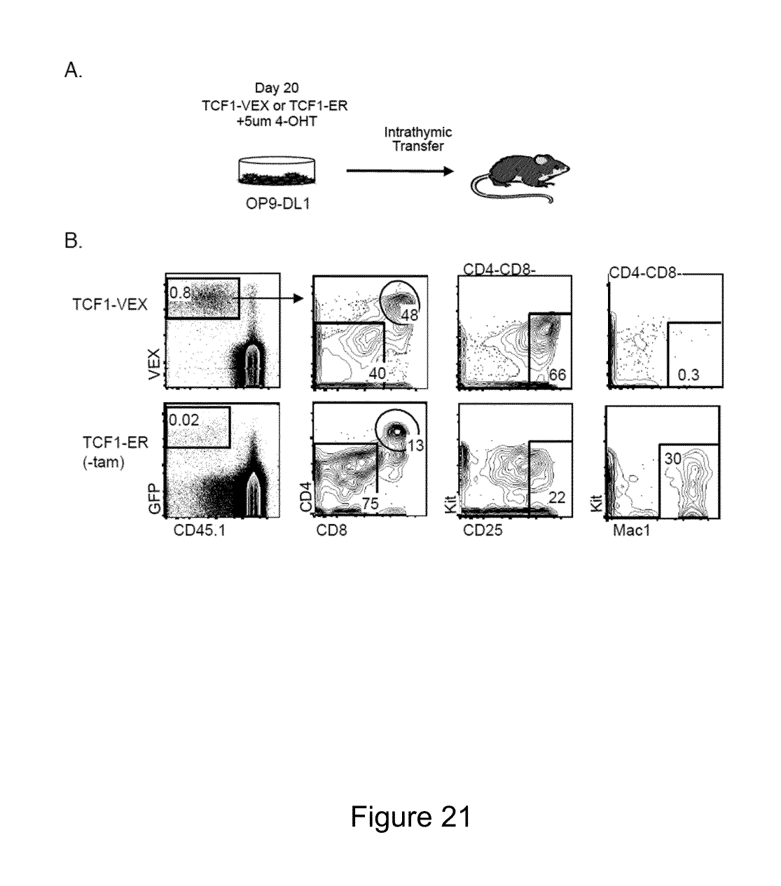

TCF-1-ER-expressing Thy1+CD25+ T-lineage cells and TCF-1-VEX expressing Thy1+CD25+ T-lineage cells were generated. The latter population constitutively express TCF-1, and allowed the assessment of functional consequences when these T-lineage cells are injected intrathymically in the absence of tamoxifen. The experiment was performed in this manner because tamoxifen treatment in vivo has not been able to reliably restore TCF-1 expression in the TCF-1-ER expressing progenitors, because the concentrations of tamoxifen are not high enough. T cell progenitors from TCF-1-ER and TCF-1 VEX OP9-DL1 cultures were injected into sublethally irradiated congenic mice. In other experiments, TCF-1-VEX expressing T cells from in vitro OP9-DL1 cultures were injected and it was demonstrated that this population continues T cell development similar to wild-type T cell progenitors. T cell reconstitution was analyzed eleven days later. TCF-1-VEX expressing donor cells were found to continue T cell development, generating DP thymocytes and DN3 thymocytes at the timepoints examined (FIG. 21). Consistent with the in vitro experiments, TCF-1-ER donor cells generated few DP thymocytes and gave rise to a population of Mac1+(CD4−CD8−CD25−) cells within the thymus.

### Compensatory Upregulation of LEF-1 May Provide a Partial Rescue In Vitro in the Absence of TCF-1

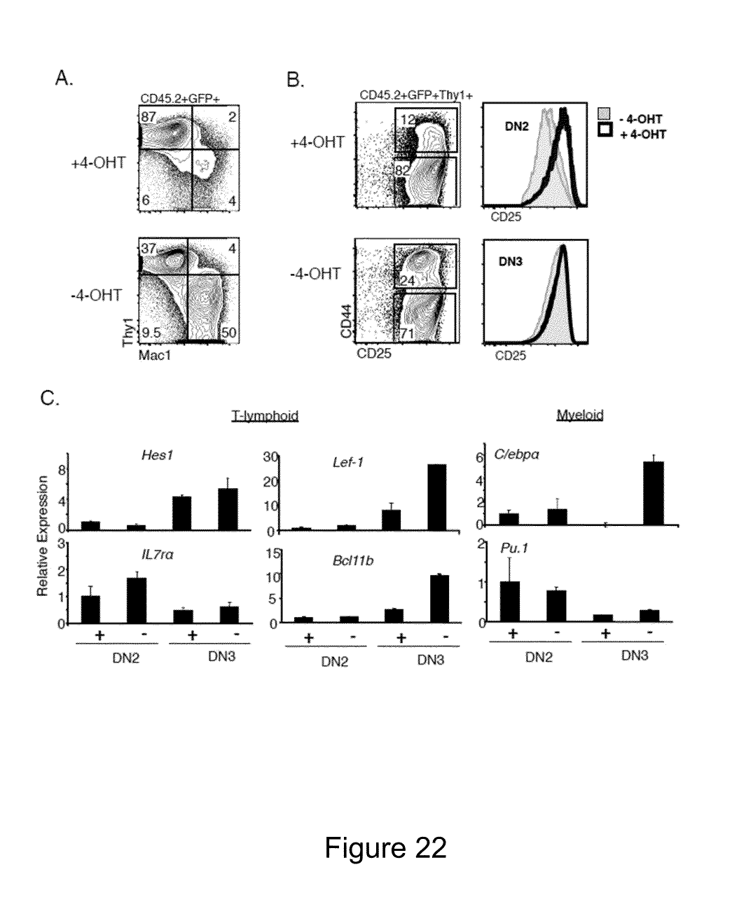

The results described elsewhere herein have revealed a dramatic requirement for TCF-1 in early T-lineage progenitors. However, some of these experiments were performed using limiting cell numbers after cell-sorting DN2 or DN3 progenitors and replating them back onto OP9-DL1 stroma. The consequence of removal of 4-OHT from bulk cultures has also been addressed, in which approximately 1-3 million cells are developing. In these experiments described here, Thy1+CD25+ T-lineage cells were generated by transducing TCF-1-deficient LSKs with TCF-1-ER and then the transduced cells were transferred onto OP9-DL1 stroma in the presence of 5 μm 4-OHT. After two weeks of induction, total bulk cultures were passaged and placed back onto OP9-DL1 stroma in the presence or absence of 4-OHT. To assess where T cells were still developing, TCF-1 was withdrawn from total bulk cultures. In the experiments described here, all cells that were still developing in culture were switched from medium with 4-OHT to medium without 4-OHT, including all non-DN2/DN3 lineage cells, and the cell number was over one hundred fold greater. After one week, the cultures were assessed for T and myeloid cell development. DN2 and DN3 T cell progenitors were still developing in the wells without 4-OHT in the culture medium (FIG. 22). In addition, an abundant population of Mac1+ cells was observed in culture that was absent in wells that contained 4-OHT, consistent with the earlier experiments. A difference in CD25 expression was noted between DN2 progenitors with lower levels of cell surface CD25 expression in the cultures in which 4-OHT was withdrawn. Indeed, the difference in CD25 expression was observed within twenty-four hours of removal of 4-OHT. DN2 and DN3 progenitors were also isolated from these cultures twelve days after the first passage to assess gene expression. Interestingly, it was observed that the DN2 and DN3 cells from cultures that did not contain 4-OHT in the culture medium expressed higher levels of LEF-1 compared to the cells developing in the presence of 4-OHT. LEF-1 was also higher in DN3 thymocytes compared to DN2 thymocytes. This is consistent with the explanation that during thymocyte development, LEF-1 is upregulated at the DN2-DN3 transition. However, IL7rα and the Notch1 target, Hes1, were similar. Interestingly, preliminary data also suggest that the DN3 thymocytes cultured without 4-OHT express higher levels of the myeloid specific transcription factor, C/ebpα compared to DN3 cells in which TCF-1 expression was maintained. This is consistent with the explanation of negative regulatory input from TCF-1 to C/epbα.

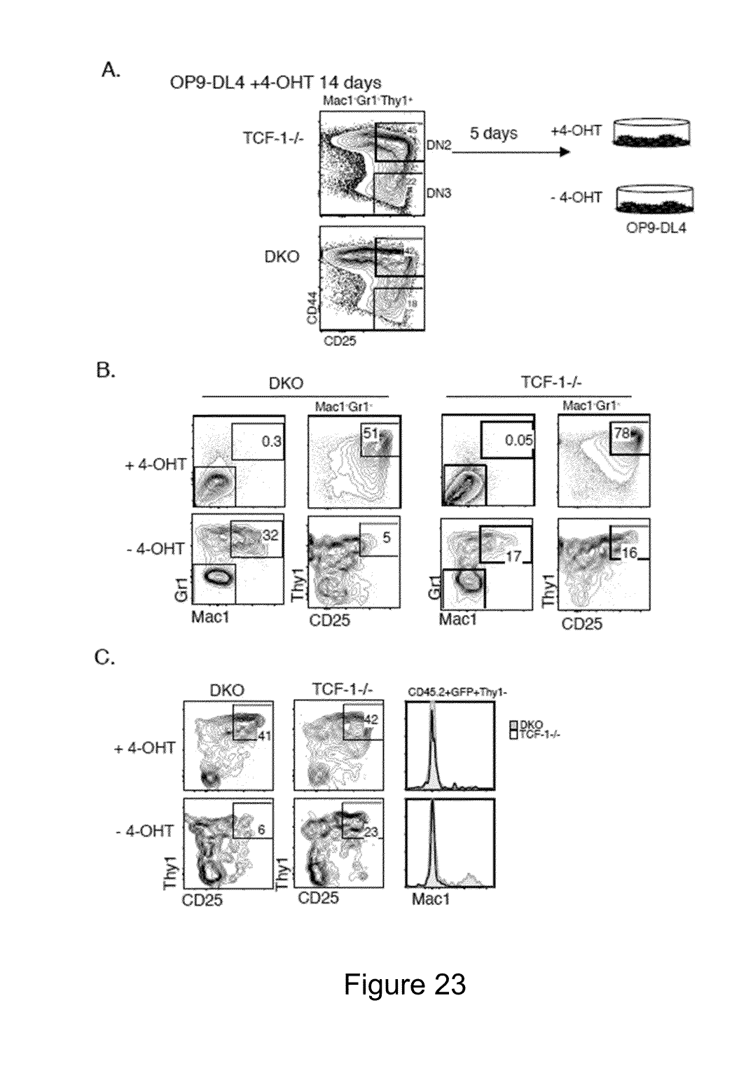

To assess whether LEF-1 compensates in the absence of TCF-1, experiments were performed comparing TCF-1-deficient mice and TCF-1-deficient LEF-1F/FVavCre mice in which LEF-1 is conditionally deleted at the onset of hematopoiesis. LEF-1-deficient mice are embryonic lethal and conditional deletion allows assessment of the hematopoietic effects of LEF-1 deficiency since these mice are viable and develop normally. Both genotypes were transduced with TCF-1-ER and progenitors were cultured on OP9-DL1 stroma in the presence of 4-OHT and supporting cytokines for two weeks. DN2 and DN3 progenitors were isolated by cell sorting and seeded back onto OP9-DL1 in the presence or absence of 4-OHT (FIG. 23A). Consistently, loss of TCF-1 in DN2 progenitors resulted in a diversion to the myeloid fate (FIG. 23B). The phenotype was striking with just loss of TCF-1 in the DN2 progenitors, although LEF-1 deficiency resulted in the appearance of more myeloid cells and a lower frequency of remaining T cells at this timepoint. In the DN3 progenitors, loss of LEF-1 accelerated the appearance of myeloid cells, as only the cells that were deficient in both LEF-1 and TCF-1 had upregulated Mac1 upregulation at this early timepoint (day 5) (FIG. 23C). These results are consistent with the explanation that LEF-1 is able to compensate in the absence of TCF-1. The data described herein demonstrate that T cells that continue to develop express higher levels of LEF-1.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A genetically modified T cell progenitor cell (TCPC) comprising a vector comprising a nucleic acid encoding at least one selected from the group consisting of T Cell Factor (TCF)-1, TCF-3, TCF-4 and TCF-10.

2. The genetically modified TCPC of claim 1, wherein the nucleic acid encodes TCF-1 and wherein the nucleic acid encoding TCF-1 comprises the nucleic acid sequence of SEQ ID NO:37, or a modification thereof.

3. The genetically modified TCPC of claim 1, wherein the genetically modified TCPC is at least one cell selected from the group consisting of an embryonic stem cell (ESC), an induced pluripotent stem cell (iPSC), a hematopoietic stem cell (HSC), a hematopoietic progenitor cell (HPC), a common lymphoid progenitor cell (CLP), an early lymphoid progenitor cell (ELP), an early thymic progenitor cell (ETP), a lymphoid-primed multipotent progenitor cell (LMPP) and a lineage marker-negative cell (LSK).

4. The genetically modified TCPC of claim 1, wherein the genetically modified TCPC is stably transfected.

5. The genetically modified TCPC of claim 4, wherein the vector is at least one vector selected from the group consisting of a retroviral vector and a lentiviral vector.

6. The genetically modified TCPC of claim 1, wherein the genetically modified TCPC is transiently transfected.

7. The genetically modified TCPC of claim 6, wherein the vector is at least one vector selected from the group consisting of a mRNA and a plasmid.

8. A progeny cell derived from the TCPC of claim 1.

9. A T cell derived from the TCPC of claim 1.

10. The T cell of claim 9, wherein the T cell expresses at least one cell surface marker selected from the group consisting of CD2, CD3, CD25, CD4 and CD8.

11. A method of deriving a T cell from a TCPC comprising the steps of: contacting a TCPC with a vector comprising a nucleic acid encoding a polypeptide selected from the group consisting of T Cell Factor (TCF)-1, TCF-3, TCF-4 and TCF-10, allowing the vector comprising the nucleic acid encoding the polypeptide to enter the nucleus of the TCPC, allowing the nucleic acid encoding the polypeptide to be expressed in the TCPC, culturing the TCPC, isolating a progeny cell from the culture, detecting a T cell specific cell surface marker on the progeny cell, thereby deriving a T cell from a TCPC.

12. The method of claim 11, wherein the nucleic acid encoding the polypeptide encodes TCF-1 and wherein TCF-1 comprises the nucleic acid sequence of SEQ ID NO:37, or a modification thereof.

13. The method of claim 11, wherein the TCPC is at least one cell selected from the group consisting of an embryonic stem cell (ESC), an induced pluripotent stem cell (iPSC), a hematopoietic stem cell (HSC), a hematopoietic progenitor cell (HPC), a common lymphoid progenitor cell (CLP), an early lymphoid progenitor cell (ELP), an early thymic progenitor cell (ETP), a lymphoid-primed multipotent progenitor cell (LMPP) and a lineage marker-negative cell (LSK).

14. The method of claim 11, wherein the TCPC is stably transfected with the nucleic acid encoding the polypeptide.

15. The method of claim 14, wherein the vector is at least one vector selected from the group consisting of a retroviral vector and a lentiviral vector.

16. The method of claim 11, wherein the TCPC is transiently transfected with the nucleic acid encoding the polypeptide.

17. The method of claim 16, wherein the vector is at least one vector selected from the group consisting of a mRNA and a plasmid.

18. A progeny cell derived from the method of claim 11.

19. A T cell derived from the method of claim 11.

20. The T cell of claim 19, wherein the T cell expresses at least one cell surface marker selected from the group consisting of CD2, CD3, CD25, CD4 and CD8.

21. A method of treating a subject with a disease or disorder, comprising the step of administering to the subject at least one T cell derived from a genetically modified TCPC, wherein the genetically modified TCPC comprises a nucleic acid encoding at least one polypeptide selected from the group consisting of T Cell Factor (TCF)-1, TCF-3, TCF-4 and TCF-10.

22. The method of claim 21, wherein the nucleic acid encoding the polypeptide encodes TCF-1 and wherein TCF-1 comprises the nucleic acid sequence of SEQ ID NO:37, or a modification thereof.

23. The method of claim 21, wherein the genetically modified TCPC is at least one cell selected from the group consisting of an embryonic stem cell (ESC), an induced pluripotent stem cell (iPSC), a hematopoietic stem cell (HSC), a hematopoietic progenitor cell (HPC), a common lymphoid progenitor cell (CLP), an early lymphoid progenitor cell (ELP), an early thymic progenitor cell (ETP), a lymphoid-primed multipotent progenitor cell (LMPP) and a lineage marker-negative cell (LSK).

24. The method of claim 21, wherein the genetically modified TCPC is stably transfected.

25. The method of claim 24, wherein the vector is at least one vector selected from the group consisting of a retroviral vector and a lentiviral vector.

26. The method of claim 21, wherein the genetically modified TCPC is transiently transfected.

27. The method of claim 26, wherein the vector is at least one vector selected from the group consisting of a mRNA and a plasmid.

28. The method of claim 21, wherein the T cell expresses at least one cell surface marker selected from the group consisting of CD2, CD3, CD25, CD4 and CD8.

29. The method of claim 21, wherein the disease or disorder comprises T cell deficiency.

30. The method of claim 29, wherein the disease of disorder comprising T cell deficiency is at least one selected from the group consisting of T cell deficiency following bone marrow ablation, T cell deficiency following bone marrow transplant, T cell deficiency following chemotherapy, and T cell deficiency following corticosteroid therapy.
